SEQUENCE LISTING (3 sequences)

SEQ ID NO: 1 - DNA, 22 bp, Homo sapiens
caacatgaatgccattttccaa

SEQ ID NO: 2 - DNA, 21 bp, Homo sapiens
ataatcccctgaacccaagga

SEQ ID NO: 3 - DNA, 25 bp, Homo sapiens
taaagccatgccctctgcaggaaca

(19) United States
(12) Patent Application Publication
(10) Pub. No.: US 2011/0112135 A1
(43) Pub. Date: May 12, 2011

(54) Imidazo [1,2-A] Pyridine Compounds

(76) Inventors: Robert Ray Singhaus, JR., Pottstown, PA (US); Ronald Charles Bernotas, Wayne, PA (US); Jay Edward Wrobel, Lawrenceville, NJ (US); Robert John Steffan, Langhorne, PA (US); Edward Martin Matelan, Royersford, PA (US)

(21) Appl. No.: 12/809,893

(22) PCT Filed: Dec. 19, 2008
(86) PCT No.: PCT/US08/87708

§ 371 Date: Nov. 4, 2010

Related U.S. Application Data
(60) Provisional application No. 61/015,850, filed on Dec. 21, 2007.

(51) Int. Cl. A61K 31/4436; C07D 471/02; A61K 31/44; A61Q 19/00; A61Q 19/08

(52) U.S. Cl. 514/300; 546/121

(57) ABSTRACT

This invention relates generally to imidazo[1,2-a]pyridine-based modulators of Liver X receptors (LXRs) having formula (I) and related methods:

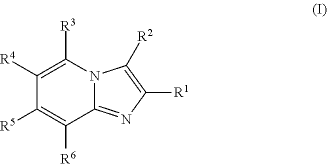

wherein R² is C₆-C₁₀ aryl or heteroaryl including 5-10 atoms, each of which is: (i) substituted with 1 R⁷, and (ii) optionally substituted with from 1-5 R^{e}; and R¹, R³, R⁴, R⁵, R⁶, R⁷, and R^{e} are defined herein.

### CROSS REFERENCE TO RELATED APPLICATIONS

This application is the U.S. National Phase filing under 35 U.S.C. §371 of PCT/US2008/087708 filed Dec. 19, 2008 which claims the benefit of U.S. 61/015,850, filed Dec. 21, 2007, the entire contents of which is incorporated herein by reference.

### TECHNICAL FIELD

This invention relates generally to imidazo[1,2-a]pyridine-based modulators of Liver X receptors (LXRs) and related methods.

### BACKGROUND

Atherosclerosis is among the leading causes of death in developed countries. Some of the independent risk factors associated with atherosclerosis include the presence of relatively high levels of serum LDL cholesterol and relatively low levels of serum HDL cholesterol in affected patients. As such, some anti-atherosclerotic therapy regimens include the administration of agents (e.g., statins) to reduce elevated serum LDL cholesterol levels.

Agents that increase patient HDL cholesterol levels can also be useful in anti-atherosclerotic therapy regimens. HDL cholesterol is believed to play a major role in the transport of cholesterol from peripheral tissues to the liver for metabolism and excretion (this process is sometimes referred to as “reverse cholesterol transport”). ABCA1 is a transporter gene involved in HDL production and reverse cholesterol transport. Upregulation of ABCA1 can therefore result in increased reverse cholesterol transport as well as inhibition of cholesterol absorption in the gut. In addition, HDL is also believed to inhibit the oxidation of LDL cholesterol, reduce the inflammatory response of endothelial cells, inhibit the coagulation pathway, and promote the availability of nitric oxide.

Liver X receptors (LXRs), originally identified in the liver as orphan receptors, are members of the nuclear hormone receptor super family and are believed to be involved in the regulation of cholesterol and lipid metabolism. LXRs are ligand-activated transcription factors and bind to DNA as obligate heterodimers with retinoid X receptors. While LXRα is generally found in tissues such as liver, kidney, adipose tissue, intestine and macrophages, LXRβ displays a ubiquitous tissue distribution pattern. Activation of LXRs by oxysterols (endogenous ligands) in macrophages results in the expression of several genes involved in lipid metabolism and reverse cholesterol transport including the aforementioned ABCA1; ABCG1; and ApoE. See, e.g., Koldamova, et al., J. Biol. Chem. 2003, 278, 13244.

Studies have been conducted in LXRα knock-out (k/o), LXRβ k/o and double k/o mice to determine the physiological role of LXRs in lipid homeostasis and atherosclerosis. The data from these studies suggested that in double k/o mice on normal chow diet, increased cholesterol accumulation was observed in macrophages (foam cells) of the spleen, lung and arterial wall. The increased cholesterol accumulation was believed to be associated with the presence of reduced serum HDL cholesterol and increased LDL cholesterol, even though the total cholesterol levels in the mice were about normal. While LXRα k/o mice did not appear to show significant changes in hepatic gene expression, LXRβ k/o mice showed 58% decrease in hepatic ABCA1 expression and 208% increase in SREBP1c expression suggesting that LXRβ may be involved in the regulation of liver SREBP1c expression.

Data obtained from studies employing two different atherosclerotic mouse models (ApoE k/o and LDLR k/o) suggest that agonists of LXRα or β can be relatively effective in upregulating ABCA1 expression in macrophages. For example, inhibition of atherosclerotic lesions could be observed when ApoE k/o and LDLR k/o mice were treated with LXRα or β agonists for 12 weeks. The tested agonists were observed to have variable effects on serum cholesterol and lipoprotein levels and appeared to cause a relatively significant increase in serum HDL cholesterol and triglyceride levels. These in vivo data were found to be consistent with in vitro data obtained for the same agonists in macrophages.

In addition to the lipid and triglyceride effects described above, it is also believed that activation of LXRs results in the inhibition of inflammation and proinflammatory gene expression. This hypothesis is based on data obtained from studies employing three different models of inflammation (LPS-induced sepsis, acute contact dermatitis of the ear and chronic atherosclerotic inflammation of the artery wall). These data suggest that LXR modulators can mediate both the removal of cholesterol from the macrophages and the inhibition of vascular inflammation.

For a review of LXR biology and LXR modulators, see, e.g., Goodwin, et al., Current Topics in Medicinal Chemistry 2008, 8, 781; and Bennett, et al., Current Medicinal Chemistry 2008, 15, 195.

For studies related to atherosclerosis, see, e.g., Scott, J. N. Engl. J. Med. 2007, 357, 2195; Joseph, et al., PNAS 2002, 99, 7604; Tangirala, et. al., PNAS, 2002, 99, 11896; and Bradley, et al., Journal of Clinical Investigation 2007, 117, 2337-2346.

For studies related to inflammation, see, e.g., Fowler, et al., Journal of Investigative Dermatology 2003, 120, 246; and US 2004/0259948.

For studies related to Alzheimer's disease, see, e.g., Koldamova, et al., J. Biol. Chem. 2005, 280, 4079; Sun, et al., J. Biol. Chem. 2003, 278, 27688; and Riddell, et al., Mol. Cell Neurosci. 2007, 34, 621.

For studies related to diabetes, see, e.g., Kase, et al., Diabetologia 2007, 50, 2171; and Liu, et al., Endocrinology 2006, 147, 5061.

For studies related to skin aging, see, e.g., WO 2004/076418; WO 2004/103320; and US 2008/0070883.

For studies related to arthritis, see, e.g., Chintalacharuvu, et. al., Arthritis a& Rheumatism 2007, 56, 1365; and WO 2008/036239.

### SUMMARY

This invention relates generally to imidazo[1,2-a]pyridine-based modulators of Liver X receptors (LXRs) and related methods.

In one aspect, this invention features a compound having formula (I):

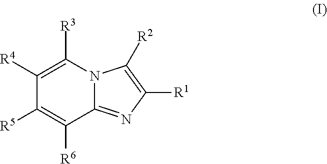

in which:

R¹ is:

(i) hydrogen; or

(ii) C₁-C₆ alkyl or C₁-C₆ haloalkyl, each of which is optionally substituted with from 1-10 R^{a}; or

(iii) C₂-C₆ alkenyl or C₂-C₆ alkynyl, each of which is optionally substituted with from 1-10 R^{b}; or

(iv) C₃-C₁₀ cycloalkyl, C₃-C₁₀ cycloalkenyl, heterocyclyl including 3-10 atoms, heterocycloalkenyl including 3-10 atoms, C₇-C₁₁ aralkyl, or heteroaralkyl including 6-11 atoms, each of which is optionally substituted with from 1-10 R^{c}; or

(v) C₆-C₁₀ aryl or heteroaryl including 5-10 atoms, each of which is optionally substituted with from 1-10 R^{d}; or

(vi) halo;

R² is C₆-C₁₀ aryl or heteroaryl including 5-10 atoms, each of which is:

(i) substituted with 1 R⁷, and

(ii) optionally substituted with from 1-5 R^{e}; wherein:

R⁷ is WA, wherein:

W is a bond; —O—; —NR⁸—; C₁₋₆ alkylene, C₂₋₆ alkenylene, or C₂₋₆ alkynylene; —W¹(C₁₋₆ alkylene)-; or —(C₁₋₆ alkylene)W¹—;

W¹ is independently —O— or —NR⁸—;

R⁸ is hydrogen or C₁-C₆ alkyl;

A at each occurrence is, independently, C₆-C₁₀ aryl or heteroaryl including 5-10 atoms, each of which is:

(i) substituted with 1 R⁹, and

(ii) optionally further substituted with from 1-5 R^{g};

R⁹ is:

(i) —W²—S(O)ₙR¹⁰ or —W²—S(O)ₙNR¹¹R¹²; or

(ii) —W²—C(O)OR¹³; or

(iii) —W²—C(O)NR¹¹R¹²; or

(iv) C₁-C₁₂ alkyl or C₁-C₁₂ haloalkyl, each of which is:
(a) substituted with 1 R^{h}, and
(b) optionally further substituted with from 1-5 R^{a};

or

(v) —NR¹⁴R¹⁵;

wherein:

W² at each occurrence is, independently, a bond; C₁₋₆ alkylene; C₂₋₆ alkenylene; C₂₋₆ alkynylene; C₃₋₆ cycloalkylene; —O(C₁₋₆ alkylene)-; or —NR⁸(C₁₋₆ alkylene)-;

n at each occurrence is, independently, 1 or 2;

R¹⁰ at each occurrence is, independently:

(i) C₁-C₆ alkyl or C₁-C₆ haloalkyl, each of which is optionally substituted with from 1-5 R^{a}; or

(ii) C₂-C₆ alkenyl or C₂-C₆ alkynyl, each of which is optionally substituted with from 1-5 R^{b}; or

(iii) C₃-C₁₀ cycloalkyl, C₃-C₁₀ cycloalkenyl, C₇-C₁₁ aralkyl, or heteroaralkyl including 6-11 atoms, each of which is optionally substituted with from 1-5 R^{c}; or

(iv) C₆-C₁₀ aryl or heteroaryl including 5-10 atoms, each of which is optionally substituted with from 1-5 R^{d};

R¹¹ and R¹² are each, independently:

(i) hydrogen; or

(ii)-(v) R¹⁰ (in which R¹⁰ is as defined above); or

(vi) heterocyclyl including 3-10 atoms or a heterocycloalkenyl including 3-10 atoms, each of which is optionally substituted with from 1-5 R^{c}; or

R¹¹ and R¹² together with the nitrogen atom to which they are attached form a heterocyclyl including 3-10 atoms or a heterocycloalkenyl including 3-10 atoms, each of which is optionally substituted with from 1-5 R^{c};

R¹³ is:

(i) hydrogen;

(ii)-(v) R¹⁰ (in which R¹⁰ is as defined above);

one of R¹⁴ and R¹⁵ is hydrogen or C₁-C₃ alkyl; and the other of R¹⁴ and R¹⁵ is:

(i) —S(O)ₙR¹⁰; or

(ii) —C(O)OR¹³; or

(iii) —C(O)NR¹¹R¹²; or

(iv) C₁-C₁₂ alkyl or C₁-C₁₂ haloalkyl, each of which is:
(a) substituted with 1 R^{h}, and
(b) optionally further substituted with from 1-5 R^{a};

each of R³, R⁴, and R⁵ is, independently:

(i) hydrogen; or

(ii) halo; or

(iii) C₁-C₆ alkyl or C₁-C₆ haloalkyl, each of which is optionally substituted with from 1-3 R^{a};

R⁶ is:

(i) hydrogen; or

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano;

R^{a} at each occurrence is, independently:

(i) NR^{m}Rⁿ; hydroxy; C₁-C₆ alkoxy or C₁-C₆ haloalkoxy; C₆-C₁₀ aryloxy or heteroaryloxy including 5-10 atoms, each of which is optionally substituted with from 1-5 R^{d}; C₇-C₁₁ aralkoxy, heteroaralkoxy including 6-11 atoms, C₃-C₁₁ cycloalkoxy, C₃-C₁₁ cycloalkenyloxy, heterocyclyloxy including 3-10 atoms, or heterocycloalkenyloxy including 3-10 atoms, each of which is optionally substituted with from 1-5 R^{c}; or cyano; or

(ii) C₃-C₁₀ cycloalkyl, C₃-C₁₀ cycloalkenyl, heterocyclyl including 3-10 atoms, or heterocycloalkenyl including 3-10 atoms, each of which is optionally substituted with from 1-5 R^{c};

R^{b} at each occurrence is, independently:

(i) halo; NR^{m}Rⁿ; hydroxy; C₁-C₆ alkoxy or C₁-C₆ haloalkoxy; C₆-C₁₀ aryloxy or heteroaryloxy including 5-10 atoms, each of which is optionally substituted with from 1-5 R^{d}; C₇-C₁₁ aralkoxy, heteroaralkoxy including 6-11 atoms, C₃-C₁₀ cycloalkoxy, C₃-C₁₀ cycloalkenyloxy, heterocyclyloxy including 3-10 atoms, or heterocycloalkenyloxy including 3-10 atoms, each of which is optionally substituted with from 1-5 R^{c};

(ii) C₃-C₁₀ cycloalkyl, C₃-C₁₀ cycloalkenyl, heterocyclyl including 3-10 atoms, or heterocycloalkenyl including 3-10 atoms, each of which is optionally substituted with from 1-5 R^{c}; or

(iii) C₆-C₁₀ aryl or heteroaryl including 5-10 atoms, each of which is optionally substituted with from 1-5 R^{d};

R^{c} at each occurrence is, independently:

(i) halo; NR^{m}Rⁿ; hydroxy; C₁-C₆ alkoxy or C₁-C₆ haloalkoxy; or

(ii) C₁-C₆ alkyl or C₁-C₆ haloalkyl, each of which is optionally substituted with from 1-5 R^{a}; or

(iii) C₂-C₆ alkenyl or C₂-C₆ alkynyl, each of which is optionally substituted with from 1-5 R^{b};

R^{d} at each occurrence is, independently:

(i) halo; NR^{m}Rⁿ; hydroxy; C₁-C₆ alkoxy or C₁-C₆ haloalkoxy; or cyano; or

(ii) C₁-C₆ alkyl or C₁-C₆ haloalkyl, each of which is optionally substituted with from 1-5 R^{a}; or

(iii) C₂-C₆ alkenyl or C₂-C₆ alkynyl, each of which is optionally substituted with from 1-5 R^{b};

R^{e} at each occurrence is, independently, C₁-C₆ alkyl; C₁-C₆ haloalkyl; halo; hydroxyl; NR^{m}Rⁿ; C₁-C₆ haloalkoxy; or cyano;

R^{g} at each occurrence is, independently:

(i) halo; NR^{m}Rⁿ; hydroxy; C₁-C₆ alkoxy or C₁-C₆ haloalkoxy; cyano; or

(ii) C₁-C₆ alkyl or C₁-C₆ haloalkyl;

R^{h} at each occurrence is, independently, hydroxyl, C₁-C₆ alkoxy, or C₁-C₆ haloalkoxy; C₃-C₁₀ cycloalkoxy or C₃-C₁₀ cycloalkenyloxy, each of which is optionally substituted with from 1-5 R^{c}; or C₆-C₁₀ aryloxy or heteroaryloxy including 5-10 atoms, each of which is optionally substituted with from 1-5 R^{d};

each of R^{m} and Rⁿ at each occurrence is, independently, hydrogen, C₁-C₆ alkyl, or C₁-C₆ haloalkyl;

or an N-oxide and/or salt (e.g., a pharmaceutically acceptable salt) thereof.

In one aspect, this invention features a compound having formula (I), in which R¹, R², R³, R⁴, R⁵, R⁷, R⁸, R⁹, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In one aspect, this invention features a compound having formula (I), in which R¹, R², R³, R⁴, R⁵, R⁶, R⁷, R⁸, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁹ is:

(i) —W²—S(O)ₙR¹⁰ or —W²—S(O)ₙNR¹¹R¹²; or

(iii) —W²—C(O)NR¹¹R¹²; or

(iv) C₁-C₁₂ alkyl or C₁-C₁₂ haloalkyl, each of which is:
(a) substituted with from 1 R^{h}, and
(b) optionally further substituted with from 1-5 R^{a};

or

(v) —NR¹⁴R¹⁵.

In certain embodiments:

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In one aspect, this invention features a compound having formula (I), in which R¹, R², R³, R⁴, R⁵, R⁶, R⁷, R⁸, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁹ is:

(i) —W²—S(O)ₙR¹⁰ or —W²—S(O)ₙNR¹¹R¹².

In certain embodiments:

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In another aspect, this invention features a compound having formula (I), in which R¹, R², R³, R⁴, R⁵, R⁶, R⁷, R⁸, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁹ is:

(i)—W²—S(O)ₙR¹⁰ or—W²—(O)ₙNR¹¹R¹²; or

(iv) C₁-C₁₂ alkyl or C₁-C₁₂ haloalkyl, each of which is:
(a) substituted with from 1 R^{h}, and
(b) optionally further substituted with from 1-5 R^{a};

or

(v) —NR¹⁴R¹⁵.

In certain embodiments:

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In one aspect, this invention features a compound having formula (I), in which R¹, R², R³, R⁴, R⁵, R⁶, R⁷, R⁸, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁹ is (ii) —W²—C(O)OR¹³.

In certain embodiments:

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In another aspect, this invention features a compound having formula (I), in which R¹, R², R³, R⁴, R⁵, R⁶, R⁷, R⁸, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁹ is (iii) —W²—C(O)NR¹¹R¹².

In certain embodiments:

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In a further aspect, this invention features a compound having formula (I), in which R¹, R², R³, R⁴, R⁵, R⁶, R⁷, R⁸, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁹ at each occurrence is, independently:

(iv) C₁-C₁₂ alkyl or C₁-C₁₂ haloalkyl, each of which is:
(a) substituted with from 1 R^{h}, and
(b) optionally further substituted with from 1-5 R^{a}.

In certain embodiments:

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In one aspect, this invention features a compound having formula (I), in which R¹, R², R³, R⁴, R⁵, R⁶, R⁷, R⁸, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein, and

R⁹ is (v)—NR¹⁴R¹⁵.

In certain embodiments:

R⁶ is:

(ii) halo; or

(iii) C₁-C₆ alkyl or C₁-C₆ haloalkyl, each of which is optionally substituted with from 1-3 R^{a}; or

(iv) nitro; C₁-C₆ alkoxy; C₁-C₆ haloalkoxy; C₁-C₆ thioalkoxy; C₁-C₆ thiohaloalkoxy; or cyano.

In certain embodiments, R¹ is other than halo.

In one aspect, this invention relates to any subgenera of formula (I) described herein.

In one aspect, this invention relates to any of the specific imidazo[1,2-a]pyridine compounds delineated herein. In some embodiments, the compound of formula (I) can be selected from the title compounds of Examples 9-30 and 36-142; or a pharmaceutically acceptable salt and/or N-oxide thereof.

In one aspect, this invention features a composition (e.g., a pharmaceutical composition), which includes a compound of formula (I) (including any subgenera or specific compounds thereof) or a salt (e.g., a pharmaceutically acceptable salt) or a prodrug thereof and a pharmaceutically acceptable adjuvant, carrier or diluent. In some embodiments, the composition can include an effective amount of the compound or the salt thereof. In some embodiments, the composition can further include an additional therapeutic agent.

In one aspect, this invention features a dosage form, which includes from about 0.05 milligrams to about 2,000 milligrams (e.g., from about 0.1 milligrams to about 1,000 milligrams, from about 0.1 milligrams to about 500 milligrams, from about 0.1 milligrams to about 250 milligrams, from about 0.1 milligrams to about 100 milligrams, from about 0.1 milligrams to about 50 milligrams, or from about 0.1 milligrams to about 25 milligrams) of formula (I) (including any subgenera or specific compounds thereof), or a salt (e.g., a pharmaceutically acceptable salt), or an N-oxide, or a prodrug thereof. The dosage form can further include a pharmaceutically acceptable carrier and/or an additional therapeutic agent.

The invention also relates generally to modulating (e.g., activating) LXRs with the imidazo[1,2-a]pyridine compounds described herein. In some embodiments, the methods can include, e.g., contacting an LXR in a sample (e.g., a tissue, a cell free assay medium, a cell-based assay medium) with a compound of formula (I) (including any subgenera or specific compounds thereof). In other embodiments, the methods can include administering a compound of formula (I) (including any subgenera or specific compounds thereof) to a subject (e.g., a mammal, e.g., a human, e.g., a human having or at risk of having one or more of the diseases or disorders described herein).

In one aspect, this invention also relates generally to methods of treating (e.g., controlling, ameliorating, alleviating, slowing the progression of, delaying the onset of, or reducing the risk of developing) or preventing one or more LXR-mediated diseases or disorders in a subject (e.g., a subject in need thereof). The methods include administering to the subject an effective amount of a compound of formula (I) (including any subgenera or specific compounds thereof) or a pharmaceutically acceptable salt or prodrug thereof. LXR-mediated diseases or disorders can include, e.g., cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging or connective tissue diseases.

In another aspect, this invention relates to methods of modulating (e.g., increasing) serum HDL cholesterol levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing) serum LDL cholesterol levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., increasing) reverse cholesterol transport in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing or inhibiting) cholesterol absorption in a subject (e.g., a subject in need thereof), which includes administering to the subject an effective amount of a compound of formula (I) (including any subgenera or specific compounds thereof) or a pharmaceutically acceptable salt or prodrug thereof.

In a further aspect, this invention relates to methods of preventing or treating a cardiovascular disease (e.g., acute coronary syndrome, restenosis, or coronary artery disease), which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating atherosclerosis and/or atherosclerotic lesions, which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating diabetes (e.g., type I diabetes or type II diabetes), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating Syndrome X, which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating obesity, which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating a lipid disorder (e.g., dyslipidemia, hyperlipidemia, hypertriglyceridemia, hypercholesterolemia, low HDL and/or high LDL), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating a cognitive disorder (e.g., Alzheimer's disease or dementia), which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating dementia, which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating Alzheimer's disease, which includes administering to a subject in need thereof an effective amount of a compound of formula (I) (including any subgenera or specific compounds thereof) or a pharmaceutically acceptable salt or prodrug thereof. In a further aspect, this invention relates to methods of preventing or treating an inflammatory disease (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating rheumatoid arthritis, which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating celiac, which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating thyroiditis, which includes administering to a subject in need thereof an effective amount of a compound of formula (I) (including any subgenera or specific compounds thereof) or a pharmaceutically acceptable salt or prodrug thereof.

In one aspect, this invention relates to methods of treating a connective tissue disease (e.g., osteoarthritis or tendonitis), which includes administering to a subject (e.g., a mammal, e.g., a human) in need thereof an effective amount of a compound of formula (I) (including any subgenera or specific compounds thereof) or a pharmaceutically acceptable salt or prodrug thereof. In embodiments, the compound of formula (I) inhibits (e.g., reduces or otherwise diminishes) cartilage degradation. In embodiments, the compound of formula (I) induces (e.g., increases or otherwise agments) cartilage regeneration. In embodiments, the compound of formula (I) inhibits (e.g., reduces or otherwise diminishes) cartilage degradation and induces (e.g., increases or otherwise agments) cartilage regeneration. In embodiments, the compound of formula (I) inhibits (e.g., reduces or otherwise diminishes) aggrecanase activity. In embodiments, the compound of formula (I) inhibits (e.g., reduces or otherwise diminishes) elaboration of pro-inflammatory cytokines in osteoarthritic lesions.

In another aspect, this invention relates to methods of treating or preventing skin aging, the method comprising administering (e.g., topically administering) to a subject (e.g., a mammal, e.g., a human) in need thereof an effective amount of a compound of formula (I) (including any subgenera or specific compounds thereof) or a pharmaceutically acceptable salt or prodrug thereof. In embodiments, the skin aging can be derived from chronological aging, photoaging, steroid-induced skin thinning, or a combination thereof.

The term “skin aging” includes conditions derived from intrinsic chronological aging (for example, deepened expression lines, reduction of skin thickness, inelasticity, and/or unblemished smooth surface), those derived from photoaging (for example, deep wrinkles, yellow and leathery surface, hardening of the skin, elastosis, roughness, dyspigmentations (age spots) and/or blotchy skin), and those derived from steroid-induced skin thinning. Accordingly, another aspect is a method of counteracting UV photodamage, which includes contacting a skin cell exposed to UV light with an effective amount of a compound of formula (I).

In some embodiments, the compound of formula (I) (including any subgenera or specific compounds thereof) does not substantially increase serum and/or hepatic triglyceride levels of the subject.

In some embodiments, the administered compound of formula (I) (including any subgenera or specific compounds thereof) can be an LXR agonist (e.g., an LXRα agonist or an LXRβ agonist, e.g., an LXRβ agonist).

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment and control of the diseases or disorders described herein.

In embodiments, any compound, composition, or method can also include any one or more of the following features (alone or in combination) and/or any one or more of the features (alone or in combination) delineated in the detailed description and/or in the claims.

R¹ can be hydrogen.

R¹ can be C₁-C₆ alkyl or C₁-C₄ haloalkyl (e.g., CF₃). For example, R¹ can be CH₃ (i.e., methyl), CH₃CH₂ (i.e., ethyl), or (CH₃)₂CH (i.e., isopropyl). As another example, R¹ can be CH₃, CH₃CH₂, (CH₃)₂CH, or (CH₃)₃C (i.e., tert-butyl).

R¹ can be C₁-C₆ alkyl or C₁-C₆ haloalkyl, each of which is optionally substituted with from 1-10 R^{a}. In certain embodiments, R¹ can be C₁-C₆ alkyl (e.g., CH₃CH₂ or (CH₃)₂CH). In other embodiments, R¹ can be C₁-C₆ (e.g., C₁-C₃, C₁) alkyl that is substituted with 1 R^{a}, in which R^{a} can be as defined anywhere herein. In certain embodiments, R^{a} can be NR^{m}Rⁿ; C₁-C₆ alkoxy; or heterocyclyl including 5 or 6 ring atoms, which is which is optionally substituted with from 1-5 (e.g., 1-4, 1-3, 1-2, or 1) R^{c}. For example, R^{a} can be heterocyclyl including 5 or 6 ring atoms, which is which is optionally substituted with from 1-5 (e.g., 1-4, 1-3, 1-2, or 1) R^{c}. For example, R^{a} can be optionally substituted pyrrolidinyl or thiazolidinyl (e.g., thiazolidinyl).

R¹ can be C₆-C₁₀ aryl or heteroaryl including 5-10 atoms, each of which is optionally substituted with from 1-5 (e.g., 1-4, 1-3, 1-2, or 1, e.g., 1) R^{d}. In embodiments, R¹ can be phenyl, which is optionally substituted with from 1-5 (e.g., 1-4, 1-3, 1-2, or 1, e.g., 1) R^{d}.

R¹ can be C₇-C₁₁ aralkyl, which is optionally substituted with from 1-5 (e.g., 1-4, 1-3, 1-2, or 1, e.g., 1) R^{c}. R¹ can be C₇-C₁₁ aralkyl or heteroaralkyl including 6-11 atoms, each of which is optionally substituted with from 1-5 (e.g., 1-4, 1-3, 1-2, or 1, e.g., 1) R^{c}. For example, R¹ can be benzyl, which is optionally substituted with from 1-5 (e.g., 1-4, 1-3, 1-2, or 1, e.g., 1 or 2) R^{c}.

R¹ can be C₃-C₈ cycloalkyl or heterocyclyl including 3-8 atoms, each of which is optionally substituted with from 1-3 R^{c}.

R² can be C₆-C₁₀ aryl, which is (a) substituted with from 1 R⁷; and (b) optionally substituted with from 1-2 R^{e}. R² can be C₆-C₁₀ aryl, which is (a) substituted with 1 R⁷; and (b) optionally substituted with from 1-4 R^{e}. In embodiments, R² can be phenyl, which is (a) substituted with 1 R⁷; and (b) optionally substituted with from 1 R^{e}. In other embodiments, R² can be phenyl, which is substituted with 1 R⁷.

R² can have formula (A-2):

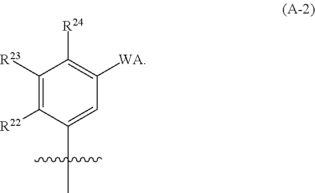

In some embodiments, each of R²², R²³, and R²⁴ can be, independently, hydrogen or R^{e}. In these and other embodiments related to formula (A-2), each of W, A, and R^{e} can be as defined anywhere herein.

In some embodiments, (i) each of R²², R²³, and R²⁴ is hydrogen; or (ii) one of R²², R²³, and R²⁴ is R^{e}, and the other two are hydrogen.

In certain embodiments, each of R²², R²³, and R²⁴ can be hydrogen. In other embodiments, one of R²², R²³, and R²⁴ can be R^{e}, and the other two are hydrogen. For example, R²² can be R^{e} (e.g., halo, e.g., chloro) and each of R²³ and R²⁴ can be hydrogen.

W can be —O—. W can be a bond. W can be —W¹(C₁₋₆ alkylene)-; in embodiments, W¹ can be —O—, and W can be, for example, —OCH₂—. W can be C₁₋₆ alkylene (e.g., —CH₂—).

A can be C₆-C₁₀ aryl, which is (a) substituted with from 1 R⁹; and (b) optionally substituted with from 1-4 R^{g}. In embodiments, A can be phenyl, which is (a) substituted with 1 R⁹; and (b) optionally substituted with from 1-4 R^{g}.

A can have formula (B-1):

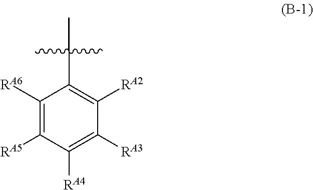

in which:

one of R^{A3} and R^{A4} is R⁹, the other of R^{A3} and R^{A4} is hydrogen; and

each of R^{A2}, R^{A5}, and R^{A6} is, independently, hydrogen or R^{g}. In these and other embodiments related to formula (B-1), each of R⁹ and R^{g} can be, independently, as defined anywhere herein.

R⁹ can be —W²—S(O)ₙR¹⁰. W² can be a bond. W² can be a bond, and n can be 2. R¹⁰ can be: C₁-C₆ alkyl or C₁-C₆ haloalkyl, each of which is optionally substituted with from 1-2 R^{a}. In embodiments, R¹⁰ can be C₁-C₆ alkyl, optionally substituted with from 1-2 R^{a}. For example, R¹⁰ can be C₁-C₅ alkyl (e.g., CH₃, CH₃CH₂, (CH₃)₂CH, e.g., CH₃). As another example, R¹⁰ can be C₂-C₆ alkyl substituted with 1 R^{a}. In embodiments, R^{a} can be hydroxyl, C₁-C₃ alkoxy, or NR^{m}Rⁿ.

R⁹ can be —W²—C(O)OR¹³.

R⁹ can be —W²—S(O)ₙNR¹¹R¹². W² can be a bond, and n can be 2. Each of R¹¹ and R¹² can be, independently: (i) hydrogen; or (ii) C₁-C₆ alkyl; or (iii) C₇-C₁₁ aralkyl, optionally substituted with from 1-5 R^{c}.

In some embodiments:

R² can be C₆-C₁₀ aryl, which is (a) substituted with 1 R⁷; and (b) optionally substituted with from 1-4 (e.g., 1-2) R^{e}; and

A can be C₆-C₁₀ aryl, which is (a) substituted with from 1 R^{g}; and (b) optionally substituted with from 1-4 R⁹. In these embodiments, each of R⁷, R⁹, R^{e}, and R^{g} can be, independently, as defined anywhere herein.

In certain embodiments:

R² can be phenyl, which is (a) substituted with 1 R⁷ (i.e., WA); and (b) optionally substituted with from 1 R^{e}; and

A can be phenyl, which is (a) substituted with 1 R⁹; and (b) optionally substituted with from 1-4 R⁹. In these embodiments, each of R⁷, R⁹, R^{e}, and R^{g} can be, independently, as defined anywhere herein.

R² can have formula (C-1):

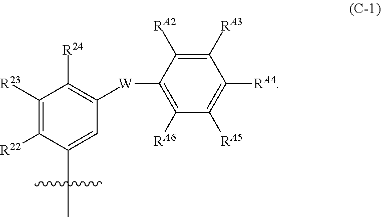

In some embodiments:

each of R²², R²³, and R²⁴ can be, independently, hydrogen or R^{e};

and

one of R^{A2}, R^{A3}, R^{A4}, R^{A5}, and R^{A6} can be R⁹, and the others are each, independently, hydrogen or R^{g}.

In some embodiments:

(i) each of R²², R²³, and R²⁴ can be hydrogen; or

(ii) one of R²², R²³, and R²⁴ can be R^{e}, and the other two are hydrogen;

and

one of R^{A2}, R^{A3}, R^{A4}, R^{A5}, and R^{A6} can be R⁹, and the others are each, independently, hydrogen or R^{g}.

In these and other embodiments related to formula (C-1), each of W, R⁹, R^{e} and R^{g} can be, independently, as defined anywhere herein.

Embodiments can include, for example, one or more of the following features (and/or any one or more other features described anywhere herein).

In certain embodiments, each of R²², R²³, and R²⁴ can be hydrogen. In other embodiments, one of R²², R²³, and R²⁴ can be R^{e}, and the other two are hydrogen. For example, R²² can be R^{e} (e.g., halo, e.g., chloro) and each of R²³ and R²⁴ can be hydrogen.

W can be —O—. W can be a bond. W is —W¹(C₁₋₆ alkylene)-; in embodiments, W¹ can be —O—, and W can be, for example, —OCH₂—. W can be C₁₋₆ alkylene (e.g., —CH₂—). W can be a bond, C₁₋₆ alkylene (e.g., —CH₂—), or —W¹(C₁₋₆ alkylene)- (e.g., —OCH₂—).

R⁹ can be —W²—S(O)ₙR¹⁰ or —W²—S(O)ₙNR¹¹R¹².

One of R^{A3} and R^{A4} can be R⁹, and the other of R^{A3} and R^{A4} can be hydrogen; and each of R^{A2}, R^{A5}, and R^{A6} can be, independently, hydrogen or R^{g}.

In certain embodiments, R^{A3} is —W²—S(O)ₙR¹⁰. Each of R^{A2}, R^{A5}, and R^{A6} can be hydrogen. W² can be a bond. n can be 2. R¹⁰ can be C₁-C₆ alkyl, optionally substituted with from 1-2 R^{a}. In embodiments, R¹⁰ can be C₁-C₅ alkyl (e.g., CH₃, CH₃CH₂, (CH₃)₂CH, e.g., CH₃). R¹⁰ can be C₂-C₆ alkyl substituted with 1 R^{a}. In embodiments, R^{a} can be hydroxyl, C₁-C₃ alkoxy, or NR^{m}Rⁿ. R^{A5} can be hydrogen or R^{g} (e.g., R^{g}), and each of R^{A2} and R^{A6} can be hydrogen.

In certain embodiments, R^{A4} can be —W²—C(O)OR¹³. R¹³ can be hydrogen. R¹³ can be C₁-C₃ alkyl. W² can be C₁-C₃ alkylene (e.g., CH₂). W² can be a bond. Each of R^{A2}, R^{A5}, and R^{A6} can be hydrogen.

One of R^{A3} and R^{A4} can be R⁹, and the other of R^{A3} and R^{A4} can be hydrogen; and each of R^{A2}, R^{A5}, and R^{A6} can be, independently, hydrogen or R^{g}. Each of R^{A2}, R^{A5}, and R^{A6} can be hydrogen. R^{A5} can be R^{g} (e.g., halo, e.g., fluoro), and each of R^{A2} and R^{A6} can be hydrogen.

In certain embodiments, R^{A3} can be R⁹, and R^{A4} can be hydrogen. In embodiments, R⁹ can be —W²—S(O)ₙR¹⁰ or —W²—S(O)ₙNR¹¹R¹²

For example, when R^{A3} is R⁹, R⁹ can be —W²—S(O)ₙR¹⁰. W² can be a bond; n can be 2; and R¹⁰ can be C₁-C₆ alkyl, optionally substituted with from 1-2 R^{a}. In embodiments, R¹⁰ can be C₁-C₅ alkyl (e.g., CH₃, CH₃CH₂, (CH₃)₂CH, e.g., CH₃). R¹⁰ can be C₂-C₆ alkyl substituted with 1 R^{a}. In embodiments, R^{a} can be hydroxyl, C₁-C₃ alkoxy, or NR^{m}Rⁿ. R^{a} can also be cyano. R^{A5} can be hydrogen or R^{g} (e.g., R^{g}), and each of R^{A2} and R^{A6} can be hydrogen.

As another example, when R^{A3} is R⁹, R⁹ can be —W²—S(O)ₙNR¹¹R¹². W² can be a bond; n can be 2, and each of R¹¹ and R¹² is, independently: (i) hydrogen; or (ii) C₁-C₆ alkyl; or (iii) C₇-C₁₁ aralkyl, optionally substituted with from 1-5 R^{c}.

Each of R³, R⁴, and R⁵ can be, independently: (i) hydrogen; or (ii) halo. Each of R³, R⁴, and R⁵ can be hydrogen.

R⁶ can be: (ii) halo; or (iii) C₁-C₆ alkyl or C₁-C₆ haloalkyl, each of which is optionally substituted with from 1-3 R^{e}; or (iv) cyano.

R⁶ can be C₁-C₆ haloalkyl. In certain embodiments, R⁶ can be C₁-C₃ perfluoroalkyl (e.g., CF₃).

R⁶ can be halo (e.g., chloro).

R⁶ can be cyano.

One or more of R¹, R³, R⁴, R⁵, and R⁶ (e.g., R¹ and/or R⁶) can be a substituent other than hydrogen.

The compound can have formula (VI):

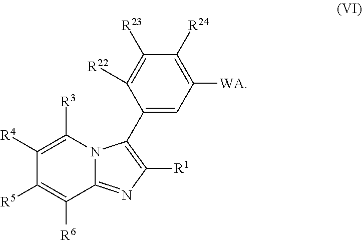

In some embodiments:

R¹ is:

(i) hydrogen; or

(ii) C₁-C₃ alkyl or C₁-C₃ haloalkyl; or

(iii) phenyl or heteroaryl including 5-6 atoms, each of which is optionally substituted with from 1-5 R^{d}; or

(iv) C₇-C₁₁ aralkyl or heteroaralkyl including 6-11 atoms, each of which is optionally substituted with from 1-3 R^{c};

R⁶ is:

(ii) halo; or

(iii) C₁-C₃ alkyl or C₁-C₃ haloalkyl, each of which is optionally substituted with from 1-3 R^{a}; or

(iv) cyano; and

each of R²², R²³, and R²⁴ is, independently, hydrogen or R^{e} (as defined anywhere herein).

Embodiments can include one or more of the following features (and/or any one or more other features described anywhere herein).

R¹ can be hydrogen. R¹ can be CH₃, CH₃CH₂ or (CH₃)₂CH. R¹ can be phenyl, which is optionally substituted with from 1-5 R^{d}. R¹ can be benzyl, which is optionally substituted with from 1-5 R^{d}.

W can be —O—. W can be a bond. W can be —OCH₂—.

A can have formula (B-1), in which one of R^{A3} and R^{A4} is R⁹, and the other of R^{A3} and R^{A4} is hydrogen; and each of R^{A2}, R^{A5}, and R^{A6} is, independently, hydrogen or R^{g}. R^{A3} can be —W²—S(O)ₙR¹⁰, in which W² can be a bond, and n can be 2. R¹⁰ can be C₁-C₆ alkyl, optionally substituted with from 1-2 R^{a}. R¹⁰ can be CH₃, CH₂CH₃, or isopropyl. R¹⁰ can be C₂-C₈ alkyl substituted with 1 R^{a}. R^{a} can be hydroxyl or C₁-C₃ alkoxy. R^{A5} can be hydrogen or R^{g}, and each of R^{A2} and R^{A6} can be hydrogen. R^{A4} is —W²—C(O)OR¹³. R¹³ can be hydrogen or C₁-C₃ alkyl. W² can be CH₂. Each of R^{A2}, R^{A5}, and R^{A6} can be hydrogen. Each of R³, R⁴, and R⁵ can be hydrogen. Each of R²², R²³, and R²⁴ can be hydrogen. One of R²², R²³, and R²⁴ can be R^{e}, and the other two are hydrogen. For example, R²² can be R^{e} (e.g., halo, e.g., chloro) and each of R²³ and R²⁴ can be hydrogen. R⁶ can be CF₃. R⁶ can be chloro.

In some embodiments, the following definitions can apply to compounds having formula (VI):

R¹ is:

(i) C₁-C₆ alkyl or C₁-C₆ haloalkyl, each of which is optionally substituted with from 1-10 R^{a}; or

(ii) phenyl or heteroaryl including 5-6 atoms, each of which is optionally substituted with from 1-5 R^{d}; or

(iii) C₇-C₁₁ aralkyl or heteroaralkyl including 6-11 atoms, each of which is optionally substituted with from 1-3 R^{c};

each of R³, R⁴, and R⁵ is hydrogen;

R⁶ is:

(ii) halo; or

(iii) C₁-C₃ alkyl or C₁-C₃ haloalkyl, each of which is optionally substituted with from 1-3 R^{a}; or

(iv) cyano;

each of R²², R²³, and R²⁴ is hydrogen; or one of R²², R²³, and R²⁴ is R^{e}, and the other two are hydrogen;

W is a bond, —O—, —OCH₂—, or —CH₂—;

A has formula (B-1), wherein one of R^{A3} and R^{A4} is R⁹, and the other of R^{A3} and R^{A4} is hydrogen; and each of R^{A2}, R^{A5}, and R^{A6} is, independently, hydrogen or R^{g}; and

R⁹ is —W²—S(O)ₙR¹² or —W²—S(O)ₙNR¹¹R¹².

Embodiments can include any one or more of the features described anywhere herein.

The term “mammal” includes organisms, which include mice, rats, cows, sheep, pigs, rabbits, goats, horses, monkeys, dogs, cats, and humans.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/Kg to about 1000 mg/Kg, (e.g., from about 0.1 mg/Kg to about 100 mg/Kg, from about 1 mg/Kg to about 100 mg/Kg). Effective doses will also vary depending on route of administration, as well as the possibility of co-usage with other agents.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₂₀ alkyl indicates that the group may have from 1 to 20 (inclusive) carbon atoms in it. Any atom can be optionally substituted, e.g., by one or more substitutents. Examples of alkyl groups include without limitation methyl, ethyl, n-propyl, isopropyl, and tert-butyl.

The term “cycloalkyl” refers to saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl groups can contain fused rings. Fused rings are rings that share a common carbon atom. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The terms “alkylene,” “alkenylene,” “alkynylene,” and “cycloalkylene” refer to divalent straight chain or branched chain alkyl (e.g., —CH₂—), alkenyl (e.g., —CH═CH—), alkynyl (e.g., —C≡C—); or cycloalkyl moieties, respectively.

The term “haloalkyl” refers to an alkyl group, in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26, etc. hydrogen atoms) on a alkyl group can be replaced by more than one halogen (e.g., 2, 3, 4, 5, 6, 7, 8, 9, 10, 11, 12, 13, 14, 15, 16, 17, 18, 19, 20, 21, 22, 23, 24, 25, 26, etc. halogen atoms). In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (e.g., perhaloalkyl, e.g., perfluoroalkyl, such as trifluoromethyl). Any atom can be optionally substituted, e.g., by one or more substituents.

The term “aralkyl” refers to an alkyl moiety in which an alkyl hydrogen atom is replaced by an aryl group. One of the carbons of the alkyl moiety serves as the point of attachment of the aralkyl group to another moiety. Aralkyl includes groups in which more than one hydrogen atom on an alkyl moiety has been replaced by an aryl group. Any ring or chain atom can be optionally substituted e.g., by one or more substituents. Non-limiting examples of “aralkyl” include benzyl, 2-phenylethyl, 3-phenylpropyl, benzhydryl (diphenylmethyl), and trityl (triphenylmethyl) groups.

The term “heteroaralkyl” refers to an alkyl moiety in which an alkyl hydrogen atom is replaced by a heteroaryl group. One of the carbons of the alkyl moiety serves as the point of attachment of the aralkyl group to another moiety. Heteroaralkyl includes groups in which more than one hydrogen atom on an alkyl moiety has been replaced by a heteroaryl group. Any ring or chain atom can be optionally substituted e.g., by one or more substituents. Heteroaralkyl can include, for example, 2-pyridylethyl.

The term “alkenyl” refers to a straight or branched hydrocarbon chain containing 2-20 carbon atoms and having one or more double bonds. Any atom can be optionally substituted, e.g., by one or more substituents. Alkenyl groups can include, e.g., allyl, 1-butenyl, 2-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical. The term “mercapto” refers to an SH radical. The term “thioalkoxy” refers to an —S-alkyl radical. The terms “aryloxy” and “heteroaryloxy” refer to an —O-aryl radical and —O-heteroaryl radical, respectively. The terms “thioaryloxy” and “thioheteroaryloxy” refer to an —S-aryl radical and —S-heteroaryl radical, respectively.

The terms “aralkoxy” and “heteroaralkoxy” refer to an —O-aralkyl radical and —O-heteroaralkyl radical, respectively. The terms “thioaralkoxy” and “thioheteroaralkoxy” refer to an —S-aralkyl radical and —S-heteroaralkyl radical, respectively. The term “cycloalkoxy” refers to an —O-cycloalkyl radical. The terms “cycloalkenyloxy” and “heterocycloalkenyloxy” refer to an —O-cycloalkenyl radical and —O-heterocycloalkenyl radical, respectively. The term “heterocyclyloxy” refers to an —O-heterocyclyl radical. The term “thiocycloalkoxy” refers to an —S-cycloalkyl radical. The terms “thiocycloalkenyloxy” and “thioheterocycloalkenyloxy” refer to an —S-cycloalkenyl radical and —S-heterocycloalkenyl radical, respectively. The term “thioheterocyclyloxy” refers to an —S-heterocyclyl radical.

The term “heterocyclyl” refers to a saturated monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂). Thus, a heterocyclyl ring includes carbon atoms and 1-4, 1-8, or 1-10 heteroatoms selected from N, O, or S if monocyclic, bicyclic, or tricyclic, respectively. A ring heteroatom or ring carbon is the point of attachment of the heterocyclyl substituent to another moiety. Any atom can be optionally substituted, e.g., by one or more substituents. The heterocyclyl groups can contain fused rings. Fused rings are rings that share a common carbon or nitrogen atom. Heterocyclyl groups can include, e.g., tetrahydrofuryl, tetrahydropyranyl, piperidyl (piperidino), piperazinyl, morpholinyl (morpholino), pyrrolinyl, and pyrrolidinyl.

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents. The cycloalkenyl groups can contain fused rings. Fused rings are rings that share a common carbon atom. Cycloalkenyl moieties can include, e.g., cyclohexenyl, cyclohexadienyl, or norbornenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). A ring carbon (e.g., saturated or unsaturated) or heteroatom is the point of attachment of the heterocycloalkenyl substituent. Any atom can be optionally substituted, e.g., by one or more substituents. The heterocycloalkenyl groups can contain fused rings. Fused rings are rings that share a common carbon or nitrogen atom. Heterocycloalkenyl groups can include, e.g., tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “aryl” refers to a fully unsaturated, aromatic monocyclic, bicyclic, or tricyclic, hydrocarbon ring system, wherein any ring atom can be optionally substituted, e.g., by one or more substituents. Aryl groups can contain fused rings. Fused rings are rings that share a common carbon atom. Aryl moieties can include, e.g., phenyl, naphthyl, anthracenyl, and pyrenyl.

The term “heteroaryl” refers to a fully unsaturated, aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms independently selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). Any atom can be optionally substituted, e.g., by one or more substituents. Heteroaryl groups can contain fused rings. Fused rings are rings that share a common carbon or nitrogen atom. Heteroaryl groups can include, e.g., pyridyl, thienyl, furyl (furanyl), imidazolyl, indolyl, isoquinolyl, quinolyl and pyrrolyl.

The descriptor C(O) refers to a carbon atom that is doubly bonded to oxygen.

The term “substituent” refers to a group “substituted” on, e.g., an alkyl, haloalkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituent(s) (e.g., R^{d}) on a group are independently any one single, or any combination of two or more of the permissible atoms or groups of atoms delineated for that substituent. In another aspect, a substituent may itself be substituted with any one of the above substituents.

In general, when a definition for a particular variable includes both hydrogen and non-hydrogen (halo, alkyl, aryl, etc.) possibilities, the term “substituent(s) other than hydrogen” refers collectively to the non-hydrogen possibilities for that particular variable.

Descriptors such as “C₁-C₆ alkyl which is optionally substituted with from 1-2 R^{a}” (and the like) is intended to include as alternatives both unsubstituted C₁-C₆ alkyl and C₁-C₆ alkyl that is substituted with from 1-2 R^{a}. The use of a substituent (radical) prefix names such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo.

In some embodiments, the compounds have agonist activity for genes involved with HDL production and cholesterol efflux (e.g., ABCA1) and antagonist activity for genes involved with triglyceride synthesis (e.g., SREBP-1c).

The details of one or more embodiments of the invention are set forth in the description below. Other features and advantages of the invention will be apparent from the description and from the claims.

### DETAILED DESCRIPTION

This invention relates generally to imidazo[1,2-a]pyridine-based modulators of Liver X receptors (LXRs) and related methods.

The imidazo[1,2-a]pyridine-based LXR modulators have the general formula (I):

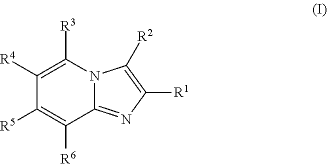

in which R¹, R², R³, R⁴, R⁵, R⁶, R⁷, R⁸, R⁹, R¹⁰, R¹¹, R¹², R¹³, R¹⁴, R¹⁵, W, W¹, W², A, R^{a}, R^{b}, R^{c}, R^{d}, R^{e}, R^{g}, R^{h}, R^{m}, Rⁿ, and n, can be, independently, as defined anywhere herein.

For ease of exposition, it is understood that where in this specification (including the claims), a group is defined by “as defined anywhere herein” (or the like), the definitions for that particular group include the first occurring and broadest generic definition as well as any sub-generic and specific definitions delineated anywhere in this specification.

Variable R¹

In some embodiments, R¹ can be:

(1-i) hydrogen; or

(1-ii) C₁-C₆ (e.g., C₁-C₃) alkyl or C₁-C₆ (e.g., C₁-C₄ or C₁-C₃) haloalkyl, each of which is optionally substituted with from 1-10 (e.g., 1-5, 1-4, 1-3, 1-2, 1) R^{a}; or

(1-iv) C₃-C₁₀ (e.g., C₃-C₈ or C₃-C₆) cycloalkyl, C₃-C₁₀ (e.g., C₃-Cₛ or C₃-C₆) cycloalkenyl, heterocyclyl including 3-10 (e.g., 3-8 or 3-6) atoms, heterocycloalkenyl including 3-10 (e.g., 3-8 or 3-6) atoms, C₇-C₁₁ (e.g., C₇-C₁₀) aralkyl, or heteroaralkyl including 6-11 (e.g., 6-10) atoms, each of which is optionally substituted with from 1-10 (e.g., 1-5, 1-4, 1-3, 1-2, 1) R^{c}; or

(1-v) C₆-C₁₀ (e.g., phenyl) aryl or heteroaryl including 5-10 (e.g., 5-6) atoms, each of which is optionally substituted with from 1-10 (e.g., 1-5, 1-4, 1-3, 1-2, 1) R^{d}.

In some embodiments, R¹ can be:

(1-i) hydrogen; or

(1-ii) C₁-C₆ (e.g., C₁-C₃) alkyl or C₁-C₆ (e.g., C₁-C₄) haloalkyl, each of which is optionally substituted with from 1-10 (e.g., 1-5, 1-4, 1-3, 1-2, 1) R^{a}; or

(1-iv′) C₇-C₁₁ (e.g., C₇-C₁₀) aralkyl, or heteroaralkyl including 6-11 (e.g., 6-10) atoms, each of which is optionally substituted with from 1-10 (e.g., 1-5, 1-4, 1-3, 1-2, 1) R^{c}; or

(1-v) C₆-C₁₀ (e.g., phenyl) aryl or heteroaryl including 5-10 (e.g., 5-6) atoms, each of which is optionally substituted with from 1-10 (e.g., 1-5, 1-4, 1-3, 1-2, 1) R^{d}.

In some embodiments, R¹ can be any one of: (1-i), (1-ii), (1-iv), (1-iv′), and (1-v). In certain embodiments, R¹ can be hydrogen. In other embodiments, R¹ can be a substituent other than hydrogen.

In some embodiments, R¹ can be any two of: (1-i), (1-ii), (1-iv), (1-iv′), and (1-v). In certain embodiments, R¹ can be hydrogen and any one of (1-ii), (1-iv), (1-iv′), and (1-v). In other embodiments, R¹ can be any two of (1-ii), (1-iv), (1-iv′), and (1-v), e.g., R¹ can be (1-ii) and (1-iv′).

In some embodiments, R¹ can be any three of: (1-i), (1-ii), (1-iv), (1-iv′), and (1-v). In certain embodiments, R¹ can be hydrogen and any two of (1-ii), (1-iv), (1-iv′), and (1-v), e.g., R¹ can be (1-ii) and (1-iv′). In other embodiments, R¹ can be any three of (1-11), (1-iv), (1-iv′), and (1-v), e.g., (1-ii), (1-iv′), and (1-v).

In embodiments, R¹ can be C₁-C₆ (e.g., C₁-C₃) alkyl. For example, R¹ can be methyl (CH₃), ethyl (CH₂CH₃), or isopropyl (CH(CH₃)₂).

In embodiments, R¹ can be C₁-C₆ (e.g., C₁-C₃, C₁) alkyl that is substituted with 1 R^{a}, in which R^{a} can be as defined anywhere herein. In certain embodiments, R^{a} can be NR^{m}Rⁿ; C₁-C₆ alkoxy; or heterocyclyl including 5 or 6 ring atoms, which is which is optionally substituted with from 1-5 (e.g., 1-4, 1-3, 1-2, or 1) R^{c}. For example, R^{a} can be heterocyclyl including 5 or 6 ring atoms, which is which is optionally substituted with from 1-5 (e.g., 1-4, 1-3, 1-2, or 1) R^{c}. For example, R^{a} can be optionally substituted pyrrolidinyl or thiazolidinyl (e.g., thiazolidinyl).

In embodiments, R¹ can be C₁-C₆ (e.g., C₁-C₄ or C₁-C₃) haloalkyl (e.g., perhaloalkyl). For example, R¹ can be CF₃.

In embodiments, R¹ can be C₇-C₁₁ (e.g., C₇-C₁₀) aralkyl, which is optionally substituted with from 1-5 (e.g., 1-4, 1-3, 1-2, 1) R^{c}. For example, R¹ can be benzyl or 2-phenylethyl, each of which is optionally substituted with from 1-5 (e.g., 1-4, 1-3, 1-2, 1) R^{c}.

In embodiments, R¹ can be heteroaralkyl including 6-10 atoms, which is optionally substituted with from 1-5 (e.g., 1-4, 1-3, 1-2, 1) R^{c}. In certain embodiments, the alkyl portion can be C₁-C₂ alkylene, and the heteroaryl portion can be thienyl, furyl, pyrrolyl, or pyridinyl, each of which is optionally substituted with from 1-5 (e.g., 1-4, 1-3, 1-2, 1) R^{c}.

In embodiments, R¹ can be C₆-C₁₀ aryl, which is optionally substituted with from 1-5 (e.g., 1-4, 1-3, 1-2, 1) R^{d}. For example, R¹ can be phenyl, which is optionally substituted with from 1-5 (e.g., 1-4, 1-3, 1-2, 1) R^{d}.

In embodiments, R¹ can be heteroaryl including 5-10 (e.g., 5-6) atoms, each of which is optionally substituted with from 1-5 (e.g., 1-4, 1-3, 1-2, 1) R^{d}. For example, R¹ can be thienyl, furyl, pyrrolyl, or pyridinyl, each of which is optionally substituted with from 1-5 (e.g., 1-4, 1-3, 1-2, 1) R^{d}.

In some embodiments, R¹ can be halo (e.g., chloro).

In other embodiments, R¹ can be other than halo.

Variable R²

In some embodiments, R² can be C₆-C₁₀ (e.g., phenyl) aryl, which is (i) substituted with 1 R⁷ and (ii) optionally substituted with from 1-4 (e.g., 1-3, 1-2, 1) R^{e}.

In some embodiments, when R² is aryl and substituted with R^{e}, each R^{e} can be independently of one another: halo (e.g., chloro); C₁-C₃ alkyl; C₁-C₃ haloalkyl (e.g., C₁-C₃ fluoroalkyl, e.g., 1-5 fluorines can be present; or C₁-C₃ perfluoroalkyl); CN; hydroxyl; NR^{m}Rⁿ (e.g., NH₂, monoalkylamino, or dialkylamino); C₁-C₃ alkoxy; C₁-C₃ haloalkoxy.

In certain embodiments, when R² is substituted with R^{e}, each R^{e} can be independently of one another: C₁-C₃ alkyl; C₁-C₃ haloalkyl, e.g., C₁-C₃ perfluoroalkyl; halo (e.g., chloro); or CN.

In certain embodiments, when R² is substituted with R^{e}, each R^{e} can be independently of one another: C₁-C₃ alkyl; C₁-C₃ haloalkyl, e.g., C₁-C₃ perfluoroalkyl; halo (e.g., chloro).

In certain embodiments, when R² is substituted with R^{e}, each R^{e} can be independently of one another halo (e.g., chloro).

In some embodiments, R² can be C₆-C₁₀ aryl, which is (i) substituted with 1 R⁷ and (ii) optionally substituted with from 1-4 (e.g., 1-3, 1-2, 1) R^{e}.

In some embodiments, R² can be C₆-C₁₀ aryl, which is (i) substituted with 1 or 2 R⁷ and (ii) optionally substituted with 1 or 2 R^{e}.

In certain embodiments, R² can be phenyl, which is (i) substituted with 1 R⁷ and (ii) optionally substituted with 1 or 2 (e.g., 1) R^{e} (e.g., halo, e.g., chloro). In other embodiments, R² can be phenyl, which is substituted with 1 R⁷. In these embodiments, R² can have formula (A), in which R⁷ (i.e., the moiety —WA) can be attached to a ring carbon that is ortho, meta, or para (e.g., meta) with respect to the ring carbon that connects the phenyl ring to the 3-position of the imidazo[1,2-a]pyridine ring, and R^{e}, when present can be connected to ring carbons that are not occupied by WA. For example, R² can have formula (A-1), in which R⁷ (WA) is attached to the ring carbon that is meta with respect to the ring carbon that connects the phenyl ring to the 3-position of the imidazo[1,2-a]pyridine ring in formula (I). As another example, R⁷ (WA) can be attached to the ring carbon that is para with respect to the ring carbon that connects the phenyl ring to the 3-position of the imidazo[1,2-a]pyridine ring in formula (I).

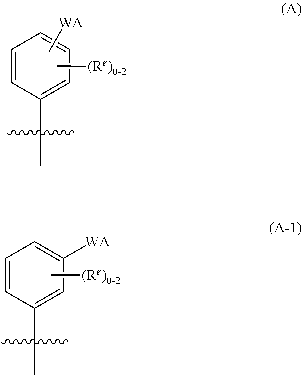

In certain embodiments, R² can have formula (A-2):

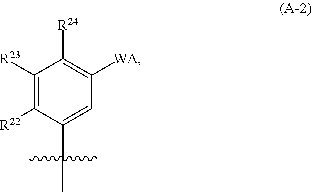

in which each of R²², R²³, and R²⁴ can be, independently of one another, hydrogen or R^{e}, in which R^{e} can be as defined anywhere herein. In embodiments, each of R²², R²³, and R²⁴ can be hydrogen; or one of R²², R²³, and R²⁴ can be R^{e}, and the other two are hydrogen. In these and other embodiments related to formula (A-2), each of W, A, and R^{e} can be as defined anywhere herein.

In embodiments, each of R²², R²³, and R²⁴ can be hydrogen.

In other embodiments, each of R²², R²³, and R²⁴ can be a substituent other than hydrogen. In still other embodiments, one or two of R²², R²³ and R²⁴ can be R^{e}, and the other(s) are hydrogen.

In certain embodiments, one of R²², R²³, and R²⁴ can be R^{e}, and the other two are hydrogen. In embodiments, R²² can be R^{e}, and each of R²³ and R²⁴ can be hydrogen. In certain embodiments, R^{e} can be: halo (e.g., chloro); C₁-C₃ alkyl; or C₁-C₃ haloalkyl (e.g., C₁-C₃ fluoroalkyl, e.g., 1-5 fluorines can be present; or C₁-C₃ perfluoroalkyl). In certain embodiments, R^{e} can be halo (e.g., chloro).

In some embodiments, R² can be heteroaryl including 5-10 (e.g., 5-6) atoms, which is (i) substituted with 1 R⁷ and (ii) optionally substituted with from 1-4 (e.g., 1-3, 1-2, 1) R^{e}.

In embodiments, when R² is heteroaryl and substituted with R^{e}, each R^{e} can be independently as defined anywhere herein. For example, each R^{e} can be independently of one another: C₁-C₃ alkyl; C₁-C₃ haloalkyl, e.g., C₁-C₃ perfluoroalkyl; halo (e.g., chloro); e.g., each R^{e} can be halo (e.g., chloro).

In some embodiments, R² can be heteroaryl including 5-10 atoms, which is (i) substituted with 1 R⁷ and (ii) optionally substituted with from 1-4 (e.g., 1-3, 1-2, 1) R^{e}.

In some embodiments, R² can be heteroaryl including 5-10 atoms, which is (i) substituted with 1 R⁷ and (ii) optionally substituted with 1 or 2 R^{e}.

In some embodiments, R² can be heteroaryl including 5-6 atoms, which is (i) substituted with 1 R⁷ and (ii) optionally substituted with 1 or 2 R^{e}.

In some embodiments, R² can be heteroaryl including 8-10 atoms, which is (i) substituted with 1 R⁷ and (ii) optionally substituted with 1 or 2 R^{e}.

In certain embodiments, R² can be pyridyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), each of which is (i) substituted with 1 R⁷ and (ii) optionally substituted with 1 or 2 R^{e}. For example, R² can be pyridyl substituted with 1 R⁷.

Variable W

In some embodiments, W can be —O—.

In some embodiments, W can be a bond.

In other embodiments, W can be —W¹(C₁₋₆ alkylene)-. In certain embodiments, W¹ can be —O—. For example, W can be —O(C₁₋₃ alkylene)- (e.g., —OCH₂—).

In some embodiments, W can be —NR⁸— (e.g., —NH—).

In some embodiments, W can be —(C₁₋₆ alkylene)W¹—. In certain embodiments, W¹ is —NR⁹—, in which R⁹ can be hydrogen; or W¹ can be —O—. In certain embodiments, W can be —(C₁₋₃ alkylene)NH— (e.g., —CH₂NH—). In certain embodiments, W can be —(C₁₋₃ alkylene)O— (e.g., —CH₂O—).

In still other embodiments, W can be C₂-C₄ alkenylene (e.g., —CH═CH—); C₂-C₄ alkynylene (e.g., —C≡C—); or C₁₋₃ alkylene (e.g., CH₂).

Variable A

In general, A is an aromatic or heteroaromatic ring system that is (a) substituted with one R⁹; and (b) optionally substituted with one or more R^{g}.

In some embodiments, A can be C₆-C₁₀ (e.g., phenyl) aryl, which is (a) substituted with 1 R⁹; and (b) optionally further substituted with from 1-5 (e.g., 1-4, 1-3, 1-2, 1, e.g., 1-2) R^{g}, in which R^{g} can be as defined anywhere herein.

In embodiments, when A is aryl and substituted with one or more R^{g}, each R^{g} can be independently of one another:

(i) halo; C₁-C₆ (e.g., C₁-C₃) alkoxy or C₁-C₆ (e.g., C₁-C₃) haloalkoxy; or cyano; or

(ii) C₁-C₆ (e.g., C₁-C₃) alkyl or C₁-C₆ (e.g., C₁-C₃) haloalkyl.

In certain embodiments, R^{g} is other than cyano.

In embodiments, when A is aryl and substituted with one or more R^{g}, each R^{g} can be independently of one another:
halo (e.g., chloro or fluoro); or
C₁-C₆ (e.g., C₁-C₃) haloalkoxy; or
C₁-C₆ (e.g., C₁-C₃) alkoxy; NR^{m}Rⁿ; or
cyano; or
C₁-C₆ (e.g., C₁-C₃) alkyl or C₁-C₆ (e.g., C₁-C₃) haloalkyl.

In certain embodiments, R^{g} is other than cyano.

In some embodiments, A can be C₆-C₁₀ aryl, which is (i) substituted with 1 R⁹ and (ii) optionally substituted with from 1-5 (e.g., 1-5, 1-4, 1-3, 1-2, 1, e.g., 1-2) R^{g}.

In some embodiments, A can be phenyl, which is (i) substituted with 1 R⁹ and (ii) optionally substituted with from 1-4 (e.g., 1-3, 1-2, 1) R^{g}.

In these embodiments, R⁹ can be attached to a ring carbon that is ortho, meta, or para (e.g., meta or para) with respect to the ring carbon that connects the phenyl ring to W.

In certain embodiments, A can have formula (B-1):

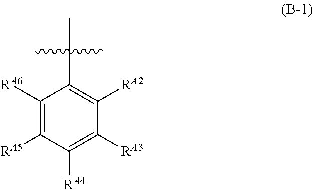

in which one of R^{A3} and R^{A4} is R⁹, the other of R^{A3} and R^{A4} and each of R^{A2}, R^{A5}, and R^{A6} is, independently, hydrogen or R^{g}. In these and other embodiments related to formula (B-1), each of R⁹ and R^{g} can be, independently, as defined anywhere herein.

In embodiments, one of R^{A3} and R^{A4} can be R⁹, the other of R^{A3} and R^{A4} can be hydrogen; and each of R^{A2}, R^{A5}, and R^{A6} can be, independently, hydrogen or R^{g}.

In certain embodiments, R^{A3} can be R⁹. For example, R^{A3} can be R⁹, R^{A4} can be hydrogen, and each of R^{A2}, R^{A5}, and R^{A6} can be hydrogen. As another example, R^{A3} can be R⁹; R^{A4} can be hydrogen; one of R^{A2}, R^{A5}, and R^{A6} (e.g., R^{A5}) can be R^{g} (e.g., halo) and the other two of R^{A2}, R^{A5}, and R^{A6} can be hydrogen.

In certain embodiments, R^{A4} can be R⁹. For example, R^{A4} can be R⁹, R^{A3} can be hydrogen, and each of R^{A2}, R^{A5}, and R^{A6} can be hydrogen. As another example, R^{A3} can be R⁹; R^{A4} can be hydrogen; one of R^{A2}, R^{A5}, and R^{A6} can be R^{g} (e.g., halo) and the other two of R^{A2}, R^{A5}, and R^{A6} can be hydrogen.

In some embodiments, A can be heteroaryl including 5-10 atoms, which is (a) substituted 1 R⁹; and (b) is optionally substituted with from 1-3 (e.g., 1-2, 1) R^{g}, in which R⁹ can be as defined anywhere herein.

In certain embodiments, A can be pyrrolyl, pyridyl, pyridyl-N-oxide, pyrazolyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), which is (i) substituted with 1 R⁹ and (ii) optionally substituted with 1-3 (e.g., 1-2, 1) R^{g}.

In certain embodiments, A can be pyrrolyl, pyridyl, pyrimidinyl, pyrazolyl, thienyl, furyl, quinolyl, oxazolyl, thiazolyl, imidazolyl, or isoxazolyl, each of which is (a) substituted with 1 R⁹; and (b) is optionally substituted with from 1-3 (e.g., 1-2, 1) R^{g}.

In certain embodiments, A can be pyridyl, pyrimidinyl, thienyl, furyl, oxazolyl, thiazolyl, imidazolyl, or isoxazolyl, each of which is (a) substituted with 1 R⁹; and (b) is optionally substituted with from 1-3 (e.g., 1-2, 1) R^{g}.

In certain embodiments, A can be pyridyl in which W is attached to the 2- or 3-position of the pyridiyl ring. For example, A can be pyridyl in which W is attached to the 2-position of the pyridyl ring, and R⁹ is attached to the 4- or the 6-position of the pyridyl ring. Such rings can be further substituted with 1, 2 or 3 R^{g} (e.g., halo, e.g., chloro; or NR^{g}R^{h}, e.g., NH₂).

Variable R⁹

R⁹ can be:

(9-i)—W²—S(O)ₙR¹⁰ or —W²—S(O)ₙNR¹¹R¹²; or

(9-ii) —W²—C(O)OR¹³; or

(9-iii) —W²—C(O)NR¹¹R¹²; or

(9-iv) C₁-C₁₂ alkyl or C₁-C₁₂ haloalkyl, each of which is:
(a) substituted with from 1 R^{h}, and
(b) optionally further substituted with from 1-5 R^{a};

or

(9-v)—NR¹⁴R¹⁵.

In some embodiments, R⁹ can be:
(9-i′) —W²—S(O)ₙR¹⁰; or
(9-ii), (9-iii), (9-iv), or (9-v).

In some embodiments, R⁹ can be any one of: (9-i), (9-i′), (9-ii), (9-iii), (9-iv), or (9-v). In certain embodiments, R⁹ can be —W²—S(O)ₙR¹⁰ or—W²—S(O)ₙNR¹¹R¹² (e.g., —W²—S(O)ₙR¹⁰. In other embodiments, R⁹ can be —W²—C(O)OR¹³.

In some embodiments, R⁹ can be any two of: (9-i), (9-i′), (9-ii), (9-iii), (9-iv), or (9-v). In certain embodiments, R⁹ can be —W²—S(O)ₙR¹⁰ or —W²—S(O)ₙNR¹¹R¹² (e.g., W²—S(O)ₙR¹⁰ and any one of (9-ii), (9-iii), (9-iv), or (9-v). For example, R⁹ can be:
—W²—S(O)ₙR¹⁰ or —W²—S(O)ₙNR¹¹R¹² (e.g., —W²—S(O)ₙR¹⁰); and
—W²—C(O)OR¹³.

In other embodiments, R⁹ can be any two of (9-ii), (9-iii), (9-iv), or (9-v).

In some embodiments, R⁹ can be any three of: (9-i), (9-i′), (9-ii), (9-iii), (9-iv), or (9-v).

In certain embodiments, R⁹ can be —W²—S(O)ₙR¹⁰, —W²—S(O)ₙNR¹¹R¹², and —W²—C(O)OR¹³.

In certain embodiments, R⁹ can be:
—W²—S(O)ₙR¹⁰ or —W²—S(O)ₙNR¹¹R¹² (e.g., —W²—S(O)ₙR¹⁰); and
—W²—C(O)OR¹³; and
any one of (9-iii), (9-iv), or (9-v).

In other embodiments, R⁹ can be (9-iii), (9-iv), or (9-v).

In some embodiments, R⁹ can be —W²—S(O)ₙR¹⁰ (e.g., —W²—S(O)₂R¹⁰, in which n is 2). In embodiments, W² can be a bond, i.e., R⁹ is connected to variable A by the sulfur (S) atom of the sulfinyl or the sulfonyl group.

In some embodiments, R¹⁰ can be C₁-C₆ (e.g., C₁-C₅) alkyl or C₁-C₆ (e.g., C₁-C₅ or C₁-C₃) haloalkyl, optionally substituted with from 1-2 R^{a}.

In certain embodiments, R¹⁰ can be C₂-C₆ alkyl, that is substituted with from 1-2 (e.g., 1) R^{a}.

In certain embodiments, R¹⁰ can be unsubstituted branched or unbranched C₁-C₆ (e.g., C₁-C₅, C₂-C₆, or C₃-C₆) alkyl. For example, R¹⁰ can be methyl (CH₃). As another example, R¹⁰ can be ethyl (CH₂CH₃). As a further example, R¹⁰ can be isopropyl (CH(CH₃)₂).

In certain embodiments, R¹⁰ can be branched or unbranched C₂-C₆ (e.g., C₃-C₆ or C₃-C₅) alkyl, which is substituted with 1 R^{a}. In embodiments, R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; halo; or heterocyclyl including 3-8 atoms, which is optionally substituted with from 1-5 R^{c}. In other embodiments, R^{a} can also be cyano. For example, R^{a} can be hydroxyl, C₁-C₆ (e.g., C₁-C₃) alkoxy, or NR^{m}Rⁿ. As another example, R^{a} can be cyano. In certain embodiments, R^{a} (e.g., hydroxyl) can be attached to a secondary or tertiary carbon atom of the alkyl group or a primary carbon of the alkyl group. In embodiments, R¹⁰ can be hydroxyl substituted C₃-C₆ (e.g., C₃-C₅) alkyl. In other embodiments, R¹⁰ can be C₃-C₆ (e.g., C₃-C₅) alkyl that is substituted with an amino group (NH₂) or a secondary or tertiary amino group.

In certain embodiments, R¹⁰ can be C₇-C₁₁ aralkyl (e.g., benzyl), optionally substituted with from 1-3 (e.g., 1-2, 1) R^{c}.

In certain embodiments, R¹⁰ can be C₆-C₁₀ aryl, optionally substituted with from 1-2 R^{d}.

In some embodiments, R⁹ can be —W²—S(O)ₙNR¹¹R¹² (e.g., —W²—S(O)₂NR¹¹R¹², in which n is 2). In embodiments, W² can be a bond, i.e., R⁹ is connected to variable A by the sulfur (S) atom of the sulfinamide or sulfonamide group.

In certain embodiments, one or both of R¹¹ and R¹² can be hydrogen. In certain embodiments, R⁹ can be —S(O)₂NH₂.

In other embodiments, one of R¹¹ and R¹² can be hydrogen, and the other of R¹¹ and R¹² can be:

(i) C₁-C₆ (e.g., C₁-C₃) alkyl or C₁-C₆ (e.g., C₁-C₃) haloalkyl, each of which is optionally substituted with from 1-5 (e.g., 1-4, 1-3, 1-2, 1) R^{a} (e.g., R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; or heterocyclyl including 3-8 atoms, which is optionally substituted with from 1-5 R^{c}); or

(iii) C₇-C₁₁ aralkyl, or heteroaralkyl including 6-11 atoms, each of which is optionally substituted with from 1-5 (e.g., 1-4, 1-3, 1-2, 1) R^{c}; or

(iv) C₆-C₁₀ aryl or heteroaryl including 5-10 atoms, each of which is optionally substituted with from 1-5 (e.g., 1-4, 1-3, 1-2, 1) R^{d}.

In certain embodiments, one of R¹¹ and R¹² can be hydrogen, and the other of R¹¹ and R¹² can be C₁-C₆ (e.g., C₁-C₃) alkyl or C₁-C₆ (e.g., C₁-C₃) haloalkyl, each of which is optionally substituted with from 1-5 (e.g., 1-4, 1-3, 1-2, 1) R^{a}. For example, one of R¹¹ and R¹² can be hydrogen, and the other of R¹¹ and R¹² can be C₁-C₆ (e.g., C₁-C₃) alkyl, such as CH₃.

In certain embodiments, R¹¹ and R¹² can each be, independently of one another:

(i) C₁-C₆ alkyl or C₁-C₆ haloalkyl, each of which is optionally substituted with from 1-5 R^{a}; or

(ii) C₂-C₆ alkenyl or C₂-C₆ alkynyl, each of which is optionally substituted with from 1-5 R^{b}; or

(iii) C₃-C₁₀ cycloalkyl, C₃-C₁₀ cycloalkenyl, heterocyclyl including 3-10 atoms, heterocycloalkenyl including 3-10 atoms, C₇-C₁₁ aralkyl, or heteroaralkyl including 6-11 atoms, each of which is optionally substituted with from 1-5 R^{c}; or

(iv) C₆-C₁₀ aryl or heteroaryl including 5-10 atoms, each of which is optionally substituted with from 1-5 R^{d}.

In certain embodiments, R¹¹ and R¹² can each be, independently of one another:

(i) C₁-C₆ (e.g., C₁-C₃) alkyl or C₁-C₆ (e.g., C₁-C₃) haloalkyl, each of which is optionally substituted with from 1-5 (e.g., 1-4, 1-3, 1-2, 1) R^{a} (e.g., R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; or heterocyclyl including 3-8 atoms, which is optionally substituted with from 1-5 R^{c}); or

(iii) C₇-C₁₁ aralkyl, or heteroaralkyl including 6-11 atoms, each of which is optionally substituted with from 1-5 (e.g., 1-4, 1-3, 1-2, 1) R^{c}; or

(iv) C₆-C₁₀ aryl or heteroaryl including 5-10 atoms, each of which is optionally substituted with from 1-5 (e.g., 1-4, 1-3, 1-2, 1) R^{d}.

In certain embodiments, R¹¹ and R¹² can each be, independently of one another:

(i) C₁-C₆ alkyl or C₁-C₆ haloalkyl, each of which is optionally substituted with from 1-5 R^{a}; or

C₇-C₁₁ aralkyl, or heteroaralkyl including 6-11 atoms, each of which is optionally substituted with from 1-5 R^{c}.

For example, R¹¹ and R¹² can each be, independently of one another C₁-C₆ alkyl or C₇-C₁₁ aralkyl, which is optionally substituted with from 1-5 (e.g., 1-4, 1-3, 1-2, 1) R^{c}.

In certain embodiments, R¹¹ and R¹² together with the nitrogen atom to which they are attached can form a heterocyclyl including 3-10 (e.g., 3-8, or 3-6) atoms or a heterocycloalkenyl including 3-10 (e.g., 3-8, or 3-6) atoms, each of which is optionally substituted with from 1-5 (1-4, 1-3, 1-2, 1) R^{c}. In some embodiments, the heterocyclyl can further include one or more additional ring heteroatoms (e.g., N, O, or S).

In certain embodiments, R¹¹ and R¹² together with the nitrogen atom to which they are attached can form a heterocyclyl including 3-10 (e.g., 3-8, 3-6, or 5-6) atoms, which is optionally substituted with from 1-5 (1-4, 1-3, 1-2, 1) R^{c}. For example, R¹¹ and R¹² together with the nitrogen atom to which they are attached can form a morpholinyl, piperidyl, pyrrolidinyl, or piperazinyl ring, each of which is optionally substituted with from 1-5 (1-4, 1-3, 1-2, 1) R^{c}.

In some embodiments, R⁹ can be —W²—C(O)OR¹³. In some embodiments, W² can be C₁-C₆ alkylene; or a bond. In certain embodiments, W² can be C₁-C₆ alkylene. For example, W² can be C₁-C₃ alkylene, such as CH₂ or CH₂CH₂. In other embodiments, W² can be a bond.

In some embodiments, R¹³ can be:

(i) hydrogen; or

(ii) C₁-C₆ (e.g., C₁-C₇) alkyl, which is optionally substituted with from 1-3 (e.g., 1-2, 1) R^{a}; or

(iii) C₃-C₇ cycloalkyl or C₇-C₁₁ aralkyl, each of which is optionally substituted with from 1-5 R^{c}; or

(iv) C₆-C₁₀ aryl or heteroaryl including 5-10 atoms, each of which is optionally substituted with from 1-5 R^{d}.

In certain embodiments, R¹³ can be hydrogen. In other embodiments, R¹³ can be a substituent other than hydrogen.

In some embodiments, R⁹ can be —W²—C(O)NR¹¹R¹².

Embodiments can include, for example, any one or more of the features described above in conjunction with —W²—S(O)ₙNR¹¹R¹² and/or —W²—C(O)OR¹³.

In some embodiments, R⁹ can be: C₁-C₆ alkyl or C₁-C₆ haloalkyl, each of which is (a) substituted with from 1 R^{h}, and (b) optionally further substituted with from 1 or 2 R^{a} (e.g., R^{a} can be C₃-C₇ cycloalkyl, which is optionally substituted with from 1-5 R^{c}); or

In certain embodiments, R^{h} at each occurrence can be, independently, hydroxyl, C₁-C₆ alkoxy, C₁-C₆ haloalkoxy; C₃-C₁₀ cycloalkoxy, which is optionally substituted with from 1-5 R^{c}; or C₆-C₁₀ aryloxy or heteroaryloxy including 5-10 atoms, each of which is optionally substituted with from 1-5 R^{d}.

In certain embodiments, R⁹ can have the following formula: —C(R⁹¹)(R⁹²)(R^{h}), in which each of R⁹¹ and R⁹² is, independently, C₁-C₁₂ alkyl or C₁-C₁₂ haloalkyl, each of which is optionally further substituted with from 1 or 2 R^{a} (e.g., R^{a} can be C₃-C₇ cycloalkyl, which is optionally substituted with from 1-5 R^{c}); C₃-C₇ cycloalkyl, which is optionally substituted with from 1-5 R^{c}; or C₆-C₁₀ aryl, which is optionally substituted with from 1-5 R^{d}; and R^{h} can be as defined anywhere herein.

In some embodiments, R⁹ can be —NR¹⁴R¹⁵, one of R¹⁴ and R¹⁵ is hydrogen or C₁-C₃ alkyl (e.g., hydrogen); and the other of R¹⁴ and R¹⁵ can be:

(i) —S(O)ₙR¹⁰; or

(ii) —C(O)OR¹³; or

(iii) —C(O)NR¹¹R¹²; or

(iv) C₁-C₁₂ alkyl or C₁-C₁₂ haloalkyl, each of which is:
(a) substituted with 1 R^{h}, and
(b) optionally further substituted with from 1-5 R^{a};

In embodiments, each of n, R¹⁰, R¹¹, R¹², R¹³, R^{h}, R^{a}, and R^{d} can be, independently, as defined anywhere herein. In embodiments, R¹³ can be other than hydrogen.

Variables R³, R⁴, and R⁵

In some embodiments, each of R³, R⁴, and R⁵ can be, independently:

(i) hydrogen; or

(ii) halo; or

(iii) C₁-C₆ alkyl or C₁-C₆ haloalkyl, each of which is optionally substituted with from 1-3 R^{a}; or

In certain embodiments, each of R³, R⁴, and R⁵ can be, independently:

(i) hydrogen; or

(ii) halo; or

(iii) C₁-C₃ alkyl or C₁-C₃ haloalkyl (e.g., perhaloalkyl, e.g., perfluoroalkyl), each of which is optionally substituted with from 1-3 R^{a}.

In certain embodiments, each of R³, R⁴, and R⁵ can be, independently, hydrogen or halo (e.g., fluoro).

In certain embodiments, each of R³, R⁴, and R⁵ can be hydrogen.

In certain embodiments, each of R³, R⁴, and R⁵ can be a substituent other than hydrogen (e.g., halo, e.g., fluoro).

In certain embodiments, one or two of R³, R⁴, and R⁵ can be hydrogen, and the other(s) can be:

(ii) halo; or

(iii) C₁-C₆ (e.g., C₁-C₃) alkyl or C₁-C₆ (e.g., C₁-C₃) haloalkyl (e.g., perhaloalkyl, e.g., perfluoroalkyl), each of which is optionally substituted with from 1-3 R^{a}.

In certain embodiments, when any of R³, R⁴, and R⁵ is alkyl that is substituted with one or more R^{a}, then the alkyl group is not directly substituted with an oxygen atom (e.g., R^{a} is other than hydroxyl, alkoxy, etc.).

Variable R⁶

In some embodiments, R⁶ can be:

(ii) halo; or

(iii) C₁-C₆ alkyl or C₁-C₆ haloalkyl, each of which is optionally substituted with from 1-3 R^{e}; or

(iv) cyano.

In some embodiments, R⁶ can be halo, cyano, C₁-C₆ (e.g., C₁-C₃) alkyl, or C₁-C₆ (e.g., C₁-C₃) haloalkyl.

In some embodiments, R⁶ can be chloro or bromo (e.g., chloro), cyano, C₁-C₆ (e.g., C₁-C₃) alkyl, or C₁-C₆ (e.g., C₁-C₃) haloalkyl.

In some embodiments, R⁶ can be halo, C₁-C₆ (e.g., C₁-C₃) alkyl, or C₁-C₆ (e.g., C₁-C₃) haloalkyl.

In some embodiments, R⁶ can be chloro or bromo (e.g., chloro), C₁-C₆ (e.g., C₁-C₃) alkyl, or C₁-C₆ (e.g., C₁-C₃) haloalkyl.

In some embodiments, R⁶ can be halo (e.g., chloro) or C₁-C₆ (e.g., C₁-C₃) haloalkyl (e.g., CF₃).

In some embodiments, R⁶ can be chloro or bromo (e.g., chloro) or C₁-C₆ (e.g., C₁-C₃) haloalkyl.

In certain embodiments, R⁶ can be chloro, cyano, CH₃, or CF₃. In certain embodiments, R⁶ can be chloro, CH₃, or CF₃. In certain embodiments, R⁶ can be chloro or CF₃.

In some embodiments, R⁶ can be hydrogen.

In some embodiments, R⁶ can be hydrogen, halo, cyano, C₁-C₆ (e.g., C₁-C₃) alkyl, or C₁-C₆ (e.g., C₁-C₃) haloalkyl.

In some embodiments, R⁶ can be hydrogen, chloro or bromo (e.g., chloro), cyano, C₁-C₆ (e.g., C₁-C₃) alkyl, or C₁-C₆ (e.g., C₁-C₃) haloalkyl.

In some embodiments, R⁶ can be hydrogen, halo, C₁-C₆ (e.g., C₁-C₃) alkyl, or C₁-C₆ (e.g., C₁-C₃) haloalkyl.

In some embodiments, R⁶ can be hydrogen, chloro or bromo (e.g., chloro), C₁-C₆ (e.g., C₁-C₃) alkyl, or C₁-C₆ (e.g., C₁-C₃) haloalkyl.

In some embodiments, R⁶ can be hydrogen, halo (e.g., chloro), or C₁-C₆ (e.g., C₁-C₃) haloalkyl (e.g., CF₃).

In some embodiments, R⁶ can be hydrogen, chloro or bromo (e.g., chloro), or C₁-C₆ (e.g., C₁-C₃) haloalkyl.

In certain embodiments, R⁶ can be hydrogen, chloro, cyano, CH₃, or CF₃. In certain embodiments, R⁶ can be hydrogen, chloro, CH₃, or CF₃. In certain embodiments, R⁶ can be hydrogen, chloro, or CF₃.

In some embodiments, R⁶ can be C₁-C₆ (e.g., C₁-C₃) haloalkyl (e.g., perfluoroalkyl, e.g., CF₃).

In some embodiments, R⁶ can be halo (e.g., chloro).

In some embodiments, R⁶ can be C₁-C₆ (e.g., C₁-C₃) alkyl (e.g., CH₃).

In some embodiments, R⁶ can be cyano.

In some embodiments, when R⁹ is —W²—S(O)ₙR¹⁰ or —W²—S(O)ₙNR¹¹R¹², then R⁶ can be hydrogen or hydrogen and any one or more of the permissible non-hydrogen substitutents delineated above for R⁶.

In some embodiments, when R⁹ is other than —W²—S(O)ₙR¹⁰ or —W²—S(O)ₙNR¹¹R¹², then R⁶ can be other than hydrogen.

A subset of compounds includes those in which:

R² can be C₆-C₁₀ aryl, which is (a) substituted with 1 R⁷; and (b) optionally substituted with from 1-4 (e.g., 1-2) R^{e}; and

A can be C₆-C₁₀ aryl, which is (a) substituted with from 1 R⁹; and (b) optionally substituted with from 1-4 R^{g}. In these embodiments, each of R⁷, R⁹, R^{e}, and R^{g} can be, independently, as defined anywhere herein.

In certain embodiments:

R² can be phenyl, which is (a) substituted with 1 R⁷ (i.e., WA); and (b) optionally substituted with from 1 R^{e}; and

A can be phenyl, which is (a) substituted with 1 R⁹; and (b) optionally substituted with from 1-4 R^{g}. In these embodiments, each of R⁷, R⁹, R^{e}, and R^{g} can be, independently, as defined anywhere herein.

A subset of compounds includes those in which R² has formula (C-1):

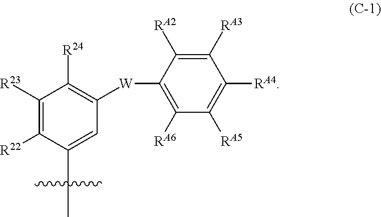

in which each of R²², R²³, and R²⁴ is, independently, hydrogen or R^{e}; and

one of R^{A2}, R^{A3}, R^{A4}, R^{A5}, and R^{A6} is R⁹, and the others are each, independently, hydrogen or R^{g}; and

W can be as defined anywhere herein.

In some embodiments:

(i) each of R²², R²³, and R²⁴ can be hydrogen; or

(ii) one of R²², R²³, and R²⁴ can be R^{e}, and the other two are hydrogen;

and

one of R^{A2}, R^{A3}, R^{A4}, R^{A5}, and R^{A6} can be R⁹, and the others are each, independently, hydrogen or R^{g}; and

W can be as defined anywhere herein.

Embodiments can include one or more of the following features.

W can be —O—, a bond, —OCH₂—, or —NH— (e.g., —O—, a bond, or —OCH₂—).

R^{e}, R⁹, and R^{g} can each be, independently, as defined anywhere herein.

Each of R²², R²³, and R²⁴ can be hydrogen; or each of R²², R²³, and R²⁴ can be a substituent other than hydrogen; or one or two of R²², R²³, and R²⁴ can be R^{e}, and the other(s) can be hydrogen.

One of R²², R²³, and R²⁴ can be R^{e}, and the other two can be hydrogen. For example, R²² can be R^{e}, and each of R²³ and R²⁴ can be hydrogen. In embodiments, R^{e} can be: halo (e.g., chloro); C₁-C₃ alkyl; or C₁-C₃ haloalkyl (e.g., C₁-C₃ fluoroalkyl, e.g., 1-5 fluorines can be present; or C₁-C₃ perfluoroalkyl). In certain embodiments, R^{e} can be halo (e.g., chloro).

One of R^{A3} and R^{A4} can be R⁹, the other of R^{A3} and R^{A4} can be hydrogen; and each of R^{A2}, R^{A5}, and R^{A6} can be, independently, hydrogen or R^{g}.

R^{A3} can be R⁹, R^{A4} can be hydrogen, and each of R^{A2}, R^{A5}, and R^{A6} can be hydrogen; or R^{A3} can be R⁹; R^{A4} can be hydrogen; one of R^{A2}, R^{A5}, and R^{A6} (e.g., R^{A5}) can be R^{g} (e.g., halo, e.g., fluoro) and the other two of R^{A2}, R^{A5}, and R^{A6} can be hydrogen.

R^{A4} can be R⁹, R^{A3} can be hydrogen, and each of R^{A2}, R^{A5}, and R^{A6} can be hydrogen. R^{A3} can be R⁹; R^{A4} can be hydrogen; one of R^{A2}, R^{A5}, and R^{A6} can be R^{g} (e.g., halo) and the other two of R^{A2}, R^{A5}, and R^{A6} can be hydrogen.

R⁹ can be —W²—S(O)ₙR¹⁰, in which n is 2, and each of W² and R¹⁰ can be as defined anywhere herein. For example, W² can be a bond. As another example, R¹⁰ can be C₁-C₆ alkyl, optionally substituted with from 1-2 R^{a}. In embodiments, R¹⁰ can be CH₃, CH₂CH₃, or isopropyl.

By way of example, R^{A3} can be —W²—S(O)ₙR¹⁰. n can be 2. W² can be a bond. R¹⁰ can be C₁-C₆ alkyl, optionally substituted with from 1-2 R^{a}. R¹⁰ can be C₁-C₃ alkyl (e.g., CH₃). R¹⁰ can be C₂-C₆ alkyl substituted with 1 R^{a} (e.g., R^{a} can be hydroxyl or C₁-C₃ alkoxy). Each of R^{A2}, R^{A4}, R^{A5}, and R^{A6} can be hydrogen. R^{A5} can be R^{g}, and each of R^{A2}, R^{A4}, and R^{A6} can be hydrogen.

R⁹ can be —W²—S(O)ₙNR¹¹R¹². W² can be a bond, and n can be 2. Each of R¹¹ and R¹² can be, independently, as defined anywhere herein. For example, each of R¹¹ and R¹² can be, independently: (i) hydrogen; or (ii) C₁-C₆ alkyl; or (iii) C₇-C₁₁ aralkyl, optionally substituted with from 1-5 R^{c}.

By way of example, R^{A3} can be —W²—S(O)ₙNR¹¹R¹². W² can be a bond; n can be 2, and each of R¹¹ and R¹² is, independently, as defined anywhere herein. For example, each of R¹¹ and R¹² can be, independently: (i) hydrogen; or (ii) C₁-C₆ alkyl; or (iii) C₇-C₁₁ aralkyl, optionally substituted with from 1-5 R^{c}. Each of R^{A2}, R^{A4}, R^{A5}, and R^{A6} can be hydrogen.

R⁹ can be —W²—C(O)OR¹³. Each of W² and R¹⁰ can be as defined anywhere herein. For example, W² can be a bond or C₁-C₆ alkylene. As another example, R¹³ can be hydrogen or C₁-C₆ alkyl.

By way of example, R^{A4} can be —W²—C(O)OR¹³. W² can be a bond or C₁-C₆ alkylene (e.g., CH₂). R¹³ can be hydrogen or C₁-C₃ alkyl. Each of R^{A2}, R^{A3}, R^{A5}, and R^{A6} can be hydrogen.

Other embodiments can include one of more other features described herein and present in combination with the features delineated above.

In some embodiments, the compounds can have formula (II):

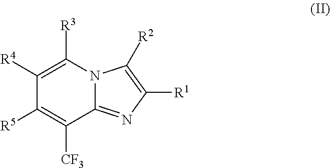

in which each of R¹, R², R³, R⁴, and R⁵ can be, independently, as defined anywhere herein (generically, subgenerically, or specifically).

In some embodiments, the compounds can have formula (III):

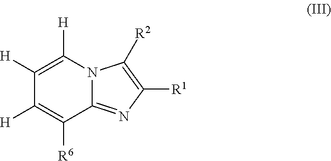

in which each of R¹, R², and R⁶ can be, independently, as defined anywhere herein (generically, subgenerically, or specifically).

In some embodiments, the compounds can have formula (IV):

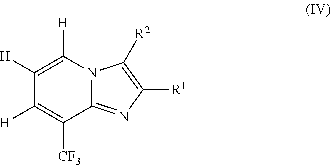

in which each of R¹ and R² can be, independently, as defined anywhere herein (generically, subgenerically, or specifically).

In some embodiments, the compounds can have formula (V):

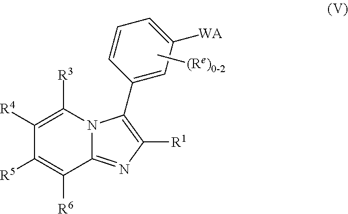

in which each of R¹, R³, R⁴, R⁵, R⁶, R^{e}, W, and A can be, independently, as defined anywhere herein (generically, subgenerically, or specifically).

In some embodiments, the compounds can have formula (VI):

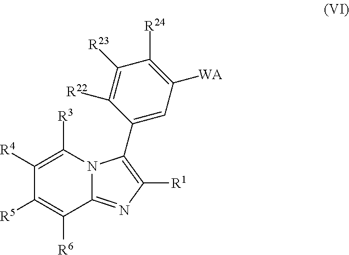

in which each of R¹, R³, R⁴, R⁵, R⁶, R²², R²³, R²⁴, W, and A can be, independently, as defined anywhere herein (generically, subgenerically, or specifically).

In some embodiments, the compounds can have formula (VII):

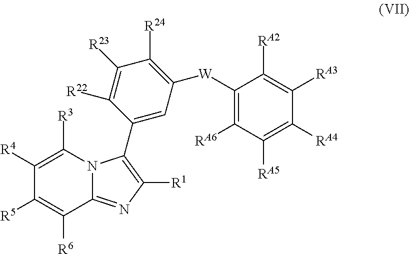

in which each of R¹, R³, R⁴, R⁵, R⁶, R²², R²³, R²⁴, R^{A2}, R^{A3}, R^{A4}, R^{A5}, R^{A6}, W, and A can be, independently, as defined anywhere herein (generically, subgenerically, or specifically).

In embodiments, the compounds of formulas (II), (III), (IV), (V), (VI), and (VII) can include any one or more of the following features.

R¹ can be:

(i) hydrogen; or

(ii) C₁-C₆ (e.g., C₁-C₃ or C₁-C₂) alkyl or C₁-C₆ (e.g., C₁-C₃ or C₁-C₂) haloalkyl; or

(iii) C₆-C₁₀ (e.g., phenyl) or heteroaryl including 5-10 (e.g., 5-6 atoms), each of which is optionally substituted with from 1-5 R^{d}; or

(iv) C₇-C₁₁ (e.g., C₇-C₁₀) aralkyl, or heteroaralkyl including 6-11 (e.g., 6-10) atoms, each of which is optionally substituted with from 1-5 (e.g., 1-4, 1-3, 1-2, 1) R^{c}.

R¹ can be hydrogen.

R¹ can be:

(ii) C₁-C₆ (e.g., C₁-C₃ or C₁-C₂) alkyl or C₁-C₆ (e.g., C₁-C₃ or C₁-C₂) haloalkyl; or

(iii) C₆-C₁₀ (e.g., phenyl), which is optionally substituted with from 1-5 R^{d}; or

(iv) C₇-C₁₁ (e.g., C₇-C₁₀) aralkyl, which is optionally substituted with from 1-5 (e.g., 1-4, 1-3, 1-2, 1) R^{c}.

R¹ can be:

(iii) heteroaryl including 5-10 (e.g., 5-6 atoms), which is optionally substituted with from 1-5 R^{d}; or

(iv) heteroaralkyl including 6-11 (e.g., 6-10) atoms, which is optionally substituted with from 1-5 (e.g., 1-4, 1-3, 1-2, 1) R^{c}.

R¹ can be: H; CH₃, CH₂CH₃, or CH(CH₃)₂; CF₃; phenyl, which is optionally substituted with from 1-5 R^{d}; or benzyl, which is optionally substituted with from 1-5 R^{c}.

R² can have formula (A), (A-1), (A-2), or (C-1) as defined anywhere herein.

W can be —O—.

W can be a bond.

W can be —W¹(C₁₋₆ alkylene)-. In certain embodiments, W¹ can be —O—. For example, W can be —O(C₁₋₃ alkylene)- (e.g., —OCH₂—).

W can be —(C₁₋₆ alkylene)W¹—. In certain embodiments, W¹ is —NR⁸—, in which R⁸ can be hydrogen; or W¹ can be —O—. In certain embodiments, W can be —(C₁₋₃ alkylene)NH— (e.g., —CH₂NH—). In certain embodiments, W can be —(C₁₋₃ alkylene)O— (e.g., —CH₂O—).

W can be —NR⁸—, (e.g., —NH—).

In some embodiments, A can be phenyl, which is (i) substituted with 1 R⁹ and (ii) optionally substituted with from 1-4 (e.g., 1-3, 1-2, 1) R^{g}, in which R^{g} can be as defined anywhere herein.

A can have formula (B-1). In embodiments, one of R^{A3} and R^{A4} is R⁹, and the other of R^{A3} and R^{A4} is hydrogen; and each of R^{A2}, R^{A5}, and R^{A6} is, independently, hydrogen or R^{g}, in which R⁹ and R^{g} can be as defined anywhere herein.

A can be heteroaryl including 5-10 atoms, which is (a) substituted with 1 R⁹; and (b) is optionally substituted with from 1-3 (e.g., 1-2, 1) R^{g}, in which R^{g} can be as defined anywhere herein.

Each of R^{e}, R⁹, and R^{g} can be, independently, as defined anywhere herein.

R⁹ can be:
—W²—S(O)ₙR¹⁰ or—W²—S(O)ₙNR¹¹R¹² (e.g., —W²—S(O)ₙR¹⁰); and/or
—W²—C(O)OR¹³.

Each of R¹⁰, R¹¹, R¹², and R¹³ can be, independently, as defined anywhere herein (e.g., as defined in conjunction with formula (C-1)).

W², n, R²², R²³, R²⁴, R^{A2}, R^{A3}, R^{A4}, R^{A5}, and R^{A6} can be as defined in conjunction with formula (C-1).

Each of R³, R⁴, and R⁵ can be hydrogen.

R⁶ can be:

(ii) halo; or

(iii) C₁-C₆ alkyl or C₁-C₆ haloalkyl, each of which is optionally substituted with from 1-3 R^{a}; or

(iv) cyano.

R⁶ can be halo (e.g., chloro) or C₁-C₆ (e.g., C₁-C₃) haloalkyl (e.g., CF₃).

One or more (e.g., 1, 2, or 3) of R¹, R³, R⁴, R⁵, and R⁶ (e.g., R¹ and/or R⁶) can be a substituent other than hydrogen.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

The compounds described herein can be synthesized according to methods described herein (or variations thereof) and/or conventional, organic chemical synthesis methods from commercially available starting materials and reagents or from starting materials and reagents that can be prepared according to conventional organic chemical synthesis methods. The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, compounds of formula (I) can be prepared according to Scheme 1.

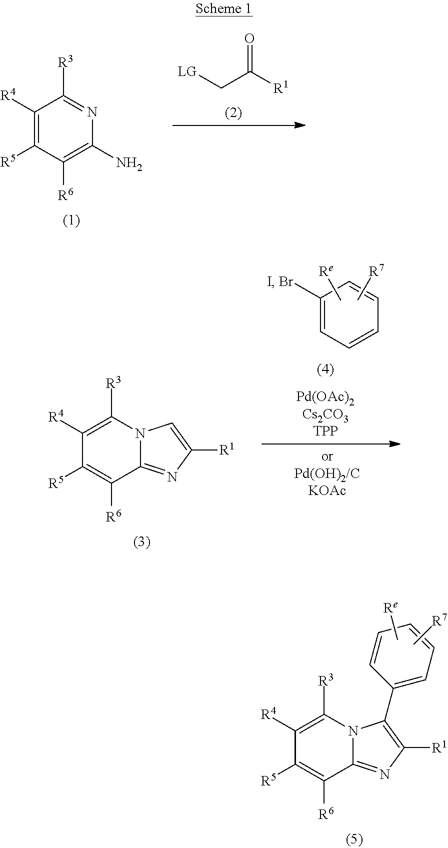

According to Scheme 1, the compounds of formula (I) can be prepared by reacting 2-aminopyridines (1) with alpha-halo-ketone (2), typically in the presence of a base (e.g., sodium bicarbonate) in a solvent such as ethanol at elevated temperatures, (e.g., 80-90° C.), for 16 to 24 h. Reaction of the resulting imidazo[1,2-a]pyridine (3) with a bromoarene or an iodoarene (4) in the presence of palladium catalyst (e.g., palladium(II)acetate), a base (e.g., cesium carbonate), and a ligand (e.g., triphenylphosphine) in a solvent such as 1,4-dioxane at elevated temperatures, (e.g., 100° C.), results in formation of compound (5). Alternatively, compounds (5) can be prepared by reaction of arene (4) in the presence of a palladium catalyst (e.g., 20% palladium hydroxide on carbon) and a base (e.g., potassium acetate) in a polar solvent (e.g., N,N-dimethylacetamide) at elevated temperatures, typically 145° C., for 12-18 h.

In some embodiments, the compounds of formula (I) can be prepared from intermediate compounds of formula (6). See Scheme 2 below. In these embodiments, Y in compound (4) is a halogen or protected hydroxyl group (e.g., methoxy or benzyloxy) and Z in compound (6) is a halogen or a hydroxyl group.

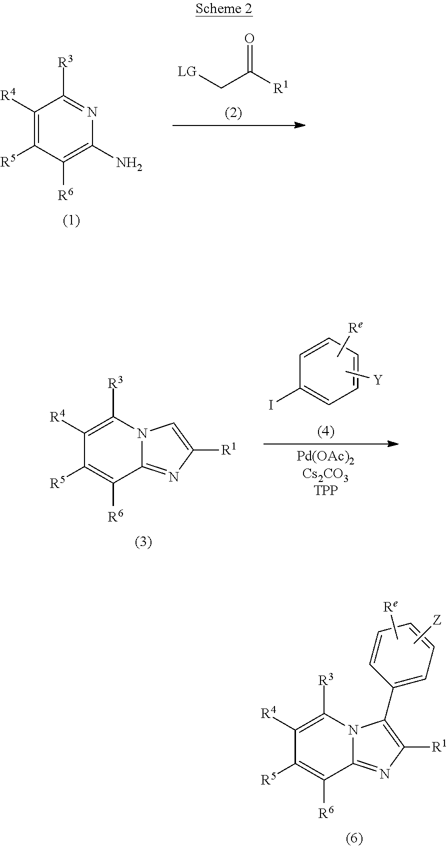

In certain embodiments, when Y in compound (4) is a protected hydroxyl group, the resultant O-protected coupling product can be deprotected according to conventional procedures to provide compounds of formula (6) in which Z is hydroxyl. As a non-limiting example, when Z is methoxy, deprotection can be carried out using pyridine hydrochloride at 200° C. for 0.5-2 h, or BBr₃ in dichloromethane.

In certain embodiments, compounds of formula (I) can be prepared according to Scheme 3.

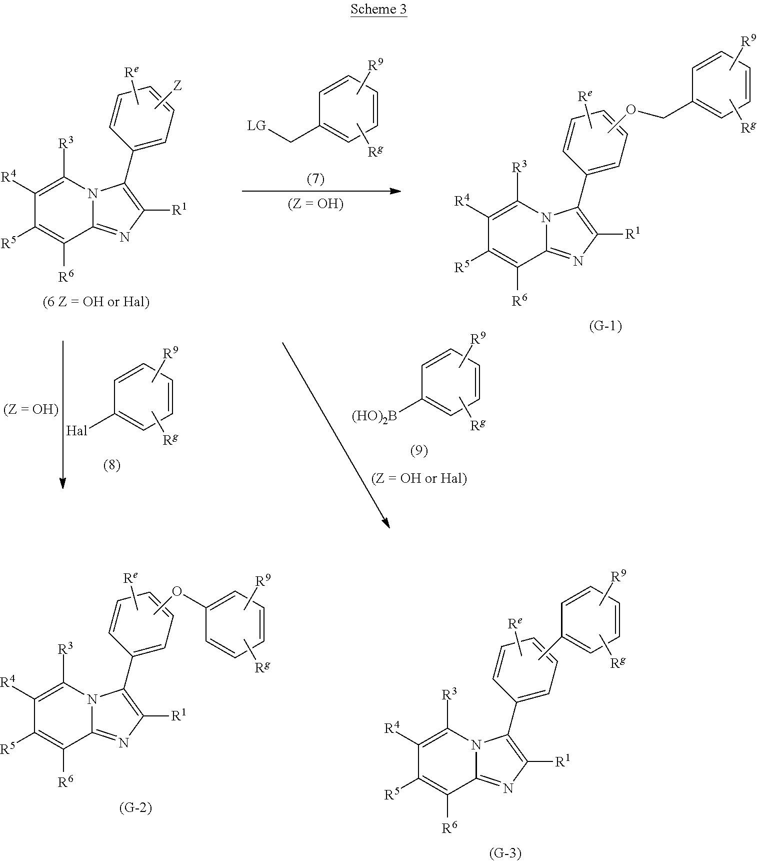

In embodiments, compounds of formula (6) in which Z is OH can be alkylated with an alkylating agent (7) and a suitable base to provide compounds of formula (G-1). The leaving group in compound (7) (i.e., LG) can be, e.g., a halogen or a sulfonate group (e.g., triflate). Suitable bases includes potassium carbonate, sodium carbonate, and cesium carbonate.

In other embodiments, compounds of formula (6) in which Z is OH can be treated with a halogenated compound (8) (wherein Hal in compound (8) is F, Cl, Br or I) to provide biarylether compounds of formula (G-2). In some embodiments, when the halogen is a fluorine or chlorine atom, biarylether formation can be accomplished using a polar solvent such as dimethylformamide or dimethylsulfoxide at elevated temperatures, (e.g., 100° C. to 150° C. for several hours by treatment) in the presence of a base, such as potassium carbonate. In other embodiments, when the halogen is a bromine or iodine, biarylether formation can be accomplished, e.g., via a metal-catalyzed coupling reaction using, e.g., a copper salt, such as CuI, or a palladium salt, in the presence of a base and a solvent, such as 1,4-dioxane, at elevated temperatures.

Still referring to Scheme 3, compounds of formula (G-3) can be prepared from compounds of formula (6), in which Z is OH or Hal (wherein Hal is Cl, Br or I). In some embodiments, when Z is OH, compounds of formula (6) are typically first converted into the corresponding sulfonate (e.g., triflate) under conventional sulfonating conditions, e.g., triflic anhydride and a tertiary amine such as triethylamine. Compounds of formula (6) in which Z is a sulfonate (e.g., OSO₂CF₃) or halide (e.g., Br or I) can then be coupled to an aryl boronic acid of formula (9), e.g., under Suzuki conditions to provide the compounds of formula (G-3).

In general, when R⁹ of the compounds of formula (G-1), (G-2) or (G-3) is or contains a carboxylic acid ester moiety, the ester can be converted to a carboxylic acid upon treatment with aqueous base (e.g., lithium hydroxide, sodium hydroxide or potassium hydroxide) in a suitable organic solvent. If the R⁹ group contains a haloalkyl group (e.g., CH₂X′ where X′ is a halogen, e.g., Br or Cl), then treatment with sodium cyanide in a suitable organic solvent can provide the corresponding cyanoalkyl compound (e.g., CH₂CN).

In some embodiments, compounds of formula (I) can be prepared according to Scheme 4.

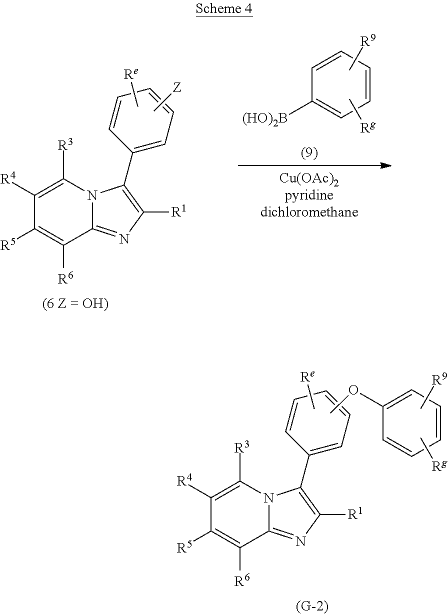

Referring to Scheme 4, a compound of formula (6, in which Z is OH, can be converted to biarylethers of formula (G-2), for example, by Cu(OAc)₂ mediated coupling of boronic acid (9) in the presence of base (e.g., pyridine) in a halogenated solvent (e.g., dichloromethane).

In some embodiments, compounds of formula (I) can be prepared according to Scheme 5.

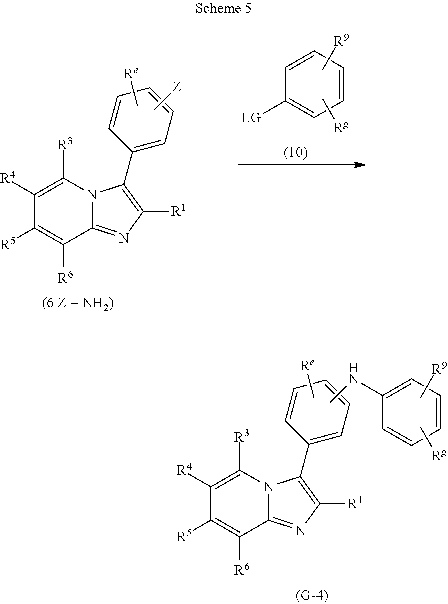

Referring to Scheme 5, compounds of formula (G-4) can be prepared from compounds (6), in which Z is NH₂, and compounds of formula (10), in which LG is a suitable leaving group (e.g., halide, triflate or boronic acid), under conventional coupling or displacement conditions.

In some embodiments, compounds of formula (I) can be prepared according to Scheme 6.

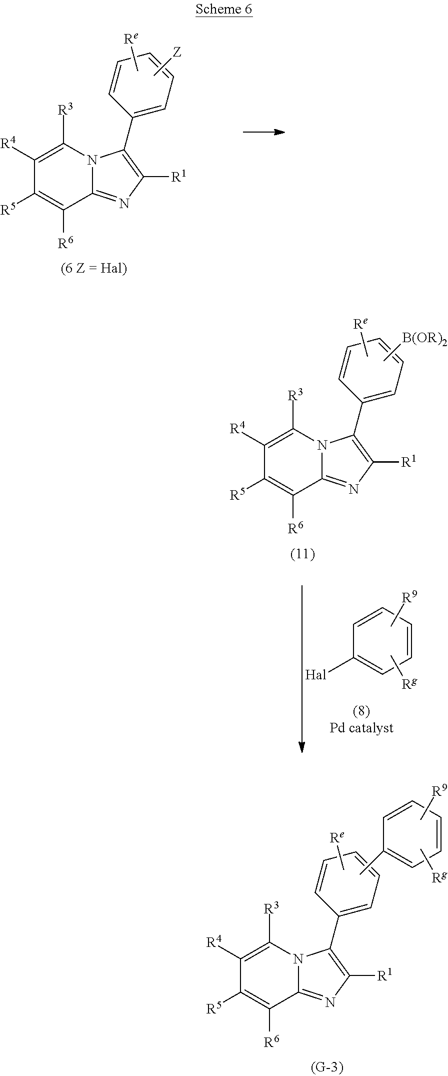

According to Scheme 6, compounds of formula (6), in which Z is a halogen (e.g., Br or I) can be converted to borolane (11) (e.g., R is H or alkyl) under conventional Suzuki conditions. Borolane (11) can then be coupled under conditions described above with aryl halide (8) (e.g., Hal is Br or I) to afford compounds of formula (G-3).

In some embodiments, compounds (7), (8), (9), or (10) can include a protected sulfonamide or amide moiety (i.e., R⁹ is —W²—SO₂NR¹¹R¹² or—W²—C(O)NR¹¹R¹², and one or both of R¹¹ and R¹² is a conventional protecting group). In some embodiments, R¹¹ can be an aralkyl group, e.g., 4-MeOPhCH₂—; and R¹² can be an aralkyl group, e.g., 4-MeOPhCH₂— or an alkyl group.

In some embodiments, when R⁹ in compounds (8) or (10) is an electron withdrawing group (e.g., wherein R⁹ is —W²—SO₂NR¹¹R¹² or—W²—C(O)NR¹¹R¹², and W² is a bond) and Hal or LG is a fluorine or chlorine atom located at a position that is para or ortho with respect to the R⁹ group, the fluorine or chlorine atom can typically be displaced by compounds bearing an amino or hydroxyl group in the presence of a base (see, e.g., Schemes 3 and 5). When Hal or LG in compounds (8) or (10) is a bromine, iodine, or sulfonate (e.g., triflate), such compounds can be coupled, e.g., to a boronic acid using a metal such as copper or palladium (see, e.g., Scheme 6).

Compounds of formula (I) in which R⁹ is a protected sulfonamide group (e.g., wherein R⁹ is —W²—SO₂N(4-MeOPhCH₂)R¹²) can be converted to the corresponding primary sulfonamide (R⁹ is —W²—SO₂NH₂ when R¹²=4-MeOPhCH₂) or secondary sulfonamide (X=SO₂NHR¹² when R¹² is other than 4-MeOPhCH₂), e.g., by treatment with trifluoroacetic acid in a halogenated solvent (e.g., dichloromethane) at ambient temperatures for several hours (e.g., 18 h). Compounds of formula (I) in which R⁹ is a protected amide (e.g., when R⁹ is —W²—C(O)N(4-MeOPhCH₂)R¹²) can be converted to the primary amides (R⁹ is —W²—C(O)NH₂ when R¹²=4-MeOPhCH₂) or secondary sulfonamide (R⁹ is —W²—C(O)NHR¹² when R¹² is other than 4-MeOPhCH₂) under similar conditions.

In some embodiments, compounds of formula (I) can be prepared according to Scheme 7.

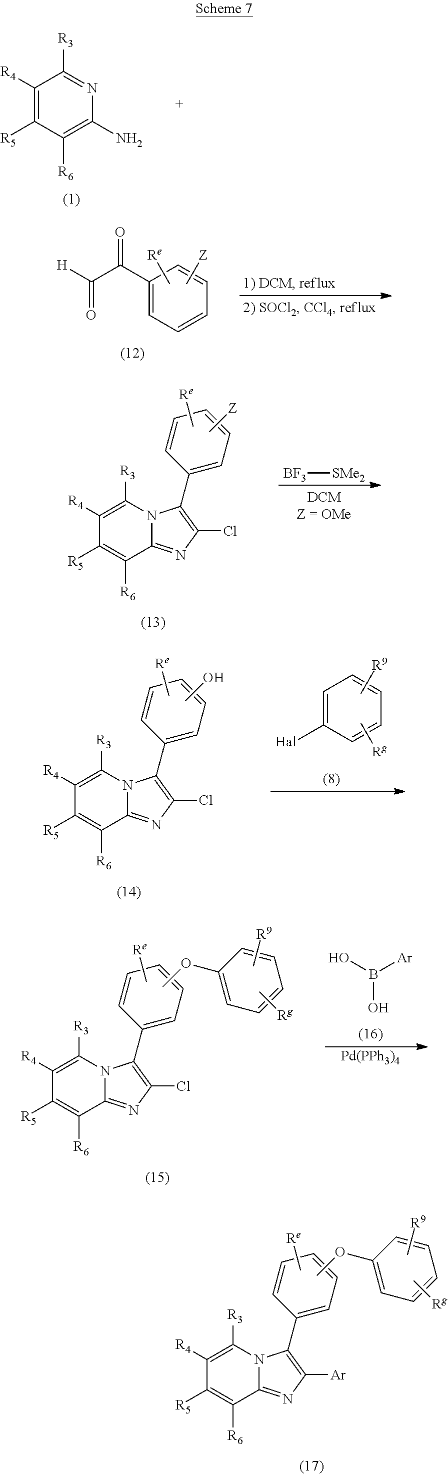

According to Scheme 7, compounds of formula (17) can be prepared by reacting 2-aminopyridines (1) with arylglyoxals (12) by refluxing in a solvent such as dichloromethane for 1-2 h, then removing the first solvent followed by heating at reflux in carbon tetrachloride in the presence of thionyl chloride for 0.5 h to provide (13). In certain embodiments when Z is methoxy, deprotection can be carried out using boron trifluoride-dimethylsulfide in dichloromethane to provide phenols (14). Conversion of phenol (14) into the biarylether can be accomplished as described in Scheme 3 to provide (15). Lastly, the chloro group can be reacted with aryl boronic acids (16) under Suzuki-type conditions to yield compounds of formula (17).

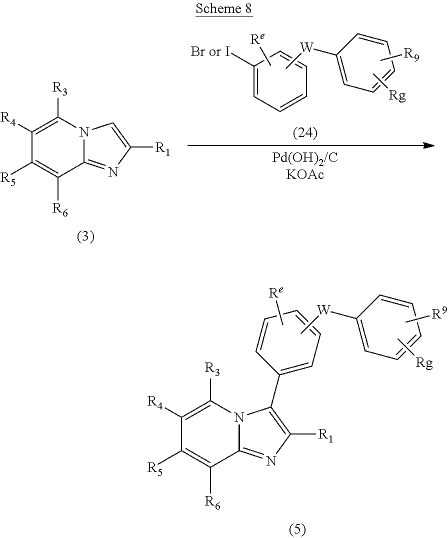

In some embodiments, the substituent corresponding to R² in formula (I) can be introduced by reacting compound (3) with the biaryl halide (24) to provide compound (5).

In some embodiments, compounds of formula (I) can be prepared according to Scheme 9.

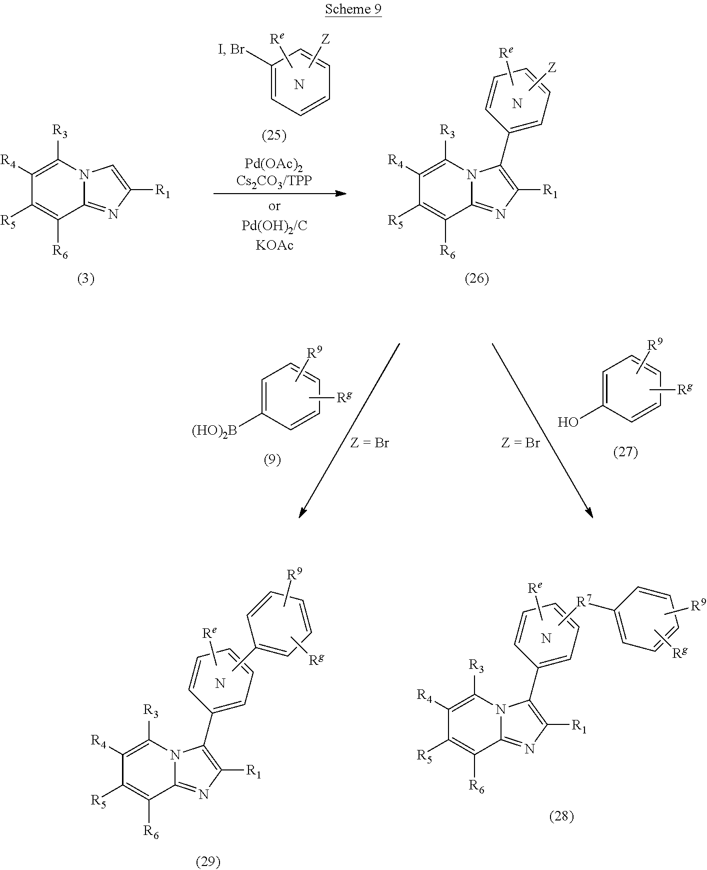

According to Scheme 9, compounds of formula (28) can be prepared by directly arylating imidazo[1,2-a]pyridines (3) with a halopyridine (25) to produce (26). Copper(0)-mediated coupling of compounds (26) in the presence of a base such as cesium carbonate in a polar solvent such as DMF can afford compound (28). Reaction of (26) with arylboronic acid (9) under Suzuki-type coupling conditions can provide compound (29).

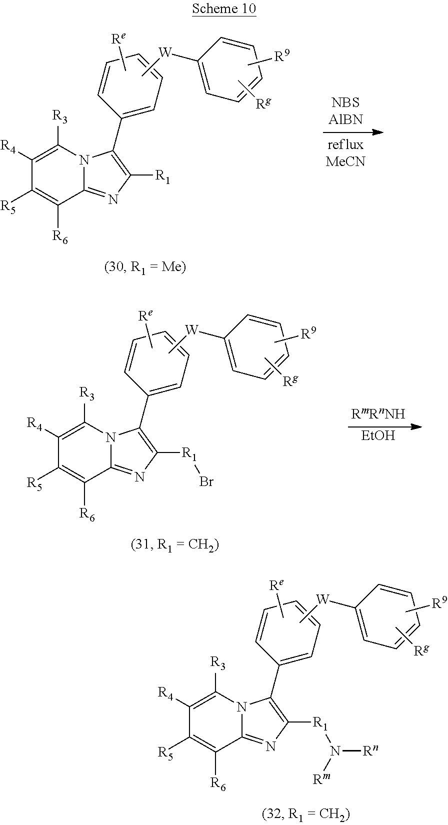

According to Scheme 10, compounds (32) can be synthesized via reacting 2-methylimidazo[1,2-a]pyridines (30) with a halogenating agent such as N-bromosuccinimide (NBS) with an initiator such as 2,2′-azobisisobutyronitrile (AIBN), in a polar solvent such as acetonitrile, at elevated temperatures, typically 85° C., for several hours to produce 2-(bromomethyl)imidazo[1,2-a]pyridines (31). Compounds (31) can then be reacted with amines in a polar solvent such as EtOH at ambient temperature to produce compounds (32).

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In general, the compounds described herein can be used for treating (e.g., controlling, ameliorating, alleviating, slowing the progression of, delaying the onset of, or reducing the risk of developing) or preventing one or more diseases, disorders, conditions or symptoms mediated by LXRs (e.g., cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A disorder or physiological condition that is mediated by LXR refers to a disorder or condition wherein LXR can trigger the onset of the condition, or where inhibition of a particular LXR can affect signaling in such a way so as to treat, control, ameliorate, alleviate, prevent, delay the onset of, slow the progression of, or reduce the risk of developing the disorder or condition. Examples of such disorders include, but are not limited to cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

While not wishing to be bound by theory, it is believed that LXR modulators that activate cholesterol efflux (e.g., upregulate ABCA1), but do not substantially increase SREBP-1c expression and triglyceride synthesis in liver, can both reduce atherosclerotic risk and minimize the likelihood of concommitantly increasing serum and hepatic triglyceride levels. Candidate compounds having differential activity for regulating ABCA1 (ABCG1) vs. SREBP-1c can be can be evaluated using conventional pharmacological test procedures, which measure the affinity of a candidate compound to bind to LXR and to upregulate the gene ABCA1.

In some embodiments, LXR ligands can be identified initially in cell-free LXR beta and LXR alpha competition binding assays. LXR ligands can be further characterized by gene expression profiling for tissue selective gene regulation.

In some embodiments, the compounds described herein have agonist activity for ABCA1 transactivation but do not substantially affect (e.g., inhibit) SREBP-1c gene expression in differentiated THP-1 macrophages. Gene expression analysis in an antagonist mode can be used to further delineate differential regulation of ABCA1 and SREBP-1c gene expression. In certain embodiments, the compounds described herein preferentially antagonize SREBP-1c activation (a marker for genes involved in cholesterol and fatty acid homeostasis) but do not substantially affect (e.g., have relatively minimal or additive effects) on ABCA1 gene expression or genes known to enhance HDL biogenesis (based on a competition assay with known potent synthetic LXR agonists). Cell type or tissue specificity may be further evaluated in additional cell lines, intestinal, CaCo2 or liver, HepG2 and Huh-7 cells where ABCA1 activity is believed to influence net cholesterol absorption and reverse cholesterol transport. The test procedures performed, and results obtained therefrom are described in the Examples section.

In some embodiments, the compounds described herein have agonist activity for ABCA1 and antagonist activity for SREBP-1c (e.g., as determined by gene specific modulation in cell based assays). In certain embodiments, the compounds described herein (in the agonist mode) have at least about 20% efficacy for ABCA1 activation by LXR and do not substantially agonize SREBP-1c (at most about 25% efficacy relative to a reference compound N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838)). In certain embodiments, the compounds described herein (in the antagonist mode) do not substantially antagonize ABCA1 gene expression. While not wishing to be bound by theory, it is believed that there may be an additive effect on ABCA1 gene expression relative to the reference compound at their EC₅₀ concentration. In certain embodiments, the compounds described herein (in the antagonist mode) inhibited agonist-mediated SREBP-1c gene expression in a dose dependent fashion.

In some embodiments, to study the effect of the compounds of formula (I) on skin aging, for example, in a clinical trial, cells can be isolated and RNA prepared and analyzed for the levels of expression of TIMP1, ABCA12, decorin, TNFα, MMP1, MMP3, and/or IL-8. The levels of gene expression (i.e., a gene expression pattern) can be quantified, for example, by Northern blot analysis or RT-PCR, by measuring the amount of protein produced, or by measuring the levels of activity of TIMP1, ABCA12, decorin, TNFα, MMP1, MMP3, and/or IL-8, all by methods known to those of ordinary skill in the art. In this way, the gene expression pattern can serve as a marker, indicative of the physiological response of the cells to the compounds of formula (I). Accordingly, this response state may be determined before, and at various points during, treatment of the individual with the compounds of formula (I).

In one embodiment, expression levels of cytokines and metalloproteases described herein can be used to facilitate design and/or identification of compounds that treat skin aging through an LXR-based mechanism. Accordingly, the invention provides methods (also referred to herein as “screening assays”) for identifying modulators, i.e., LXR modulators, that have a stimulatory or inhibitory effect on, for example, TIMP1, ABCA12, decorin, TNFα, MMP1, MMP3, and/or IL-8 expression.

An exemplary screening assay is a cell-based assay in which a cell that expresses LXR is contacted with a test compound, and the ability of the test compound to modulate TIMP1, ABCA12, decorin, TNFα, MMP1, MMP3, and/or IL-8 expression through an LXR-based mechanism. Determining the ability of the test compound to modulate TIMP1, ABCA12, decorin, TNFα, MMP1, MMP3, and/or IL-8 expression can be accomplished by monitoring, for example, DNA, mRNA, or protein levels, or by measuring the levels of activity of TIMP1, ABCA12, decorin, TNFα, MMP1, MMP3, and/or IL-8, all by methods known to those of ordinary skill in the art. The cell, for example, can be of mammalian origin, e.g., human.

In some embodiments, to study the effect of the compounds of formula (I) on osteoarthritis, for example, in a clinical trial, cells can be isolated and RNA prepared and analyzed for the levels of expression of ApoD and other genes implicated in osteoarthritis (for example, TNFα). The levels of gene expression (i.e., a gene expression pattern) can be quantified by Northern blot analysis or RT-PCR, by measuring the amount of protein produced, or by measuring the levels of activity of ApoD or other genes, all by methods known to those of ordinary skill in the art. In this way, the gene expression pattern can serve as a marker, indicative of the physiological response of the cells to the LXR modulator. Accordingly, this response state may be determined before, and at various points during, treatment of the individual with the LXR modulator.

An exemplary screening assay is a cell-based assay in which a cell that expresses LXR is contacted with a test compound, and the ability of the test compound to modulate ApoD expression and/or aggrecanase activity and/or cytokine elaboration through an LXR-based mechanism. Determining the ability of the test compound to modulate ApoD expression and/or aggrecanase activity and/or cytokine elaboration can be accomplished by monitoring, for example, DNA, mRNA, or protein levels, or by measuring the levels of activity of ApoD, aggrecanase, and/or TNFα, all by methods known to those of ordinary skill in the art. The cell, for example, can be of mammalian origin, e.g., human.

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of formula (I) (including any subgenera or specific compounds thereof)). In other embodiments, these agents may be part of a single dosage form, mixed together with the compounds of this invention in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of this invention include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/Kg to about 1000 mg/Kg, (e.g., from about 0.01 to about 100 mg/kg, from about 0.1 to about 100 mg/Kg, from about 1 to about 100 mg/Kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Remington: The Science and Practice of Pharmacy by University of the Sciences in Philadelphia (Editor); Publisher: Lippincott Williams & Wilkins; Twenty first Edition (May 1, 2005), which is incorporated herein by reference in its entirety.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The invention will be further described in the following examples. It should be understood that these examples are for illustrative purposes only and are not to be construed as limiting this invention in any manner.

### EXAMPLES

The following describes the preparation of representative compounds of this invention. Compounds described as homogeneous are determined to be of 90% or greater purity (exclusive of enantiomers) by analytical reverse phase chromatographic analysis with 254 nM UV detection. Melting points are reported as uncorrected in degrees centigrade. Mass spectral data is reported as the mass-to-charge ratio, m/z; and for high resolution mass spectral data, the calculated and experimentally found masses, [M+H]⁺, for the neutral formulae M are reported. All reactions are stirred and run under a nitrogen atmosphere unless otherwise noted. Eluents for chromatography are indicated by E for ethyl acetate and H for hexanes. Thus, for example, the expression “30:70 E:H” refers to a mixture of 30% ethyl acetate and 70% hexanes by volume.

### Example 1

### 2-methyl-8-(trifluoromethyl)imidazo[1,2-a]pyridine

A mixture of 3-(trifluoromethyl)pyridin-2-amine (1.00 g, 6.17 mmol), 1-chloropropan-2-one (0.685 g, 7.40 mmol), sodium bicarbonate (1.036 g, 12.34 mmol) and sodium iodide (0.277 g, 1.85 mmol) in ethanol (15 mL) was heated at reflux overnight. The cooled reaction mixture was partitioned between ethyl acetate and water. The layers were separated and the organic layer was washed with water and brine, dried over MgSO₄ and concentrated in vacuo. The resulting material was chromatographed using a 0:100 to 50:50 E:H gradient to afford the title compound as a yellow solid (0.625 g, 51%). MS (ES) m/z 201.

### Example 2

### 2-ethyl-8-(trifluoromethyl)imidazo[1,2-a]pyridine

A mixture of 3-(trifluoromethyl)pyridin-2-amine (3.00 g, 18.5 mmol), 1-bromobutan-2-one (3.76 g, 24.9 mmol), sodium hydrogencarbonate (3.11 g, 37.0 mmol) and sodium iodide (0.832 g, 5.55 mmol) in ethanol (37 mL) was refluxed overnight. The ethanol was removed in vacuo and the resulting material was treated with water and extracted with ethyl acetate. The combined organics were dried over MgSO₄ and concentrated. The product was chromatographed using a 0:100 to 50:50 E:H gradient to afford the title compound as a white solid (2.92, 74%). MS (ES) m/z 215.1

### Example 3

### 2-isopropyl-8-(trifluoromethyl)imidazo[1,2-a]pyridine

The title compound was prepared in a manner similar to that described in Example 2 except that 1-bromo-3-methylbutan-2-one was used in place of 1-bromo-2-butanone. MS (ES) m/z 229.

### Example 4

### 2-benzyl-8-(trifluoromethyl)imidazo[1,2-a]pyridine

The title compound was prepared in a manner similar to that described in Example 2 except that 1-bromo-3-phenylpropan-2-one was used in place of 1-bromo-2-butanone. MS (ES) m/z 277.

### Example 5

### 3-(2-methyl-8-(trifluoromethyl)imidazo[1,2-a]pyridin-3-yl)phenol

A mixture of 2-methyl-8-(trifluoromethyl)imidazo[1,2-a]pyridine (1.10 g, 5.50 mmol), 3-iodophenol (1.45 g, 6.59 mmol), cesium carbonate (1.79 g, 5.50 mmol), triphenylphosphine (0.058 g, 0.22 mmol) and diacetoxypalladium (0.025 g, 0.110 mmol) in dioxane (25 mL) was heated at reflux overnight. The cooled reaction was then treated with water, neutralized with dilute hydrochloric acid, and extracted with several portions of ethyl acetate. The combined organics were dried over MgSO₄ and concentrated onto Celite. The product was chromatographed using a 0:100 to 30:70 E:H gradient to afford the title compound as a tan solid (1.26 g, 78%). MS (ES) m/z 292.6; HRMS: calcd for C₁₅H₁₁F₃N₂O+H+, 293.08962. found (ESI, [M+H]⁺), 293.0883.

### Example 6

### 3-(2-ethyl-8-(trifluoromethyl)imidazo[1,2-a]pyridin-3-yl)phenol

The title compound was prepared in a manner similar to that described in Example 5 except that 2-ethyl-8-(trifluoromethyl)imidazo[1,2-a]pyridine was used in place of 2-methyl-8-(trifluoromethyl)imidazo[1,2-a]pyridine. MS (ES) m/z 307.

### Example 7

### 3-(2-isopropyl-8-(trifluoromethyl)imidazo[1,2-a]pyridin-3-yl)phenol

The title compound was prepared in a manner similar to that described in Example 5 except that 2-isopropyl-8-(trifluoromethyl)imidazo[1,2-a]pyridine was used in place of 2-methyl-8-(trifluoromethyl)imidazo[1,2-a]pyridine. MS (ES) m/z 320.

### Example 8

### 3-(2-benzyl-8-(trifluoromethyl)imidazo[1,2-a]pyridin-3-yl)phenol

The title compound was prepared in a manner similar to that described in Example 5 except that 2-benzyl-8-(trifluoromethyl)imidazo[1,2-a]pyridine was used in place of 2-methyl-8-(trifluoromethyl)imidazo[1,2-a]pyridine. MS (ES) m/z 369.

### Example 9

### 2-methyl-3-(3-(3-(methylsulfonyl)phenoxy)phenyl)-8-(trifluoromethyl)imidazo[1,2-a]pyridine

A mixture of 3-(2-methyl-8-(trifluoromethyl)imidazo[1,2-a]pyridin-3-yl)phenol (0.100 g, 0.342 mmol), 1-fluoro-3-(methylsulfonyl)benzene (0.119 g, 0.684 mmol) and potassium carbonate (0.095 g, 0.684 mmol) in DMF (3 mL) was heated at 150° C. overnight. The reaction mixture was cooled, diluted with water and extracted with several portions of ethyl acetate. The combined organics were washed with half-saturated brine and dried over MgSO₄. The extracts were concentrated in vacuo and chromatographed using a 0:100 to 25:75 E:H gradient to afford the title compound as a white solid (0.096 g, 63%). MS (ES) m/z 447.2; HRMS: calcd for C₂₂H₁₇F₃N₂O₃S+H+, 447.09847. found (ESI, [M+H]⁺), 447.0981.

### Example 10

### 2-methyl-3-(3-(3-(ethylsulfonyl)phenoxy)phenyl)-8-(trifluoromethyl)imidazo[1,2-a]pyridine

The title compound was prepared in a manner similar to that described in Example 9 except that 1-fluoro-3-(ethylsulfonyl)benzene was used in place of 1-fluoro-3-(methylsulfonyl)benzene. MS (ES) m/z 460.9; HRMS: calcd for C₂₃H₁₉F₃N₂O₃S+H+, 461.11412. found (ESI, [M+H]⁺), 461.1146.

### Example 11

### 2-methyl-3-(3-(3-(propylsulfonyl)phenoxy)phenyl)-8-(trifluoromethyl)imidazo[1,2-a]pyridine

The title compound was prepared in a manner similar to that described in Example 9 except that 1-fluoro-3-(propylsulfonyl)benzene was used in place of 1-fluoro-3-(methylsulfonyl)benzene. MS (ES) m/z 475.2; HRMS: calcd for C₂₄H₂₁F₃N₂O₃S+H+, 475.12977. found (ESI, [M+H]⁺), 475.1299.

### Example 12

### 2-methyl-3-(3-(3-(isopropylsulfonyl)phenoxy)phenyl)-8-(trifluoromethyl)imidazo[1,2-a]pyridine

The title compound was prepared in a manner similar to that described in Example 9 except that 1-fluoro-3-(isopropylsulfonyl)benzene was used in place of 1-fluoro-3-(methylsulfonyl)benzene. MS (ES) m/z 474.9; HRMS: calcd for C₂₄H₂₁F₃N₂O₃S+H+, 475.12977. found (ESI, [M+H]⁺), 475.1289.

### Example 13

### 3-[(3-{3-[2-methyl-8-(trifluoromethyl)imidazo[1,2-a]pyridin-3-yl]phenoxy}phenyl)sulfonyl]propan-1-ol

The title compound was prepared in a manner similar to that described in Example 9 except that 3-(3-fluorophenylsulfonyl)propan-1-ol was used in place of 1-fluoro-3-(methylsulfonyl)benzene. MS (ES) m/z 491.0; HRMS: calcd for C₂₄H₂₁F₃N₂O₄S+H+, 491.12469. found (ESI, [M+H]⁺), 491.1235.

### Example 14

### 2-methyl-4-[(3-{3-[2-methyl-8-(trifluoromethyl)imidazo[1,2-a]pyridin-3-yl]phenoxy}phenyl)sulfonyl]butan-2-ol

The title compound was prepared in a manner similar to that described in Example 9 except that 4-(3-fluorophenylsulfonyl)-2-methylbutan-2-ol was used in place of 1-fluoro-3-(methylsulfonyl)benzene. MS (ES) m/z 519.1; HRMS: calcd for C₂₆H₂₅F₃N₂O₄S+H+, 519.15599. found (ESI, [M+H]⁺), 519.1552.

### Example 15

### 3-{3-[3-fluoro-5-(methylsulfonyl)phenoxy]phenyl}-2-methyl-8-(trifluoromethyl)imidazo[1,2-a]pyridine

The title compound was prepared in a manner similar to that described in Example 9 except using 1,3-difluoro-5-(methylsulfonyl)benzene in place of 1-fluoro-3-(methylsulfonyl)benzene. MS (ES) m/z 465.1; HRMS: calcd for C₂₂H₁₆F₄N₂O₃S+H+, 465.08905. found (ESI, [M+H]⁺), 465.0902.

### Example 16

### 3-{3-[4-(sec-butylsulfonyl)phenoxy]phenyl}-2-methyl-8-(trifluoromethyl)imidazo[1,2-a]pyridine

The title compound was prepared in a manner similar to that described in Example 9 except that 1-fluoro-4-(sec-butylsulfonyl)benzene was used in place of 1-fluoro-3-(methylsulfonyl)benzene. MS (ES) m/z 489.2; HRMS: calcd for C₂₅H₂₃F₃N₂O₃S+H+, 489.14542. found (ESI, [M+H]⁺), 489.1474.

### Example 17

### 2-ethyl-3-(3-(3-(methylsulfonyl)phenoxy)phenyl)-8-(trifluoromethyl)imidazo[1,2-a]pyridine

A mixture of 3-(2-ethyl-8-(trifluoromethyl)imidazo[1,2-a]pyridin-3-yl)phenol (0.100 g, 0.326 mmol), 1-fluoro-3-(methylsulfonyl)benzene (0.114 g, 0.653 mmol) and potassium carbonate (0.090 g, 0.653 mmol) in DMF (3 mL) was heated at 150° C. overnight. The reaction mixture was cooled, diluted with water and extracted with several portions of ethyl acetate. The combined organics were washed with half-saturated brine and dried over MgSO₄. The extracts were concentrated onto Celite and chromatographed, typically using a 0:100 to 40:60 E:H gradient to afford the title compound as a white solid (0.163 g, 50%). HRMS: calcd for C₂₃H₁₉F₃N₂O₃S+H+, 461.11412. found (ESI, [M+H]⁺), 461.1149.

### Example 18

### 2-ethyl-3-(3-(3-(ethylsulfonyl)phenoxy)phenyl)-8-(trifluoromethyl)imidazo[1,2-a]pyridine

The title compound was prepared in a manner similar to that described in Example 17 except that 1-fluoro-3-(ethylsulfonyl)benzene was used in place of 1-fluoro-3-(methylsulfonyl)benzene. MS (ES) m/z 475.1; HRMS: calcd for C₂₄H₂₁F₃N₂O₃S+H+, 475.12977. found (ESI, [M+H]⁺), 475.1306.

### Example 19

### 2-ethyl-3-(3-(3-(propylsulfonyl)phenoxy)phenyl)-8-(trifluoromethyl)imidazo[1,2-a]pyridine

The title compound was prepared in a manner similar to that described in Example 17 except that 1-fluoro-3-(propylsulfonyl)benzene was used in place of 1-fluoro-3-(methylsulfonyl)benzene. HRMS: calcd for C₂₅H₂₃F₃N₂O₃S+H+, 489.14542. found (ESI, [M+H]⁺), 489.1459.

### Example 20

### 2-ethyl-3-(3-(3-(isopropylsulfonyl)phenoxy)phenyl)-8-(trifluoromethyl)imidazo[1,2-a]pyridine

The title compound was prepared in a manner similar to that described in Example 17 except that 1-fluoro-3-(isopropylsulfonyl)benzene was used in place of 1-fluoro-3-(methylsulfonyl)benzene. MS (ES) m/z 489.1; HRMS: calcd for C₂₅H₂₃F₃N₂O₃S+H+, 489.14542. found (ESI, [M+H]⁺), 489.1458.

### Example 21

### 3-(3-(3-(2-ethyl-84 trifluoromethyl)imidazo[1,2-a]pyridin-3-yl)phenoxy)phenylsulfonyl)propan-1-ol

The title compound was prepared in a manner similar to that described in Example 17 except that 3-(3-fluorophenylsulfonyl)propan-1-ol was used in place of 1-fluoro-3-(methylsulfonyl)benzene. MS (ES) m/z 505.1; HRMS: calcd for C₂₅H₂₃F₃N₂O₄S+H+, 505.14034. found (ESI, [M+H]⁺), 505.141.

### Example 22

### 4-(3-(3-(2-ethyl-8-(trifluoromethyl)imidazo[1,2-a]pyridin-3-yl)phenoxy)phenylsulfonyl)-2-methylbutan-2-ol

The title compound was prepared in a manner similar to that described in Example 17 except that 4-(3-fluorophenylsulfonyl)-2-methylbutan-2-ol was used in place of 1-fluoro-3-(methylsulfonyl)benzene. HRMS: calcd for C₂₇H₂₇F₃N₂O₄S+H+, 533.17164. found (ESI, [M+H]⁺), 533.1737.

### Example 23

### 5-(3-(3-(2-ethyl-8-(trifluoromethyl)imidazo[1,2-a]pyridin-3-yl)phenoxy)phenylsulfonyl)pentan-1-ol

The title compound was prepared in a manner similar to that described in Example 17 except that 5-(3-fluorophenylsulfonyl)pentan-1-ol was used in place of 1-fluoro-3-(methylsulfonyl)benzene. HRMS: calcd for C₂₇H₂₇F₃N₂O₄S+H+, 533.17164. found (ESI, [M+H]⁺), 533.1734.

### Example 24

### 2-ethyl-3-(3-(3-fluoro-5-(methylsulfonyl)phenoxy)phenyl)-8-(trifluoromethyl)imidazo[1,2-a]pyridine

The title compound was prepared in a manner similar to that described in Example 17 except that 1,3-difluoro-5-(methylsulfonyl)benzene was used in place of 1-fluoro-3-(methylsulfonyl)benzene. HRMS: calcd for C₂₃H₁₈F₄N₂O₃S+H+, 479.10470. found (ESI, [M+H]⁺), 479.1048.

### Example 25

### 2-isopropyl-3-(3-(3-(methylsulfonyl)phenoxy)phenyl)-8-(trifluoromethyl)imidazo[1,2-a]pyridine

A mixture of 3-(2-isopropyl-8-(trifluoromethyl)imidazo[1,2-a]pyridin-3-yl)phenol (0.064 g, 0.20 mmol), 1-bromo-3-(methylsulfonyl)benzene (0.094 g, 0.40 mmol), copper(I) iodide (3.8 mg, 0.020 mmol), cesium carbonate (0.195 g, 0.599 mmol) and N,N-dimethylglycine hydrochloride (10.5 mg, 0.075 mmol) in dioxane (3 mL) was heated at reflux overnight. The reaction was cooled, diluted with water and extracted with several portions of ethyl acetate. The combined organics were washed with half-saturated brine and dried over MgSO₄. The extracts were concentrated in vacuo and chromatographed using a 0:100 to 30:70 E:H gradient to afford the title compound as a white solid (0.060 g, 63%). MS (ES) m/z 474.9.

### Example 26

### 2-isopropyl-3-(3-(3-(ethylsulfonyl)phenoxy)phenyl)-8-(trifluoromethyl)imidazo[1,2-a]pyridine

The title compound was prepared in a manner similar to that described in Example 25 except that 1-bromo-3-(ethylsulfonyl)benzene was used in place of 1-bromo-3-(methylsulfonyl)benzene. MS (ES) m/z 489.0.

### Example 27

### 2-isopropyl-3-(3-(3-(isopropylsulfonyl)phenoxy)phenyl)-8-(trifluoromethyl)imidazo[1,2-a]pyridine

The title compound was prepared in a manner similar to that described in Example 25 except that 1-bromo-3-(isopropylsulfonyl)benzene was used in place of 1-bromo-3-(methylsulfonyl)benzene. MS (ES) m/z 502.9.

### Example 28

### 2-benzyl-3-(3-(3-(methylsulfonyl)phenoxy)phenyl)-8-(trifluoromethyl)imidazo[1,2-a]pyridine

A mixture of 3-(2-benzyl-8-(trifluoromethyl)imidazo[1,2-a]pyridin-3-yl)phenol (0.100 g, 0.27 mmol), 3-(methylsulfonyl)phenylboronic acid (0.109 g, 0.543 mmol), diacetoxycopper (0.049 g, 0.27 mmol), pyridine (0.066 mL, 0.81 mmol) and 4A molecular sieves (0.250 g) in dichloromethane (7 mL) was stirred open to the air overnight at room temperature. The reaction was filtered through Celite and concentrated in vacuo. The product was chromatographed using a 0:100 to 25:75 E:H gradient to afford impure compound. Therefore, further purification via reverse phase chromatography using a 0:100 to 100:0 acetonitrile:water gradient gave the title compound as a white solid (0.052 g, 37%). MS (ES) m/z 522.9; HRMS: calcd for C₂₈H₂₁F₃N₂O₃S+H+, 523.12977. found (ESI, [M+H]⁺), 523.1299.

### Example 29

### 2-benzyl-3-(3-(3-(ethylsulfonyl)phenoxy)phenyl)-8-(trifluoromethyl)imidazo[1,2-a]pyridine

The title compound was prepared in a manner similar to that described in Example 28 except that 3-(ethylsulfonyl)phenylboronic acid was used in place of 3-(methylsulfonyl)phenylboronic acid. MS (ES) m/z 536.9; HRMS: calcd for C₂₉H₂₃F₃N₂O₃S+H+, 537.14542. found (ESI, [M+H]⁺), 537.1454.

### Example 30

### 2-benzyl-3-(3-(3-(isopropylsulfonyl)phenoxy)phenyl)-8-(trifluoromethyl)imidazo[1,2-a]pyridine

A mixture of 3-(2-benzyl-8-(trifluoromethyl)imidazo[1,2-a]pyridin-3-yl)phenol (0.100 g, 0.271 mmol), 1-bromo-3-(isopropylsulfonyl)benzene (0.143 g, 0.54 mmol), copper(I) iodide (5.2 mg, 0.027 mmol), cesium carbonate (0.265 g, 0.814 mmol) and N,N-dimethylglycine hydrochloride (0.014 g, 0.10 mmol) in dioxane (3 mL) was heated at reflux overnight. The cooled reaction was diluted with water and extracted with several portions of ethyl acetate. The extracts were dried over MgSO4, concentrated in vacuo, and chromatographed using a 0:100 to 30:70 E:H gradient to afford the title compound as a white solid (0.035 g, 23%). MS (ES) m/z 550.9.

### Example 31

### 3-bromo-N-(4-methoxybenzyl)benzenesulfonamide

A stirred mixture of 3-bromobenzenesulfonyl chloride (5.11 g, 20.0 mmol) and triethylamine (3.07 mL, 22.0 mmol) in dichloromethane (100 mL) was treated with 4-methoxybenzylamine (2.85 mL, 22.0 mmol) over 5 min. After 2 h at ambient temperature, the reaction was treated with saturated aqueous NaHCO₃ (100 mL) and extracted with dichloromethane (2×50 mL). The combined extracts were dried (MgSO₄) and concentrated in vacuo to a solid. Chromatography eluting with a 30:70 to 50:50 E:H gradient afforded the title compound as a white solid (6.18 g, R_{f} ˜0.4 in 50:50 E:H).

### Example 32

### 3-bromo-N,N-bis(4-methoxybenzyl)benzenesulfonamide

A solution of 3-bromo-N-(4-methoxybenzyl)benzenesulfonamide (3.56 g, 10.0 mmol) in DMF (20 mL) was treated with 60% NaH in oil (440 mg, 11.0 mmol). Gas evolved. After 20 min, 4-methoxybenzyl chloride (1.63 mL, 12.0 mmol) was added and the reaction is stirred at ambient temperature for 5 h. The reaction was poured into water (60 mL) resulting in a white precipitate. The precipitate was filtered off, washed with water, and dried in vacuo to afford the title compound as a white solid (4.92 g, R_{f} ˜0.5 in E:H).

### Example 33

### 3-bromo-N-(4-methoxybenzyl)-N-methylbenzenesulfonamide

A solution of 3-bromo-N-(4-methoxybenzyl)benzenesulfonamide (2.49 g, 7.00 mmol) in DMF (20 mL) was treated with 60% NaH in oil (336 mg, 8.4 mmol). Gas evolved. After 10 min, methyl iodide (0.66 mL, 10.5 mmol) was added and the reaction is stirred at ambient temperature overnight. The reaction was poured into water (60 mL) and extracted with ethyl acetate (2×40 mL). The extracts are was dried (MgSO₄), concentrated in vacuo, and chromatographed using a 20:80 to 50:50 E:H gradient to afford the title compound as a white solid (2.21 g, R_{f} ˜0.5 in 50:50 E:H).

### Example 34

### 4-fluoro-N-(4-methoxybenzyl)benzenesulfonamide

The title compound was prepared in a manner similar to that described in Example 31 except that 4-fluorobenzenesulfonyl chloride was used in place of 3-bromobenzenesulfonyl chloride, isolating a white solid.

### Example 35

### 4-fluoro-N,N-bis(4-methoxybenzyl)benzenesulfonamide

A solution of 4-fluoro-N-(4-methoxybenzyl)benzenesulfonamide (2.95 g, 10.0 mmol) in DMF (20 mL) was treated with 60% NaH in oil (440 mg, 11.0 mmol). Gas evolved. After 20 min, 4-methoxybenzyl chloride (1.63 mL, 12.0 mmol) was added and the reaction was stirred at ambient temperature for 5 h. The reaction was treated with water (60 mL) producing a white precipitate. The material was chromatographed using a 20:80 to 40:60 E:H gradient to afford the title compound as a white solid (3.08 g).

### Example 36

### 3-(3-(8-chloro-2-isopropylimidazo[1,2-a]pyridin-3-yl)phenoxy)-N,N-bis(4-methoxybenzyl)benzenesulfonamide

A mixture of 3-(2-isopropyl-8-chloro-imidazo[1,2-a]pyridin-3-yl)phenol (143 mg, 0.50 mmol), 3-bromo-N,N-bis(4-methoxybenzyl)benzenesulfonamide (357 mg, 0.75 mmol), CuI (40 mg, 0.20 mmol), Me₂NCH₂CO₂H hydrochloride (53 mg, 0.375 mmol), and cesium carbonate (489 mg, 150 mmol) in 1,4-dioxane (8.0 mL) was heated at 105° C. under nitrogen. After 18 h, the reaction was treated with water (20 mL) and extracted with ethyl acetate (3×10 mL). The extracts were dried (MgSO₄), concentrated in vacuo, and chromatographed on silica gel using a 15:85 to 50:50 E:H gradient and then by reverse phase chromatography using a 0:100 to 100:0 acetonitrile:water gradient to afford the title compound as a white solid (215 mg, R_{f} ˜0.15 in 35:65 E:H).

### Example 37

### 4-(3-(8-chloro-2-isopropylimidazo[1,2-a]pyridin-3-yl)phenoxy)-N,N-bis(4-methoxybenzyl)benzenesulfonamide

A mixture of 3-(2-isopropyl-8-chloro-imidazo[1,2-a]pyridin-3-yl)phenol (143 mg, 0.50 mmol), 4-fluoro-N,N-bis(4-methoxybenzyl)benzenesulfonamide (249 mg, 0.60 mmol), and 138 mg (1.00 mmol) in DMF (5.0 mL) was heated at 130° C. under nitrogen for 18 h. The reaction was cooled, treated with water (20 mL), and extracted with ethyl acetate (3×10 mL). The dried (MgSO₄) extracts were concentrated in vacuo and chromatographed on silica gel using a 20:80 to 50:50 E:H gradient to afford the title compound as a white solid (326 mg, R_{f} ˜0.15 in 35:65 E:H).

### Example 38

### 3-(3-(8-chloro-2-isopropylimidazo[1,2-a]pyridin-3-yl)phenoxy)benzenesulfonamide

3-(3-(8-Chloro-2-isopropylimidazo[1,2-a]pyridin-3-yl)phenoxy)-N,N-bis(4-methoxybenzyl)benzenesulfonamide (182 mg, mmol) in dichloromethane (2.0 mL) was treated with trifluoroacetic acid (2.0 mL) and stirred at ambient temperature for 18 h, then concentrated in vacuo. The residue was treated with saturated aqueous NaHCO₃ (10 mL) and extracted with dichloromethane (3×10 mL). The extracts were dried (MgSO₄) and concentrated in vacuo. The residue was purified by chromatography eluting with an E:H gradient to afford the title compound as a white solid.

### Example 39

### 4-(3-(8-chloro-2-isopropylimidazo[1,2-a]pyridin-3-yl)phenoxy)benzenesulfonamide

The title compound was prepared in a manner similar to that described in Example 38 except that 4-(3-(8-chloro-2-isopropylimidazo[1,2-a]pyridin-3-yl)phenoxy)-N,N-bis(4-methoxybenzyl)benzenesulfonamide (279 mg) was used as the substrate.

### Example 40

### 3-{3-[2-ethyl-8-(trifluoromethyl)imidazo[1,2-a]pyridin-3-yl]phenoxy}-N-(4-methoxybenzyl)-N-methylbenzenesulfonamide

In essentially the same manner as Example 36 but using 3-(2-ethyl-8-(trifluoromethyl)imidazo[1,2-a]pyridin-3-yl)phenol and 3-bromo-N-(4-methoxybenzyl)-N-methylbenzenesulfonamide as starting materials was prepared the title compound as a colorless oil. MS (ES) m/z 595.8. HRMS: calcd for C₃₁H₂₈F₃N₃O₄S+H+, 596.18254. found (ESI, [M+H]+ Obs'd), 596.1827.

### Example 41

### 3-{3-[2-isopropyl-8-(trifluoromethyl)imidazo[1,2-a]pyridin-3-yl]phenoxy}-N,N-bis(4-methoxybenzyl)benzenesulfonamide

In essentially the same manner as Example 36 but using 3-(2-isopropyl-8-(trifluoromethyl)imidazo[1,2-a]pyridin-3-yl)phenol and 3-bromo-N,N-bis(4-methoxybenzyl)benzenesulfonamide as starting materials was prepared the title compound as a colorless oil. MS (ES) m/z 715.8. HRMS: calcd for C₃₉H₃₆F₃N₃O₅S+H+, 716.24005. found (ESI, [M+H]+ Obs'd), 716.2397.

### Example 42

### 3-[3-(8-chloro-2-ethylimidazo[1,2-a]pyridin-3-yl)phenoxy]-N,N-bis(4-methoxybenzyl)benzenesulfonamide

In essentially the same manner as Example 36 but using 3-(8-chloro-2-ethylimidazo[1,2-a]pyridin-3-yl)phenol and 3-bromo-N,N-bis(4-methoxybenzyl)benzenesulfonamide as starting materials was prepared the title compound as a pale yellow solid. MS (ES) m/z 667.7. HRMS: calcd for C₃₇H₃₄ClN₃O₅S+H+, 668.19805. found (ESI, [M+H]+ Obs'd), 668.1986.

### Example 43

### 3-[3-(8-chloro-2-ethylimidazo[1,2-a]pyridin-3-yl)phenoxy]-N-(4-methoxybenzyl)-N-methylbenzenesulfonamide

In essentially the same manner as Example 36 but using 3-(2-ethyl-8-chloroimidazo[1,2-a]pyridin-3-yl)phenol and 3-bromo-N-(4-methoxybenzyl)-N-methylbenzenesulfonamide as starting materials was prepared the title compound as an off-white solid. MS (ES) m/z 561.7; HRMS: calcd for C₃₀H₂₈ClN₃O₄S+H+, 562.15618. found (ESI, [M+H]+ Obs'd), 562.1559.

### Example 44

### 3-[3-(8-chloro-2-isopropylimidazo[1,2-a]pyridin-3-yl)phenoxy]-N-(4-methoxybenzyl)-N-methylbenzenesulfonamide

In essentially the same manner as Example 36 but using 3-(8-chloro-2-isopropylimidazo[1,2-a]pyridin-3-yl)phenol and 3-bromo-N-(4-methoxybenzyl)-N-methylbenzenesulfonamide as starting materials was prepared the title compound as a white solid from a foam. MS (ES) m/z 575.7. HRMS: calcd for C₃₁H₃₀ClN₃O₄S+H+, 576.17183. found (ESI, [M+H]+ Obs'd), 576.1720.

### Example 45

### 3-[3-(8-cyano-2-ethylimidazo[1,2-a]pyridin-3-yl)phenoxy]-N,N-bis(4-methoxybenzyl)benzenesulfonamide

In essentially the same manner as Example 36 but using 3-(8-cyano-2-ethylimidazo[1,2-a]pyridin-3-yl)phenol and 3-bromo-N,N-bis(4-methoxybenzyl)benzenesulfonamide as starting materials was prepared the title compound as a light yellow solid. MS (ESI) m/z 659. HRMS: calcd for C₃₈H₃₄N₄O₅S+H+, 659.23227. found (ESI, [M+H]+ Obs'd), 659.2323.

### Example 46

### 3-[3-(8-cyano-2-ethylimidazo[1,2-a]pyridin-3-yl)phenoxy]-N-(4-methoxybenzyl)-N-methylbenzenesulfonamide

In essentially the same manner as Example 36 but using 3-(8-cyano-2-ethylimidazo[1,2-a]pyridin-3-yl)phenol and 3-bromo-N-(4-methoxybenzyl)-N-methylbenzenesulfonamide as starting materials was prepared the title compound as a light yellow solid. MS (ES) m/z 552.7. HRMS: calcd for C₃₁H₂₈N₄O₄S+H+, 553.19040. found (ESI, [M+H]+ Obs'd), 553.1903.

### Example 47

### 3-[3-(8-chloro-2-ethylimidazo[1,2-a]pyridin-3-yl)phenoxy]benzenesulfonamide

In essentially the same manner as Example 38 but using 3-[3-(8-chloro-2-ethylimidazo[1,2-a]pyridin-3-yl)phenoxy]-N,N-bis(4-methoxybenzyl)benzenesulfonamide as starting material was prepared the title compound as a white solid. MS (ES) m/z 427.7; HRMS: calcd for C₂₁H₁₈ClN₃O₃S+H+, 428.08301. found (ESI, [M+H]+ Obs'd), 428.0835.

### Example 48

### 3-[3-(8-chloro-2-ethylimidazo[1,2-a]pyridin-3-yl)phenoxy]-N-methylbenzenesulfonamide

In essentially the same manner as Example 38 but using 3-[3-(8-chloro-2-ethylimidazo[1,2-a]pyridin-3-yl)phenoxy]-N-(4-methoxybenzyl)-N-methylbenzenesulfonamide as starting material was prepared the title compound as a white solid. MS (ES) m/z 441.6. HRMS: calcd for C₂₂H₂₀ClN₃O₃S+H+, 442.09866. found (ESI, [M+H]+ Obs'd), 442.0992.

### Example 49

### 3-[3-(8-chloro-2-isopropylimidazo[1,2-a]pyridin-3-yl)phenoxy]-N-methylbenzenesulfonamide

In essentially the same manner as Example 38 but using 3-[3-(8-chloro-2-isopropylimidazo[1,2-a]pyridin-3-yl)phenoxy]-N-(4-methoxybenzyl)-N-methylbenzenesulfonamide as starting material was prepared the title compound as an off-white solid. MS (ES) m/z 455.6; HRMS: calcd for C₂₃H₂₂ClN₃O₃S+H+, 456.11431. found (ESI, [M+H]+ Obs'd), 456.1145.

### Example 50

### 3-[3-(8-cyano-2-ethylimidazo[1,2-a]pyridin-3-yl)phenoxy]benzenesulfonamide

In essentially the same manner as Example 38 but using 3-[3-(8-cyano-2-ethylimidazo[1,2-a]pyridin-3-yl)phenoxy]-N,N-bis(4-methoxybenzyl)benzenesulfonamide as starting material was prepared the title compound as a pale yellow solid. MS (ES) m/z 418.7. HRMS: calcd for C₂₂H₁₈N₄O₃S+H+, 419.11724. found (ESI, [M+H]+ Obs'd), 419.1174.

### Example 51

### 3-[3-(8-cyano-2-ethylimidazo[1,2-a]pyridin-3-yl)phenoxy]-N-methylbenzenesulfonamide

In essentially the same manner as Example 38 but using 3-[3-(8-cyano-2-ethylimidazo[1,2-a]pyridin-3-yl)phenoxy]-N-(4-methoxybenzyl)-N-methylbenzenesulfonamide as starting material was prepared the title compound as a light yellow solid. MS (ES) m/z 432.7. HRMS: calcd for C₂₃H₂₀N₄O₃S+H+, 433.13289. found (ESI, [M+H]+ Obs'd), 433.1335.

### Example 52

### 2-isopropyl-3-(3-{[3-(methylsulfonyl)benzyl]oxy}phenyl)-8-(trifluoromethyl)imidazo[1,2-a]pyridine

A mixture of 3-(2-isopropyl-8-(trifluoromethyl)imidazo[1,2-a]pyridin-3-yl)phenol (96 mg, 0.30 mmol), 1-(bromomethyl)-3-(methylsulfonyl)benzene (90 mg, 0.36 mmol), and cesium carbonate (137 mg, 0.42 mmol) in DMF (2.0 mL) was heated at 40° C. under nitrogen. After 18 h, the reaction was treated with water (8 mL) and brine (2 mL). The mixture was extracted with ethyl acetate (2×10 mL). The extracts were dried (MgSO₄), concentrated in vacuo, and chromatographed on silica gel using a 30:70 to 80:20 E:H gradient to afford the title compound as an off-white solid (145 mg). MS (ES) m/z 489.1. HRMS: calcd for C₂₅H₂₃F₃N₂O₃S+H+, 489.14542. found (ESI, [M+H]+ Obs'd), 489.1461.

### Example 53

### 8-chloro-2-isopropyl-3-(3-{[3-(methylsulfonyl)benzyl]oxy}phenyl)imidazo[1,2-a]pyridine

In essentially the same manner as Example 52 but using 3-(8-chloro-2-isopropylimidazo[1,2-a]pyridin-3-yl)phenol and 1-(bromomethyl)-3-(methylsulfonyl)benzene as starting materials was prepared the title compound as an off-white solid. MS (ES) m/z 455.0. HRMS: calcd for C₂₄H₂₃ClN₂O₃S+H+, 455.11907. found (ESI, [M+H]+ Obs'd), 455.1197.

### Example 54

### 2-ethyl-3-(3-{[3-(methylsulfonyl)benzyl]oxy}phenyl)imidazo[1,2-a]pyridine-8-carbonitrile

In essentially the same manner as Example 52 but using 3-(8-cyano-2-ethylimidazo[1,2-a]pyridin-3-yl)phenol and 1-(bromomethyl)-3-(methylsulfonyl)benzene as starting materials was prepared the title compound as a yellow solid. MS (ES) m/z 432.0. HRMS: calcd for C₂₄H₂₁N₃O₃S+H+, 432.13764. found (ESI, [M+H]+ Obs'd), 432.1383.

### Example 55

### 2-isopropyl-3-(3-{[3-(methylsulfonyl)benzyl]oxy}phenyl)imidazo[1,2-a]pyridine-8-carbonitrile

In essentially the same manner as Example 52 but using 3-(8-cyano-2-isopropylimidazo[1,2-a]pyridin-3-yl)phenol and 1-(bromomethyl)-3-(methylsulfonyl)benzene as starting materials was prepared the title compound as a yellow solid. MS (ES) m/z 446.1. HRMS: calcd for C₂₅H₂₃N₃O₃S+H+, 446.15329. found (ESI, [M+H]+ Obs'd), 446.1545.

### Example 56

### 2-isopropyl-3-(3-{[4-(methylsulfonyl)benzyl]oxy}phenyl)-8-(trifluoromethyl)imidazo[1,2-a]pyridine

In essentially the same manner as Example 52 but using 3-(2-isopropyl-8-(trifluoromethyl)imidazo[1,2-a]pyridin-3-yl)phenol and 1-(chloromethyl)-3-(methylsulfonyl)benzene as starting materials was prepared the title compound as a white solid. MS (ES) m/z 489.1. HRMS: calcd for C₂₅H₂₃F₃N₂O₃S+Na+, 511.12736. found (ESI, [M+Na]+ Obs'd), 511.1280.

### Example 57

### 8-chloro-2-isopropyl-3-(3-{[4-(methylsulfonyl)benzyl]oxy}phenyl)imidazo[1,2-a]pyridine

In essentially the same manner as Example 52 but using 3-(8-chloro-2-isopropylimidazo[1,2-a]pyridin-3-yl)phenol and 1-(chloromethyl)-3-(methylsulfonyl)benzene as starting materials was prepared the title compound as an off-white solid. MS (ES) m/z 455.0. HRMS: calcd for C₂₄H₂₃ClN₂O₃S+H+, 455.11907. found (ESI, [M+H]+ Obs'd), 455.1195.

### Example 58

### 2-ethyl-3-(3-{[4-(methylsulfonyl)benzyl]oxy}phenyl)imidazo[1,2-a]pyridine-8-carbonitrile

In essentially the same manner as Example 52 but using 3-(8-cyano-2-ethylimidazo[1,2-a]pyridin-3-yl)phenol and 1-(chloromethyl)-3-(methylsulfonyl)benzene as starting materials was prepared the title compound as a yellow solid. MS (ES) m/z 432.1. HRMS: calcd for C₂₄H₂₁N₃O₃S+H+, 432.13764. found (ESI, [M+H]+ Obs'd), 432.1383.

### Example 59

### 2-isopropyl-3-(3-{[4-(methylsulfonyl)benzyl]oxy}phenyl)imidazo[1,2-a]pyridine-8-carbonitrile

In essentially the same manner as Example 52 but using 3-(8-cyano-2-isopropylimidazo[1,2-a]pyridin-3-yl)phenol and 1-(chloromethyl)-3-(methylsulfonyl)benzene as starting materials was prepared the title compound as a yellow solid. MS (ES) m/z 446.1. HRMS: calcd for C₂₅H₂₃N₃O₃S+H+, 446.15329. found (ESI, [M+H]+ Obs'd), 446.1543.

### Example 60

### 2-isopropyl-8-(trifluoromethyl)-3-(3-{3-[(trifluoromethyl)sulfonyl]phenoxy}phenyl)imidazo[1,2-a]pyridine

### Step 1) 3-fluorobenzenesulfonyl fluoride

A mixture of 3-fluorobenzene-1-sulfonyl chloride (5.00 g, 25.7 mmol), 18-crown-6 (0.170 g, 0.642 mmol) and potassium fluoride (7.46 g, 128 mmol) in acetonitrile (51 mL) was stirred for 4 h. Saturated NaHCO₃ (200 mL) was added and the mixture was extracted with ethyl acetate. The combined organics were washed with saturated NaHCO₃ (200 mL) and dried over MgSO₄ and concentrated to yield the title intermediate as a yellow oil that was used without purification in the next step (4.20 g, 92%).

### Step 2) 1-fluoro-3-trifluoromethanesulfonyl-benzene

3-Fluorobenzenesulfonyl fluoride (4.20 g, 23.6 mmol) and tris(dimethylamino)sulfur(trimethylsilyl)difluoride (0.649 g, 2.36 mmol) was taken into THF (24 mL). (Trifluoromethyl)trimethylsilane (7.06 mL, 47.1 mmol) in THF (24 mL) was added dropwise over 15 min. The reaction was allowed to stir overnight at room temperature. Water was added and then the mixture was extracted with ethyl acetate. The combined extracts were washed several times with water, then by half-saturated brine, dried over MgSO₄ and concentrated. The crude product was purified by column chromatography using a 0:100 to 20:80 E:H gradient to provide the title intermediate as a yellow liquid (3.32 g, 62%).

### Step 3) 2-isopropyl-8-(trifluoromethyl)-3-(3-{3-[(trifluoromethyl)sulfonyl]phenoxy}phenyl)imidazo[1,2-a]pyridine

A mixture of 3-(2-isopropyl-8-(trifluoromethyl)imidazo[1,2-a]pyridin-3-yl)phenol (0.100 g, 0.312 mmol), 1-fluoro-3-(trifluoromethylsulfonyl)benzene (0.142 g, 0.624 mmol) and potassium carbonate (0.086 g, 0.62 mmol) in DMF (3 mL) was heated at 100° C. overnight. The mixture was cooled, then diluted with water. The mixture was extracted with ethyl acetate and the combined organics were washed with half-saturated brine and dried over MgSO₄. Purification by column chromatography using a 0:100 to 20:80 E:H gradient afforded the title compound as a white solid (0.121 g, 73%). HRMS: calcd for C₂₄H₁₈F₆N₂O₃S+H+, 529.10151. found (ESI, [M+H]+ Obs'd), 529.1020.

### Example 61

### 3-[(3-{3-[2-isopropyl-8-(trifluoromethyl)imidazo[1,2-a]pyridin-3-yl]phenoxy}phenyl)sulfonyl]propan-1-ol

The title compound was prepared in a similar manner to that of Example 25 except using 3-(3-bromophenylsulfonyl)propan-1-ol in place of 1-bromo-3-(methylsulfonyl)benzene. MS (ES) m/z 519.1. HRMS: calcd for C₂₆H₂₅F₃N₂O₄S+H+, 519.15599. found (ESI, [M+H]+ Obs'd), 519.1566.

### Example 62

### Step 1) 3-(3-(3-(2-isopropyl-8-(trifluoromethyl)imidazo[1,2-a]pyridin-3-yl)phenoxy)phenylsulfonyl)propyl methanesulfonate

3-(3-(3-(2-isopropyl-8-(trifluoromethyl)imidazo[1,2-a]pyridin-3-yl)phenoxy)phenylsulfonyl)propan-1-ol (0.431 g, 0.83 mmol) and triethylamine (0.23 mL, 1.66 mmol) in dichloromethane (5 mL) was cooled to 0° C. Methanesulfonyl chloride (0.129 mL, 1.66 mmol) was added dropwise and the reaction stirred overnight at room temperature. Saturated aqueous NaHCO₃ was added and the mixture extracted with dichloromethane and concentrated to afford the title intermediate used in the next step without purification (0.571 g).

### Step 2) 3-[(3-{3-[2-isopropyl-8-(trifluoromethyl)imidazo[1,2-a]pyridin-3-yl]phenoxy}phenyl)sulfonyl]-N-methylpropan-1-amine

A mixture of 3-(3-(3-(2-isopropyl-8-(trifluoromethyl)imidazo[1,2-a]pyridin-3-yl)phenoxy)phenylsulfonyl)propyl methanesulfonate (0.250 g, 0.419 mmol) and 2.0 M methylamine in MeOH (2.09 mL, 4.19 mmol) in THF (5 mL) was heated at 60° C. overnight. The reaction mixture was concentrated and purified via column chromatography using a 0:100 to 15:85 MeOH:DCM gradient to provide impure product that was further purified via reverse phase chromatography using a 0:100 to 100:0 acetonitrile:water gradient to afford the title compound as an off-white solid (0.100 g, 45%). MS (ES) m/z 531.8. MS (ES) m/z 531.8; HRMS: calcd for C₂₇H₂₈F₃N₃O₃S+H+, 532.18762. found (ESI, [M+H]+ Obs'd), 532.1879.

### Example 63

### Step 1) 8-chloro-2-ethylimidazo[1,2-a]pyridine

A mixture of 3-chloropyridin-2-amine (3.00 g, 23.3 mmol), 1-bromobutan-2-one (2.62 mL, 25.7 mmol) and sodium bicarbonate (3.92 g, 46.7 mmol) in ethanol (50 mL) was refluxed overnight. The ethanol was removed and the resulting material was taken into ethyl acetate and water and the layers separated. The aqueous layer was extracted with ethyl acetate and the combined organics were dried over MgSO₄ and concentrated. Purification by column chromatography using a 5:95 to 40:60 E:H gradient afforded the title intermediate (3.56 g, 84%).

### Step 2) 3-(8-chloro-2-ethylimidazo[1,2-a]pyridin-3-yl)phenol

A mixture of 8-chloro-2-ethylimidazo[1,2-a]pyridine (1.00 g, 5.54 mmol), 3-iodophenol (1.34 g, 6.09 mmol), potassium acetate (1.63 g, 16.6 mmol) and 20% palladium hydroxide on carbon powder (0.389 g, 0.554 mmol) in N,N-dimethylacetamide (30 mL) was heated at 145° C. overnight. The reaction was cooled and filtered through Celite®. The filtrate was treated water and extracted with ethyl acetate. The combined extracts were washed with half-saturated brine and dried over MgSO₄. Purification via column chromatography eluting with a 0:100 to 50:50 E:H gradient afforded the title intermediate (0.97 g, 64%).

### Step 3) 8-chloro-2-ethyl-3-{3-[3-(methylsulfonyl)phenoxy]phenyl}imidazo[1,2-a]pyridine

The title compound was prepared in a similar manner to that of Example 25 except using 3-(8-chloro-2-ethylimidazo[1,2-a]pyridin-3-yl)phenol in place of 3-(2-isopropyl-8-(trifluoromethyl)imidazo[1,2-a]pyridin-3-yl)phenol. MS (ES) m/z 426.9. HRMS: calcd for C₂₂H₁₉ClN₂O₃S+H+, 427.08777. found (ESI, [M+H]+ Obs'd), 427.0878.

### Example 64

### 8-chloro-2-ethyl-3-{3-[3-(ethylsulfonyl)phenoxy]phenyl}imidazo[1,2-a]pyridine

The title compound was prepared in a similar manner to that of Example 25 except using 3-(8-chloro-2-ethylimidazo[1,2-a]pyridin-3-yl)phenol in place of 3-(2-isopropyl-8-(trifluoromethyl)imidazo[1,2-a]pyridin-3-yl)phenol and 1-bromo-3-(ethylsulfonyl)benzene in place of 1-bromo-3-(methylsulfonyl)benzene. MS (ES) m/z 440.9. HRMS: calcd for C₂₃H₂₁ClN₂O₃S+H+, 441.10342. found (ESI, [M+H]+ Obs'd), 441.1049.

### Example 65

### 8-chloro-2-ethyl-3-{3-[3-(isopropylsulfonyl)phenoxy]phenyl}imidazo[1,2-a]pyridine

The title compound was prepared in a similar manner to that of Example 25 except using 3-(8-chloro-2-ethylimidazo[1,2-a]pyridin-3-yl)phenol in place of 3-(2-isopropyl-8-(trifluoromethyl)imidazo[1,2-a]pyridin-3-yl)phenol and 1-bromo-3-(isopropylsulfonyl)benzene in place of 1-bromo-3-(methylsulfonyl)benzene. MS (ES) m/z 454.9. HRMS: calcd for C₂₄H₂₃ClN₂O₃S+H+, 455.11907. found (ESI, [M+H]+ Obs'd), 455.1196.

### Example 66

### Step 1) 8-chloro-2-isopropylimidazo[1,2-a]pyridine

A mixture of 3-chloropyridin-2-amine (3.00 g, 23.34 mmol), 1-bromo-3-methylbutan-2-one (4.24 g, 25.7 mmol) and sodium bicarbonate (3.92 g, 46.7 mmol) in ethanol (75 mL) was heated at reflux overnight. The ethanol was removed and the resulting material was taken into ethyl acetate and water and the layers were separated. The aqueous layer was extracted with additional ethyl acetate. The combined organics were dried over MgSO₄ and concentrated. Purification via column chromatography using 0:100 to 40:60 E:H gradient afforded 2.82 g (62%) of the title intermediate.

### Step 2) 3-(8-chloro-2-isopropylimidazo[1,2-a]pyridin-3-yl)phenol

A mixture of 8-chloro-2-isopropylimidazo[1,2-a]pyridine (2.74 g, 14.1 mmol), 3-iodophenol (3.41 g, 15.5 mmol), potassium acetate (4.14 g, 42.2 mmol) and 20% palladium hydroxide on carbon powder (0.988 g, 1.41 mmol) in N,N-dimethylacetamide (70 mL) was heated at 145° C. overnight. The reaction was cooled filtered through Celite®. Ethyl acetate and water were added and the layers were separated. The aqueous layer was extracted with additional ethyl acetate. The combined extracts were washed with half-saturated brine, dried over MgSO₄, and concentrated. Purification via column chromatography using E:H afforded 3.48 g (86%) of the title intermediate as an off-white solid. MS (ES) m/z 287.0. HRMS: calcd for C₁₆H₁₅ClN₂O+H+, 287.09457. found (ESI, [M+H]+ Obs'd), 287.0938.

### Step 3) 8-chloro-2-isopropyl-3-{3-[3-(methylsulfonyl)phenoxy]phenyl}imidazo[1,2-a]pyridine

The title compound was prepared in a similar manner to that of Example 25 except using 3-(8-chloro-2-isopropylimidazo[1,2-a]pyridin-3-yl)phenol in place of 3-(2-isopropyl-8-(trifluoromethyl)imidazo[1,2-a]pyridin-3-yl)phenol. MS (ES) m/z 441.2. MS (ES) m/z 441.2; HRMS: calcd for C₂₃H₂₁ClN₂O₃S+H+, 441.10342. found (ESI, [M+H]+ Obs'd), 441.1048.

### Example 67

### 8-chloro-3-{3-[3-(ethylsulfonyl)phenoxy]phenyl}-2-isopropylimidazo[1,2-a]pyridine

The title compound was prepared in a similar manner to that of Example 25 except using 3-(8-chloro-2-isopropylimidazo[1,2-a]pyridin-3-yl)phenol in place of 3-(2-isopropyl-8-(trifluoromethyl)imidazo[1,2-a]pyridin-3-yl)phenol and 1-bromo-3-(ethylsulfonyl)benzene in place of 1-bromo-3-(methylsulfonyl)benzene. MS (ES) m/z 454.9. HRMS: calcd for C₂₄H₂₃ClN₂O₃S+H+, 455.11907. found (ESI, [M+H]+ Obs'd), 455.1197.

### Example 68

### 8-chloro-2-isopropyl-3-{3-[3-(isopropylsulfonyl)phenoxy]phenyl}imidazo[1,2-a]pyridine

The title compound was prepared in a similar manner to that of Example 25 except using 3-(8-chloro-2-isopropylimidazo[1,2-a]pyridin-3-yl)phenol in place of 3-(2-isopropyl-8-(trifluoromethyl)imidazo[1,2-a]pyridin-3-yl)phenol and 1-bromo-3-(isopropylsulfonyl)benzene in place of 1-bromo-3-(methylsulfonyl)benzene. MS (ES) m/z 468.6. HRMS: calcd for C₂₅H₂₅ClN₂O₃S+H+, 469.13472. found (ESI, [M+H]+ Obs'd), 469.1351.

### Example 69

### Step 1) 2-ethylimidazo[1,2-a]pyridine-8-carbonitrile

A mixture 2-aminonicotinonitrile (5.00 g, 42.0 mmol), 1-bromobutan-2-one (4.71 mL, 46.2 mmol) and sodium bicarbonate (7.05 g, 84.0 mmol) in ethanol (100 mL) was heated at reflux overnight. The ethanol was removed in vacuo and the resulting material was taken into water and ethyl acetate. The layers were separated and the aqueous layer was extracted several times with ethyl acetate. The combined extracts were dried over MgSO₄ and concentrated. Purification via column chromatography eluting with a 10:90 to 100:0 E:H gradient afforded 4.62 g (64%) of the title intermediate as a white solid.

### Step 2) 2-ethyl-3-(3-hydroxyphenyl)imidazo[1,2-a]pyridine-8-carbonitrile

A mixture of 2-ethylimidazo[1,2-a]pyridine-8-carbonitrile (1.79 g, 10.5 mmol), 3-iodophenol (2.53 g, 11.5 mmol), potassium acetate (3.08 g, 31.4 mmol) and 20% palladium hydroxide (0.147 g, 1.046 mmol) in N,N-dimethylacetamide (50 mL) was heated at 145° C. overnight. The reaction was cooled and filtered through Celite®. The filtrate was partitioned between ethyl acetate and water and the layers separated. The aqueous layer was extracted with ethyl acetate. The combined extracts were washed with half-saturated brine, dried over MgSO₄, and concentrated. Purification via column chromatography eluting with a 5:95 to 45:55 E:H gradient afforded the title intermediate (2.47 g, 90%) as a tan solid.

### Step 3) 2-ethyl-3-{3-[3-(methylsulfonyl)phenoxy]phenyl}imidazo[1,2-a]pyridine-8-carbonitrile

The title compound was prepared in a similar manner to that of Example 25 except using 2-ethyl-3-(3-hydroxyphenyl)imidazo[1,2-a]pyridine-8-carbonitrile in place of 3-(2-isopropyl-8-(trifluoromethyl)imidazo[1,2-a]pyridin-3-yl)phenol MS (ES) m/z 418.0. HRMS: calcd for C₂₃H₁₉N₃O₃S+H+, 418.12199. found (ESI, [M+H]+ Obs'd), 418.1218.

### Example 70

### 2-ethyl-3-{3-[3-(ethylsulfonyl)phenoxy]phenyl}imidazo[1,2-a]pyridine-8-carbonitrile

The title compound was prepared in a similar manner to that of Example 25 except using 2-ethyl-3-(3-hydroxyphenyl)imidazo[1,2-a]pyridine-8-carbonitrile in place of 3-(2-isopropyl-8-(trifluoromethyl)imidazo[1,2-a]pyridin-3-yl)phenol and 1-bromo-3-(ethylsulfonyl)benzene in place of 1-bromo-3-(methylsulfonyl)benzene. MS (ES) m/z 432.1. HRMS: calcd for C₂₄H₂₁N₃O₃S+H+, 432.13764. found (ESI, [M+H]+ Obs'd), 432.1389.

### Example 71

### 2-ethyl-3-{3-[3-(isopropylsulfonyl)phenoxy]phenyl}imidazo[1,2-a]pyridine-8-carbonitrile

The title compound was prepared in a similar manner to that of Example 25 except using 2-ethyl-3-(3-hydroxyphenyl)imidazo[1,2-a]pyridine-8-carbonitrile in place of 3-(2-isopropyl-8-(trifluoromethyl)imidazo[1,2-a]pyridin-3-yl)phenol and 1-bromo-3-(isopropylsulfonyl)benzene in place of 1-bromo-3-(methylsulfonyl)benzene. MS (ES) m/z 445.9. HRMS: calcd for C₂₅H₂₃N₃O₃S+H+, 446.15329. found (ESI, [M+H]+ Obs'd), 446.1539.

### Example 72

### Step 1) 2-isopropylimidazo[1,2-a]pyridine-8-carbonitrile

A mixture of 2-aminonicotinonitrile (5.51 g, 46.3 mmol), 1-bromo-3-methylbutan-2-one (9.16 g, 55.5 mmol) and sodium bicarbonate (7.77 g, 93 mmol) in ethanol (150 mL) was refluxed for 65 h. The ethanol was removed and the resulting material was taken into water and ethyl acetate. The layers were separated and the aqueous layer was extracted with ethyl acetate. The combined organic extracts were dried over MgSO₄ and concentrated. Purification via column chromatography eluting with a 10:90 to 100:0 E:H gradient afforded 3.33 g (39%) of the title intermediate.

### Step 2) 3-(3-hydroxyphenyl)-2-isopropylimidazo[1,2-a]pyridine-8-carbonitrile

A mixture of 2-isopropylimidazo[1,2-a]pyridine-8-carbonitrile (1.70 g, 9.18 mmol), 3-iodophenol (2.22 g, 10.1 mmol), potassium acetate (2.70 g, 27.5 mmol) and 20% palladium hydroxide on carbon (0.644 g, 0.918 mmol) in N,N-dimethylacetamide (46 mL) was heated at 145° C. overnight. The reaction was cooled and filtered through Celite®. The filtrate was partitioned between ethyl acetate and water and the layers separated. The aqueous layer was extracted with ethyl acetate. The combined organic extracts were washed with half-saturated brine and dried over MgSO₄. Purification via column chromatography eluting with a 5:95 to 45:55 E:H gradient afforded the title intermediate (1.93 g, 76%).

### Step 3) 2-isopropyl-3-{3-[3-(methylsulfonyl)phenoxy]phenyl}imidazo[1,2-a]pyridine-8-carbonitrile

The title compound was prepared in a similar manner to that of Example 25 except using 3-(3-hydroxyphenyl)-2-isopropylimidazo[1,2-a]pyridine-8-carbonitrile in place of 3-(2-isopropyl-8-(trifluoromethyl)imidazo[1,2-a]pyridin-3-yl)phenol. MS (ES) m/z 432.2. HRMS: calcd for C₂₄H₂₁N₃O₃S+H+, 432.13764. found (ESI, [M+H]+ Obs'd), 432.1381.

### Example 73

### 3-{3-[3-(ethylsulfonyl)phenoxy]phenyl}-2-isopropylimidazo[1,2-a]pyridine-8-carbonitrile

The title compound was prepared in a similar manner to that of Example 25 except using 3-(3-hydroxyphenyl)-2-isopropylimidazo[1,2-a]pyridine-8-carbonitrile in place of 3-(2-isopropyl-8-(trifluoromethyl)imidazo[1,2-a]pyridin-3-yl)phenol and 1-bromo-3-(ethylsulfonyl)benzene in place of 1-bromo-3-(methylsulfonyl)benzene. MS (ES) m/z 446.1. HRMS: calcd for C₂₅H₂₃N₃O₃S+H+, 446.15329. found (ESI, [M+H]+ Obs'd), 446.1532.

### Example 74

### 2-isopropyl-3-{3-[3-(isopropylsulfonyl)phenoxy]phenyl}imidazo[1,2-a]pyridine-8-carbonitrile

The title compound was prepared in a similar manner to that of Example 25 except using 3-(3-hydroxyphenyl)-2-isopropylimidazo[1,2-a]pyridine-8-carbonitrile in place of 3-(2-isopropyl-8-(trifluoromethyl)imidazo[1,2-a]pyridin-3-yl)phenol and 1-bromo-3-(isopropylsulfonyl)benzene in place of 1-bromo-3-(methylsulfonyl)benzene. MS (ES) m/z 459.9. HRMS: calcd for C₂₆H₂₅N₃O₃S+H+, 460.16894. found (ESI, [M+H]+ Obs'd), 460.1697.

### Example 75

### Step 1) 2-tert-butyl-8-(trifluoromethyl)imidazo[1,2-a]pyridine

A mixture of 3-(trifluoromethyl)pyridin-2-amine (1.50 g, 9.25 mmol), 1-bromo-3,3-dimethylbutan-2-one (1.25 mL, 9.25 mmol) and sodium bicarbonate (1.56 g, 18.5 mmol) in ethanol (31 mL) was refluxed for 88 h. The ethanol was removed and to the resulting material was added water. The mixture was extracted with DCM and dried over MgSO₄. Purification via column chromatography using a 0:100 to 20:80 E:H gradient afforded the title intermediate (0.59 g, 26%) as an off-white solid.

### Step 2) 2-tert-butyl-3-{3-[3-(methylsulfonyl)phenoxy]phenyl}-8-(trifluoromethyl)imidazo[1,2-a]pyridine

A mixture of 2-tert-butyl-8-(trifluoromethyl)imidazo[1,2-a]pyridine (0.100 g, 0.413 mmol), 1-iodo-3-(3-(methylsulfonyl)phenoxy)benzene (0.170 g, 0.454 mmol), potassium acetate (0.122 g, 1.24 mmol) and 20% palladium hydroxide on carbon (0.029 g, 0.041 mmol) in N,N-dimethylacetamide (2 mL) was heated at 145° C. overnight. The reaction was cooled and filtered through Celite®. The resulting material was partitioned between ethyl acetate and water and the layers were separated. The organic layer was washed several times with water then with a solution of half-saturated brine. The combined extracts were dried over MgSO₄ and concentrated. Purification via column chromatography eluting with a 0:100 to 25:75 E:H gradient afforded the title compound (0.144 g, 71%) as a white solid. MS (ES) m/z 488.9. HRMS: calcd for C₂₅H₂₃F₃N₂O₃S+H+, 489.14542. found (ESI, [M+H]+ Obs'd), 489.1448.

### Example 76

### Step 1) 3-(2-tert-butyl-8-(trifluoromethyl)imidazo[1,2-a]pyridin-3-yl)phenol

A mixture of 2-tert-butyl-8-(trifluoromethyl)imidazo[1,2-a]pyridine (0.399 g, 1.65 mmol), 3-iodophenol (0.399 g, 1.81 mmol), potassium acetate (0.485 g, 4.94 mmol) and 20% palladium hydroxide on carbon (0.116 g, 0.165 mmol) in N,N-dimethylacetamide (20 mL) was heated at 145° C. overnight. The reaction was cooled and filtered through Celite®. The resulting material was partitioned between ethyl acetate and water and the layers were separated. The organic layer was washed several times with water then with a solution of half-saturated brine. The combined extracts were dried over MgSO₄ and concentrated. Purification via column chromatography eluting with a 0:100 to 30:70 E:H gradient afforded the title intermediate (0.372 g, 68%).

### Step 2) 2-tert-butyl-3-{3-[3-(ethylsulfonyl)phenoxy]phenyl}-8-(trifluoromethyl)imidazo[1,2-a]pyridine

The title compound was prepared in a similar manner to that of Example 25 except using 3-(2-tert-butyl-8-(trifluoromethyl)imidazo[1,2-a]pyridin-3-yl)phenol in place of 3-(2-isopropyl-8-(trifluoromethyl)imidazo[1,2-a]pyridin-3-yl)phenol and 1-bromo-3-(ethylsulfonyl)benzene in place of 1-bromo-3-(methylsulfonyl)benzene. MS (ES) m/z 503.1. HRMS: calcd for C₂₆H₂₅F₃N₂O₃S+H+, 503.16107. found (ESI, [M+H]+ Obs'd), 503.1615.

### Example 77

### 2-tert-butyl-3-{3-[3-(isopropylsulfonyl)phenoxy]phenyl}-8-(trifluoromethyl)imidazo[1,2-a]pyridine

The title compound was prepared in a similar manner to that of Example 25 except using 3-(2-tert-butyl-8-(trifluoromethyl)imidazo[1,2-a]pyridin-3-yl)phenol in place of 3-(2-isopropyl-8-(trifluoromethyl)imidazo[1,2-a]pyridin-3-yl)phenol and 1-bromo-3-(isopropylsulfonyl)benzene in place of 1-bromo-3-(methylsulfonyl)benzene.

¹H NMR (400 MHz, DMSO-d⁶) δ 1.13 (d, 6H), 1.25 (s, 9H), 3.4 (m, 1H), 6.9 (t, 1H), 7.25-7.55 (m, 5H), 7.6-7.75 (m, 4H), 7.95 (d, 1H).

### Example 78

### 3-[(3-{3-[2-tert-butyl-8-(trifluoromethyl)imidazo[1,2-a]pyridin-3-yl]phenoxy}phenyl)sulfonyl]propan-1-ol

The title compound was prepared in a similar manner to that of Example 25 except using 3-(2-tert-butyl-8-(trifluoromethyl)imidazo[1,2-a]pyridin-3-yl)phenol in place of 3-(2-isopropyl-8-(trifluoromethyl)imidazo[1,2-a]pyridin-3-yl)phenol and 3-(3-bromophenylsulfonyl)propan-1-ol in place of 1-bromo-3-(methylsulfonyl)benzene. MS (ES) m/z 533.0. HRMS: calcd for C₂₇H₂₇F₃N₂O₄S+H+, 533.17164. found (ESI, [M+H]+ Obs'd), 533.1724.

### Example 79

### Step 1) 2-tert-butylimidazo[1,2-a]pyridine-8-carbonitrile

A mixture of 2-aminonicotinonitrile (3.00 g, 25.2 mmol) and 1-bromo-3,3-dimethylbutan-2-one (4.07 mL, 30.2 mmol) in ethanol (84 mL) was refluxed overnight. The ethanol was removed. The resulting material was partitioned between water and ethyl acetate. The layers were separated and the aqueous layer extract with additional ethyl acetate. The combined extracts were dried over MgSO₄ and concentrated. Purification via column chromatography using E:H gradient afforded an off-white solid 1.66 g (33%).

### Step 2) 2-tert-butyl-3-(3-hydroxyphenyl)imidazo[1,2-a]pyridine-8-carbonitrile

A mixture of 2-tert-butylimidazo[1,2-a]pyridine-8-carbonitrile (1.60 g, 8.03 mmol), 3-iodophenol (1.943 g, 8.83 mmol), potassium acetate (2.364 g, 24.09 mmol) and 20% palladium hydroxide on carbon powder (0.564 g, 0.803 mmol) in N,N-dimethylacetamide (40 mL) was heated at 145° C. overnight. The reaction was cooled then filtered through Celite® using ethyl acetate to transfer. The mother liquor was further diluted with ethyl acetate and washed with water twice, then with brine. The organic layer was dried over MgSO₄ and concentrated. Purification via column chromatography using a 0:100 to 70:30 E:H gradient produced the title intermediate as a light yellow solid.

### Step 3) 2-tert-butyl-3-{3-[3-(ethylsulfonyl)phenoxy]phenyl}imidazo[1,2-a]pyridine-8-carbonitrile

The title compound was prepared in a similar manner to that of Example 25 except using 2-tert-butyl-3-(3-hydroxyphenyl)imidazo[1,2-a]pyridine-8-carbonitrile in place of 3-(2-isopropyl-8-(trifluoromethyl)imidazo[1,2-a]pyridin-3-yl)phenol and 1-bromo-3-(ethylsulfonyl)benzene in place of 1-bromo-3-(methylsulfonyl)benzene. MS (ES) m/z 460.1. HRMS: calcd for C₂₆H₂₅N₃O₃S+H+, 460.16894. found (ESI, [M+H]+ Obs'd), 460.1691.

### Example 80

### 2-tert-butyl-3-{3-[3-(isopropylsulfonyl)phenoxy]phenyl}imidazo[1,2-a]pyridine-8-carbonitrile

The title compound was prepared in a similar manner to that of Example 25 except using 2-tert-butyl-3-(3-hydroxyphenyl)imidazo[1,2-a]pyridine-8-carbonitrile in place of 3-(2-isopropyl-8-(trifluoromethyl)imidazo[1,2-a]pyridin-3-yl)phenol and 1-bromo-3-(isopropylsulfonyl)benzene was used in place of 1-bromo-3-(methylsulfonyl)benzene. MS (ES) m/z 474.1. HRMS: calcd for C₂₇H₂₇N₃O₃S+H+, 474.18459. found (ESI, [M+H]+ Obs'd), 474.1848.

### Example 81

### 2-tert-butyl-3-(3-{3-[(3-hydroxypropyl)sulfonyl]phenoxy}phenyl)imidazo[1,2-a]pyridine-8-carbonitrile

The title compound was prepared in a similar manner to that of Example 25 except using 2-tert-butyl-3-(3-hydroxyphenyl)imidazo[1,2-a]pyridine-8-carbonitrile in place of 3-(2-isopropyl-8-(trifluoromethyl)imidazo[1,2-a]pyridin-3-yl)phenol and 3-(3-bromophenylsulfonyl)propan-1-ol in place of 1-bromo-3-(methylsulfonyl)benzene. MS (ES) m/z 490.1. HRMS: calcd for C₂₇H₂₇N₃O₄S+H+, 490.17950. found (ESI, [M+H]+ Obs'd), 490.1799.

### Example 82

### Step 1) 3-(2-chloro-5-hydroxyphenyl)-2-isopropylimidazo[1,2-a]pyridine-8-carbonitrile

A mixture of 2-isopropylimidazo[1,2-a]pyridine-8-carbonitrile (0.700 g, 3.78 mmol), 3-bromo-4-chlorophenol (1.18 g, 5.67 mmol), potassium acetate (1.113 g, 11.3 mmol) and 20% palladium hydroxide on carbon powder (0.265 g, 0.378 mmol) in N,N-dimethylacetamide (20 mL) was heated at 155° C. for 65 h. The reaction was cooled and filtered through Celite®. Ethyl acetate and water were added and the layers were separated. The aqueous layer was extracted with additional ethyl acetate. The combined extracts were washed with half-saturated brine, dried over MgSO₄, and concentrated. Purification via column chromatography using an E:H gradient produced the title intermediate.

### Step 2) 3-{2-chloro-5-[3-(methylsulfonyl)phenoxy]phenyl}-2-isopropylimidazo[1,2-a]pyridine-8-carbonitrile

The title compound was prepared in a similar manner to that of Example 25 except using 3-(2-chloro-5-hydroxyphenyl)-2-isopropylimidazo[1,2-a]pyridine-8-carbonitrile in place of 3-(2-isopropyl-8-(trifluoromethyl)imidazo[1,2-a]pyridin-3-yl)phenol. MS (ESI) m/z 466. HRMS: calcd for C₂₄H₂₀ClN₃O₃S+H+, 466.09866. found (ESI, [M+H]+ Obs'd), 466.0995.

### Example 83

### 3-{2-chloro-5-[3-(ethylsulfonyl)phenoxy]phenyl}-2-isopropylimidazo[1,2-a]pyridine-8-carbonitrile

The title compound was prepared in a similar manner to that of Example 25 except using 3-(2-chloro-5-hydroxyphenyl)-2-isopropylimidazo[1,2-a]pyridine-8-carbonitrile in place of 3-(2-isopropyl-8-(trifluoromethyl)imidazo[1,2-a]pyridin-3-yl)phenol and 1-bromo-3-(ethylsulfonyl)benzene in place of 1-bromo-3-(methylsulfonyl)benzene. MS (ESI) m/z 480. HRMS: calcd for C₂₅H₂₂ClN₃O₃S+H+, 480.11431. found (ESI, [M+H]+ Obs'd), 480.1148.

### Example 84

### 3-{2-chloro-5-[3-(isopropylsulfonyl)phenoxy]phenyl}-2-isopropylimidazo[1,2-a]pyridine-8-carbonitrile

The title compound was prepared in a similar manner to that of Example 25 except using 3-(2-chloro-5-hydroxyphenyl)-2-isopropylimidazo[1,2-a]pyridine-8-carbonitrile in place of 3-(2-isopropyl-8-(trifluoromethyl)imidazo[1,2-a]pyridin-3-yl)phenol and 1-bromo-3-(isopropylsulfonyl)benzene in place of 1-bromo-3-(methylsulfonyl)benzene. MS (ES) m/z 494.1. HRMS: calcd for C₂₆H₂₄ClN₃O₃S+H+, 494.12996. found (ESI, [M+H]+ Obs'd), 494.1305.

### Example 85

### Step 1) 2-chloro-3-(3-methoxyphenyl)-8-(trifluoromethyl)imidazo[1,2-a]pyridine

A mixture of 3-(trifluoromethyl)pyridin-2-amine (2.00 g, 12.3 mmol) and 3-methoxyphenylglyoxal hydrate (2.25 g, 12.3 mmol) in dichloromethane (25 mL) was refluxed for 1 h. The solvent was removed and the resulting material was taken into carbon tetrachloride (25 mL) along with thionyl chloride (80 mL). This mixture was refluxed for 0.5 h. The solvent was removed and the resulting material was carefully neutralized with aqueous K₂CO₃. Extraction with ethyl acetate followed by drying over MgSO₄ and concentration afforded the crude product. Purification via column chromatography using 0:100 to 30:70 E:H gradient afforded 2.24 g (56%) of the title intermediate.

### Step 2) 3-(2-chloro-8-(trifluoromethyl)imidazo[1,2-a]pyridin-3-yl)phenol

A mixture of 2-chloro-3-(3-methoxyphenyl)-8-(trifluoromethyl)imidazo[1,2-a]pyridine (2.13 g, 6.52 mmol) in dichloromethane (65 mL) was cooled to 0° C. Boron trifluoride-methyl sulfide complex (4.12 mL, 39.1 mmol) was added dropwise. The reaction was then allowed to stir at room temperature overnight. Water and methanol were added. A stream of nitrogen was passed over the mixture for 1 h to remove Me₂S. The reaction was extract with ethyl acetate and the extracts drier over MgSO₄. Purification via column chromatography using 0:100 to 40:60 E:H gradient afforded 1.04 g (51%) of the title intermediate.

### Step 3) 2-chloro-3-{3-[3-(methylsulfonyl)phenoxy]phenyl}-8-(trifluoromethyl)imidazo[1,2-a]pyridine

The title compound was prepared in a similar manner to that of Example 25 except using 3-(2-chloro-8-(trifluoromethyl)imidazo[1,2-a]pyridin-3-yl)phenol in place of 3-(2-isopropyl-8-(trifluoromethyl)imidazo[1,2-a]pyridin-3-yl)phenol. MS (ES) m/z 466.6. HRMS: calcd for C₂₁H₁₄ClF₃N₂O₃S+H+, 467.04385. found (ESI, [M+H]+ Obs'd), 467.0441.

### Example 86

### 3-{3-[3-(methylsulfonyl)phenoxy]phenyl}-2-phenyl-8-(trifluoromethyl)imidazo[1,2-a]pyridine

A mixture of 2-chloro-3-(3-(3-(methylsulfonyl)phenoxy)phenyl)-8-(trifluoromethyl)imidazo[1,2-a]pyridine (0.202 g, 0.433 mmol), phenylboronic acid (0.106 g, 0.865 mmol), (tetrakistriphenyl)phosphine palladium (0.050 g, 0.043 mmol) and potassium carbonate (0.239 g, 1.73 mmol) in 1,4-dioxane (4.5 mL) and water (1.5 mL) was heated at 100° C. overnight. The reaction was cooled and diluted with water and extracted with ethyl acetate. The combined organics were dried over MgSO₄ and concentrated. Purification via column chromatography using E:H, followed by reverse phase chromatography (0:100 to 100:0 acetonitrile:water) produced the title intermediate as a white solid. HRMS: calcd for C₂₇H₁₉F₃N₂O₃S+H+, 509.11412. found (ESI, [M+H]+ Obs'd), 509.1152.

### Example 87

### Step 1) 3-(6-bromopyridin-2-yl)-2-ethyl-8-(trifluoromethyl)imidazo[1,2-a]pyridine

A mixture of 2-ethyl-8-(trifluoromethyl)imidazo[1,2-a]pyridine (0.214 g, 1.00 mmol), 2,6-dibromopyridine (0.237 g, 1.00 mmol) and cesium carbonate (0.325 g, 1.00 mmol) in DMA (2 mL) containing a catalytic amounts of palladium(II) diacetate (5 mg, 2 mol %) and triphenylphosphine (11 mg, 4 mol %) was degassed and heated at 80° C. for 1 hr. The mixture was partitioned with ethyl acetate and H₂O. Countercurrent extraction of the aqueous layer provided 0.20 g of product as a brown oil. Purified by HPLC chromatography (silica gel, methylene chloride-acetonitrile 100:0 gradient to 85:15) provided 0.119 g of the title intermediate as a white solid. MS (ES) m/z 369.6; HRMS: calcd for C₁₅H₁₁BrF₃N₃+H+, 370.01612. found (ESI, [M+H]+ Obs'd), 370.0158.

### Step 2) 3-(6-bromopyridin-3-yl)-2-ethyl-8-(trifluoromethyl)imidazo[1,2-a]pyridine

Prepared in the same manner as outlined in Example 87, step 1, from 2-ethyl-8-(trifluoromethyl)imidazo[1,2-a]pyridine (0.214 g, 1.00 mmol) and 2,5-dibromopyridine (0.237 g, 1.00 mmol) to give the title intermediate as a white solid (0.055 g). MS (ES) m/z 369.5. HRMS: calcd for C₁₅H₁₁BrF₃N₃+H+, 370.01612. found (ESI, [M+H]⁺), 370.0157.

### Step 3) 2-ethyl-3-{6-[3-(methylsulfonyl)phenyl]pyridin-3-yl}-8-(trifluoromethyl)imidazo[1,2-a]pyridine

A mixture of 3-(6-bromopyridin-3-yl)-2-ethyl-8-(trifluoromethyl)imidazo[1,2-a]pyridine (0.050 g, 0.135 mmol), 3-(methylsulfonyl)phenylboronic acid (0.028 g, 0.14 mmol), 1.0 M aqueous sodium carbonate (0.28 mL) and tetrakis-(triphenylphosphine)palladium(0) (3.0 mg) in diglyme (2 mL) was degassed and heated at 80° C. overnight. The mixture was partitioned with ethyl acetate and H₂O to provide a brown oil. Purification by HPLC chromatography (silica gel, using a 95:5 to 85:15 gradient of methylene chloride:acetonitrile) provided the title compound as a white solid (0.010 g). NMR (400 MHz, acetone d⁶) δ 1.30 (t, 3H), 2.79 (q, 2H), 3.20 (s, 3H), 7.03 (t, 1H), 7.69 (d, 1H), 7.81 (t, 1H), 8.03 (dd, 1H), 8.16 (dd, 1H), 8.30 (dd, 1H), 8.53 (d, 1H), 8.78 (m, 1H), 8.90 (m, 1H).

### Example 88

### 2-ethyl-3-{6-[3-(methylsulfonyl)phenoxy]pyridin-2-yl}-8-(trifluoromethyl)imidazo[1,2-a]pyridine

A mixture of 3-(6-bromopyridin-2-yl)-2-ethyl-8-(trifluoromethyl)imidazo[1,2-a]pyridine (0.105 g, 0.28 mmol), 3-(methylsulfonyl)phenol (0.72 g, 0.042 mmol), cesium carbonate (0.295 g, 0.84 mmol) and copper powder (2 mg) in DMF (100 mL) was heated overnight at 100° C. The reaction mixture was partitioned with ethyl acetate and H₂O. The organic phase was concentrated in vacuo and the residue purified by prep HPLC (silica gel, eluting with a 100:0 to 80:20 gradient of methylene chloride:acetonitrile) to provide the title compound as a white solid (0.072 g). MS (ES) m/z 462.0. HRMS: calcd for C₂₂H₁₈F₃N₃O₃S+H+, 462.10937. found (ESI, [M+H]+ Obs'd), 462.1098.

### Example 89

### Step 1) 3-(6-bromopyridin-2-yl)-2-isopropyl-8-(trifluoromethyl)imidazo[1,2-a]pyridine

The title intermediate was prepared in the same manner as in Example 87 except using 2-isopropyl-8-(trifluoromethyl)imidazo[1,2-a]pyridine (0.14 g, 0.61 mmol) and 2,6-dibromopyridine (0.426 g, 1.80 mmol) to give the title intermediate as a white solid (0.028 g). MS (ESI) m/z 384; HRMS: calcd for C₁₆H₁₃BrF₃N₃+H+, 384.03177. found (ESI, [M+H]+ Obs'd), 384.0325.

### Step 2) 2-isopropyl-3-{6-[3-(methylsulfonyl)phenoxy]pyridin-2-yl}-8-(trifluoromethyl)imidazo[1,2-a]pyridine

The title compound was prepared in the same manner as in Example 88 except using 3-(6-bromopyridin-2-yl)-2-isopropyl-8-(trifluoromethyl)imidazo[1,2-a]pyridine (0.050 g, 0.13 mmol) and 3-(methylsulfonyl)phenol (0.080 g, 0.46 mmol) to give the title compound as an off-white solid (0.030 g). MS (ES) m/z 476.0; HRMS: calcd for C₂₃H₂₀F₃N₃O₃S+H+, 476.12502. found (ESI, [M+H]+ Obs'd), 476.1253.

### Example 90

### 2-isopropyl-3-(3′-(methylsulfonyl)biphenyl-4-yl)-8-(trifluoromethyl)imidazo[1,2-a]pyridine

A mixture of 3-(4-bromophenyl)-2-isopropyl-8-(trifluoromethyl)imidazo[1,2-a]pyridine (0.050 g, 0.13 mmol), 3-(methylsulfonyl)phenylboronic acid (0.040 g, 0.17 mmol), 1.0 M aqueous sodium carbonate (0.30 mL, 0.30 mmol), and tetrakis(triphenylphosphine)-palladium(0) (0.020 g, 0.020 mmol) in diglyme (2 mL) was heated at 80° C. overnight. The cooled reaction was filtered through Celite®, and chromatographed using 50:50 E:H to afford the title compound as an off-white solid. (0.034 g, 53%). MS (ES) m/z 459.1; HRMS: calcd for C₂₄H₂₁F₃N₂O₂S+H+, 459.1349. found (ESI, [M+H]+ Obs'd), 459.1346.

### Example 91

### 2-isopropyl-3-(4′-(isopropylsulfonyl)biphenyl-4-yl)-8-(trifluoromethyl)imidazo[1,2-a]pyridine

The title compound was prepared in a manner similar to that described in Example 90 except 4-(isopropylsulfonyl)phenylboronic acid was used in place of 3-(methylsulfonyl)phenylboronic acid. MS (ES) m/z 486.8; HRMS: calcd for C₂₆H₂₅F₃N₂O₂S+H+, 487.1662. found (ESI, [M+H]+ Obs'd), 487.1657.

### Example 92

### 2-ethyl-3-(4′-(isopropylsulfonyl)biphenyl-4-yl)-8-(trifluoromethyl)imidazo[1,2-a]pyridine

A mixture of 3-(4-bromophenyl)-2-ethyl-8-(trifluoromethyl)imidazo[1,2-a]pyridine (0.050 g, 0.14 mmol), 4-(isopropylsulfonyl)phenylboronic acid (0.040 g, 0.17 mmol), 1.0 M aqueous sodium carbonate (0.30 mL, 0.30 mmol), and tetrakis(triphenylphosphine)-palladium(0) (0.020 g, 0.020 mmol) in diglyme (2 mL) was heated at 80° C. overnight. The cooled reaction was filtered through Celite® and chromatographed using 50:50 E:H to afford the title compound as an off-white solid. (0.036 g, 54%). MS (ES) m/z 473.1; HRMS: calcd for C₂₅H₂₃F₃N₂O₂S+H+, 473.1505. found (ESI, [M+H]+ Obs'd), 473.1502.

### Example 93

### Step 1) 3-(4-bromophenyl)-2-ethyl-8-(trifluoromethyl)imidazo[1,2-a]pyridine

The title intermediate was prepared in a similar manner to that described in Example 87 except using 1-bromo-4-iodobenzene in place of 2,6-dibromopyridine. MS (ES) m/z 368.7.

### Step 2) 2-ethyl-3-(4′-(methylsulfonyl)biphenyl-4-yl)-8-(trifluoromethyl)imidazo[1,2-a]pyridine

The title compound was prepared in a manner similar to that described in Example 92 except using 4-(methylsulfonyl)phenylboronic acid in place of 4-(isopropylsulfonyl)phenylboronic acid. MS (ES) m/z 445.0; ¹H NMR (400 MHz, DMSO-d⁶) δ 1.29 (t, 3H), 2.80 (q, 2H), 3.16 (s, 3H), 7.00 (t, 1H), 7.65 (d, 1H), 7.72 (d, 2H), 7.99 (d, 2H), 8.01 (d, 2H), 8.06 (d, 2H), 8.49 (d, 1H).

### Example 94

### Step 1) 3-(4-bromophenyl)-2-isopropyl-8-(trifluoromethyl)imidazo[1,2-a]pyridine

The title intermediate was prepared in a similar manner to that described in Example 87 except using 2-isopropyl-8-(trifluoromethyl)imidazo[1,2-a]pyridine in place of 2-ethyl-8-(trifluoromethyl)imidazo[1,2-a]pyridine and 1-bromo-4-iodobenzene in place of 2,6-dibromopyridine. MS (ES) m/z 382.5.

### Step 2) 2-isopropyl-3-(4′-(methylsulfonyl)biphenyl-4-yl)-8-(trifluoromethyl)imidazo[1,2-a]pyridine

The title compound was prepared in a manner similar to that described in Example 90 except using 4-(methylsulfonyl)phenylboronic acid in place of 3-(methylsulfonyl)phenylboronic acid. MS (ES) m/z 459.1; HRMS: calcd for C₂₄H₂₁F₃N₂O₂S+H+, 459.1349. found (ESI, [M+H]+ Obs'd), 459.1347.

### Example 95

### 2-ethyl-3-(3′-(methylsulfonyl)biphenyl-4-yl)-8-(trifluoromethyl)imidazo[1,2-a]pyridine

The title compound was prepared in a manner similar to that described in Example 92 except using 3-(methylsulfonyl)phenylboronic acid in place of 4-(isopropylsulfonyl)phenylboronic acid. MS (ES) m/z 445.0; HRMS: calcd for C₂₃H₁₉F₃N₂O₂S+H+, 445.1192. found (ESI, [M+H]+ Obs'd), 445.1189.

### Example 96

### 4-[3-(8-chloro-2-isopropylimidazo[1,2-a]pyridin-3-yl)phenoxy]-N,N-bis(4-methoxybenzyl)benzenesulfonamide

In essentially the same manner as Example 37 but using 3-(2-isopropyl-8-chloro-imidazo[1,2-a]pyridin-3-yl)phenol and 4-fluoro-N,N-bis(4-methoxybenzyl)benzenesulfonamide as starting materials was prepared the title compound as an off-white solid. MS (ES) m/z 681.8; HRMS: calcd for C₃₈H₃₆ClN₃O₅S+H+, 682.21370. found (ESI, [M+H]+ Obs'd), 682.2136.

### Example 97

### 3-[3-(8-chloro-2-isopropylimidazo[1,2-a]pyridin-3-yl)phenoxy]-N,N-bis(4-methoxybenzyl)benzenesulfonamide

In essentially the same manner as Example 36 but using 3-(2-isopropyl-8-chloro-imidazo[1,2-a]pyridin-3-yl)phenol and 3-bromo-N,N-bis(4-methoxybenzyl)benzenesulfonamide as starting materials was prepared the title compound as an off-white solid. MS (ES) m/z 681.8; HRMS: calcd for C₃₈H₃₆ClN₃O₅S+H+, 682.21370. found (ESI, [M+H]+ Obs'd), 682.2138.

### Example 98

### 3-[3-(8-chloro-2-isopropylimidazo[1,2-a]pyridin-3-yl)phenoxy]benzenesulfonamide

In essentially the same manner as Example 38 but using 3-[3-(8-chloro-2-isopropylimidazo[1,2-a]pyridin-3-yl)phenoxy]-N,N-bis(4-methoxybenzyl)benzenesulfonamide as starting material was prepared the title compound as a white solid. MS (ES) m/z 441.7; HRMS: calcd for C₂₂H₂₀ClN₃O₃S+H+, 442.09866. found (ESI, [M+H]+ Obs'd), 442.0994.

### Example 99

### 4-[3-(8-chloro-2-isopropylimidazo[1,2-a]pyridin-3-yl)phenoxy]benzenesulfonamide

In essentially the same manner as Example 38 but using 4-[3-(8-chloro-2-isopropylimidazo[1,2-a]pyridin-3-yl)phenoxy]-N,N-bis(4-methoxybenzyl)benzenesulfonamide as starting material was prepared the title compound as a white solid. MS (ES) m/z 441.7; HRMS: calcd for C₂₂H₂₀ClN₃O₃S+H+, 442.09866. found (ESI, [M+H]+ Obs'd), 442.0986.

### Example 100

### 2-ethyl-3-{3-[3-(methylsulfonyl)benzyl]phenyl}-8-(trifluoromethyl)imidazo[1,2-a]pyridine

In essentially the same manner as Example 63, step 2 but using 2-ethyl-8-(trifluoromethyl)imidazo[1,2-a]pyridine and 1-bromo-3-[3-(methylsulfonyl)benzyl]benzene as starting materials was prepared the title compound as a light yellow solid. MS (ESI) m/z 459.2; HRMS: calcd for C₂₄H₂₁F₃N₂O₂S+H+, 459.13486. found (ESI, [M+H]+ Obs'd), 459.1366.

### Example 101

### 2-ethyl-3-{4-[3-(methylsulfonyl)benzyl]phenyl}-8-(trifluoromethyl)imidazo[1,2-a]pyridine

In essentially the same manner as Example 63, step 2 but using 2-ethyl-8-(trifluoromethyl)imidazo[1,2-a]pyridine and 1-bromo-4-[3-(methylsulfonyl)benzyl]benzene as starting materials was prepared the title compound as a light yellow solid. MS (ESI) m/z 459.2; HRMS: calcd for C₂₄H₂₁F₃N₂O₂S+H+, 459.13486. found (ESI, [M+H]+ Obs'd), 459.1361.

### Example 102

### 2-ethyl-3-{4-[3-(methylsulfonyl)phenoxy]phenyl}-8-(trifluoromethyl)imidazo[1,2-a]pyridine

In essentially the same manner as Example 63, step 2 but using 2-ethyl-8-(trifluoromethyl)imidazo[1,2-a]pyridine and 1-(4-bromophenoxy)-3-(methylsulfonyl)benzene as starting materials was prepared the title compound as a yellow solid. MS (ESI) m/z 461.1; HRMS: calcd for C₂₃H₁₉F₃N₂O₃S+H+, 461.11412. found (ESI, [M+H]+ Obs'd), 461.1148.

### Example 103

### 8-chloro-2-ethyl-3-{4-[3-(methylsulfonyl)phenoxy]phenyl}imidazo[1,2-a]pyridine

In essentially the same manner as Example 63, step 2 but using 8-chloro-2-ethyl-imidazo[1,2-a]pyridine and 1-(4-bromophenoxy)-3-(methylsulfonyl)benzene as starting materials was prepared the title compound as a pale yellow solid. MS (ESI) m/z 427.1; HRMS: calcd for C₂₂H₁₉ClN₂O₃S+H+, 427.08777. found (ESI, [M+H]+ Obs'd), 427.0887.

### Example 104

### 2-ethyl-3-{4-[3-(methylsulfonyl)phenoxy]phenyl}imidazo[1,2-a]pyridine-8-carbonitrile

In essentially the same manner as Example 63, step 2 but using 2-ethylimidazo[1,2-a]pyridine-8-carbonitrile and 1-(4-bromophenoxy)-3-(methylsulfonyl)benzene as starting materials was prepared the title compound as a yellow solid. MS (ESI) m/z 418.1; HRMS: calcd for C₂₃H₁₉N₃O₃S+H+, 418.12199. found (ESI, [M+H]+ Obs'd), 418.1233.

### Example 105

### 2-ethyl-3-{4-[3-(ethylsulfonyl)phenoxy]phenyl}-8-(trifluoromethyl)imidazo[1,2-a]pyridine

In essentially the same manner as Example 63, step 2 but using 2-ethyl-8-(trifluoromethyl)imidazo[1,2-a]pyridine and 1-(4-bromophenoxy)-3-(ethylsulfonyl)benzene as starting materials was prepared the title compound as a yellow solid. MS (ESI) m/z 475.2; HRMS: calcd for C₂₄H₂₁F₃N₂O₃S+H+, 475.12977. found (ESI, [M+H]+ Obs'd), 475.1306.

### Example 106

### 8-chloro-2-ethyl-3-{4-[3-(ethylsulfonyl)phenoxy]phenyl}imidazo[1,2-a]pyridine

In essentially the same manner as Example 63, step 2 but using 8-chloro-2-ethyl-imidazo[1,2-a]pyridine and 1-(4-bromophenoxy)-3-(ethylsulfonyl)benzene as starting materials was prepared the title compound as an off-white solid. MS (ESI) m/z 441.1; HRMS: calcd for C₂₃H₂₁ClN₂O₃S+H+, 441.10342. found (ESI, [M+H]+ Obs'd), 441.1044.

### Example 107

### 2-ethyl-3-{4-[3-(ethylsulfonyl)phenoxy]phenyl}imidazo[1,2-a]pyridine-8-carbonitrile

In essentially the same manner as Example 63, step 2 but using 2-ethylimidazo[1,2-a]pyridine-8-carbonitrile and 1-(4-bromophenoxy)-3-(ethylsulfonyl)benzene as starting materials was prepared the title compound as a yellow solid. MS (ESI) m/z 432.1; HRMS: calcd for C₂₄H₂₁N₃O₃S+H+, 432.13764. found (ESI, [M+H]+ Obs'd), 432.1384.

### Example 108

### 2-ethyl-3-(3-{[3-(methylsulfonyl)phenoxy]methyl}phenyl)-8-(trifluoromethyl)imidazo[1,2-a]pyridine

In essentially the same manner as Example 63, step 2 but using 2-ethyl-8-(trifluoromethyl)imidazo[1,2-a]pyridine and 1-bromo-3-{[3-(methylsulfonyl)phenoxy]methyl}benzene as starting materials was prepared the title compound as an off-white solid. MS (ESI) m/z 475.1; HRMS: calcd for C₂₄H₂₁F₃N₂O₃S+H+, 475.12977. found (ESI, [M+H]+ Obs'd), 475.1309.

### Example 109

### 8-chloro-2-ethyl-3-(3-{[3-(methylsulfonyl)phenoxy]methyl}phenyl)imidazo[1,2-a]pyridine

In essentially the same manner as Example 63, step 2 but using 8-chloro-2-ethyl-imidazo[1,2-a]pyridine and 1-bromo-3-{[3-(methylsulfonyl)phenoxy]methyl}benzene as starting materials was prepared the title compound as a white solid. MS (ESI) m/z 441.1; HRMS: calcd for C₂₃H₂₁ClN₂O₃S+H+, 441.10342. found (ESI, [M+H]+ Obs'd), 441.1042.

### Example 110

### 2-ethyl-3-(3-{[3-(methylsulfonyl)phenoxy]methyl}phenyl)imidazo[1,2-a]pyridine-8-carbonitrile

In essentially the same manner as Example 63, step 2 but using 2-ethylimidazo[1,2-a]pyridine-8-carbonitrile and 1-bromo-3-{[3-(methylsulfonyl)phenoxy]methyl}benzene as starting materials was prepared the title compound as a pale yellow solid. MS (ESI) m/z 432.2; HRMS: calcd for C₂₄H₂₁N₃O₃S+H+, 432.13764. found (ESI, [M+H]+ Obs'd), 432.1390.

### Example 111

### 2-ethyl-3-(4-{[3-(methylsulfonyl)phenoxy]methyl}phenyl)-8-(trifluoromethyl)imidazo[1,2-a]pyridine

In essentially the same manner as Example 63, step 2 but using 2-ethyl-8-(trifluoromethyl)imidazo[1,2-a]pyridine and 1-bromo-4-{[3-(methylsulfonyl)phenoxy]methyl}benzene as starting materials was prepared the title compound as an off-white solid. MS (ESI) m/z 475.2; HRMS: calcd for C₂₄H₂₁F₃N₂O₃S+H+, 475.12977. found (ESI, [M+H]+ Obs'd), 475.1305.

### Example 112

### 8-chloro-2-ethyl-3-(4-{[3-(methylsulfonyl)phenoxy]methyl}phenyl)imidazo[1,2-a]pyridine

In essentially the same manner as Example 63, step 2 but using 8-chloro-2-ethyl-imidazo[1,2-a]pyridine and 1-bromo-4-{[3-(methylsulfonyl)phenoxy]methyl}benzene as starting materials was prepared the title compound as an off-white solid. MS (ESI) m/z 441.1; HRMS: calcd for C₂₃H₂₁ClN₂O₃S+H+, 441.10342. found (ESI, [M+H]+ Obs'd), 441.1044.

### Example 113

### 2-ethyl-3-(4-{[3-(methylsulfonyl)phenoxy]methyl}phenyl)imidazo[1,2-a]pyridine-8-carbonitrile

In essentially the same manner as Example 63, step 2 but using 2-ethylimidazo[1,2-a]pyridine-8-carbonitrile and 1-bromo-4-{[3-(methylsulfonyl)phenoxy]methyl}benzene as starting materials was prepared the title compound as a yellow solid. MS (ESI) m/z 432.2; HRMS: calcd for C₂₄H₂₁N₃O₃S+H+, 432.13764. found (ESI, [M+H]+ Obs'd), 432.1385.

### Example 114

### 2-isopropyl-3-(3-{3-[(methylsulfonyl)methyl]phenoxy}phenyl)-8-(trifluoromethyl)imidazo[1,2-a]pyridine

In essentially the same manner as Example 36 but using 3-[2-isopropyl-8-(trifluoromethyl)-imidazo[1,2-a]pyridin-3-yl]phenol and 1-bromo-3-[methylsulfonyl)methyl]benzene as starting materials was prepared the title compound as a fiberous white solid. MS (ESI) m/z 489.2; HRMS: calcd for C₂₅H₂₃F₃N₂O₃S+H+, 489.14542. found (ESI, [M+H]+ Obs'd), 489.1462.

### Example 115

### 2-isopropyl-3-(3-{3-[(methylsulfonyl)methyl]phenoxy}phenyl)imidazo[1,2-a]pyridine-8-carbonitrile

In essentially the same manner as Example 36 but using 3-(3-hydroxyphenyl)-2-isopropylimidazo[1,2-a]pyridine-8-carbonitrile and 1-bromo-3-[methylsulfonyl)methyl]benzene as starting materials was prepared the title compound as a pale yellow solid. MS (ESI) m/z 446.2; HRMS: calcd for C₂₅H₂₃N₃O₃S+H+, 446.15329. found (ESI, [M+H]+ Obs'd), 446.1537.

### Example 116

### 2-tert-butyl-3-(3-{3-[(methylsulfonyl)methyl]phenoxy}phenyl)imidazo[1,2-a]pyridine-8-carbonitrile

In essentially the same manner as Example 36 but using 2-tert-butyl-3-(3-hydroxyphenyl)imidazo[1,2-a]pyridine-8-carbonitrile and 1-bromo-3-[methylsulfonyl)methyl]benzene as starting materials was prepared the title compound as a yellow solid from a foam. MS (ESI) m/z 460.2; HRMS: calcd for C₂₆H₂₅N₃O₃S+H+, 460.16894. found (ESI, [M+H]+ Obs'd), 460.1694.

### Example 117

### 8-chloro-2-isopropyl-3-(3-{3-[(methylsulfonyl)methyl]phenoxy}phenyl)imidazo[1,2-a]pyridine

In essentially the same manner as Example 36 but using 3-(2-isopropyl-8-chloro-imidazo[1,2-a]pyridin-3-yl)phenol and 1-bromo-3-[methylsulfonyl)methyl]benzene as starting materials was prepared the title compound as an off-white solid from foam. MS (ESI) m/z 455.1; HRMS: calcd for C₂₄H₂₃ClN₂O₃S+H+, 455.11907. found (ESI, [M+H]+ Obs'd), 455.1197.

### Example 118

### 2-ethyl-3-(3-{3-[(methylsulfonyl)methyl]phenoxy}phenyl)imidazo[1,2-a]pyridine-8-carbonitrile

In essentially the same manner as Example 36 but using 2-ethyl-3-(3-hydroxyphenyl)imidazo[1,2-a]pyridine-8-carbonitrile and 1-bromo-3-[methylsulfonyl)methyl]benzene as starting materials was prepared the title compound as a pale yellow solid. MS (ESI) m/z 432.2; HRMS: calcd for C₂₄H₂₁N₃O₃S+H+, 432.13764. found (ESI, [M+H]+ Obs'd), 432.1381.

### Example 119

### 2-(1-methylethyl)-3-{4-[3-(methylsulfonyl)phenoxy]phenyl}-8-(trifluoromethyl)imidazo[1,2-a]pyridine

In essentially the same manner as Example 75, step 2 but using 2-isopropyl-8-(trifluoromethyl)imidazo[1,2-a]pyridine and 1-(4-bromophenoxy)-3-(methylsulfonyl)benzene as starting materials was prepared the title compound as a yellow solid. MS (ESI) m/z 475.2; HRMS: calcd for C₂₄H₂₁F₃N₂O₃S+H+, 475.12977. found (ESI, [M+H]+ Obs'd), 475.1299.

### Example 120

### 3-{4-[3-(ethylsulfonyl)phenoxy]phenyl}-2-(1-methylethyl)-8-(trifluoromethyl)imidazo[1,2-a]pyridine

In essentially the same manner as Example 75, step 2 but using 2-isopropyl-8-(trifluoromethyl)imidazo[1,2-a]pyridine and 1-(4-bromophenoxy)-3-(ethylsulfonyl)benzene as starting materials was prepared the title compound as a yellow solid. MS (ESI) m/z 489.2; HRMS: calcd for C₂₅H₂₃F₃N₂O₃S+H+, 489.14542. found (ESI, [M+H]+ Obs'd), 489.1458.

### Example 121

### 8-chloro-2-(1-methylethyl)-3-{4-[3-(methylsulfonyl)phenoxy]phenyl}imidazo[1,2-a]pyridine

In essentially the same manner as Example 75, step 2 but using 2-isopropyl-8-chloroimidazo[1,2-a]pyridine and 1-(4-bromophenoxy)-3-(methylsulfonyl)benzene as starting materials was prepared the title compound as an off white solid. MS (ESI) m/z 441.1; HRMS: calcd for C₂₃H₂₁ClN₂O₃S+H+, 441.10342. found (ESI, [M+H]+ Obs'd), 441.1036.

### Example 122

### 8-chloro-3-{4-[3-(ethylsulfonyl)phenoxy]phenyl}-2-(1-methylethyl)imidazo[1,2-a]pyridine

In essentially the same manner as Example 75, step 2 but using 2-isopropyl-8-chloroimidazo[1,2-a]pyridine and 1-(4-bromophenoxy)-3-(ethylsulfonyl)benzene as starting materials was prepared the title compound as a yellow solid. MS (ESI) m/z 455.1; HRMS: calcd for C₂₄H₂₃ClN₂O₃S+H+, 455.11907. found (ESI, [M+H]+ Obs'd), 455.1196.

### Example 123

### 2-(1-methylethyl)-3-{4-[3-(methylsulfonyl)phenoxy]phenyl}imidazo[1,2-a]pyridine-8-carbonitrile

In essentially the same manner as Example 75, step 2 but using 2-isopropylimidazo[1,2-a]pyridine-8-carbonitrile and 1-(4-bromophenoxy)-3-(methylsulfonyl)benzene as starting materials was prepared the title compound as an off-white solid. MS (ESI) m/z 432.2; HRMS: calcd for C₂₄H₂₁N₃O₃S+H+, 432.13764. found (ESI, [M+H]+ Obs'd), 432.1377.

### Example 124

### 3-{4-[3-(ethylsulfonyl)phenoxy]phenyl}-2-(1-methylethyl)imidazo[1,2-a]pyridine-8-carbonitrile

In essentially the same manner as Example 75, step 2 but using 2-isopropylimidazo[1,2-a]pyridine-8-carbonitrile and 1-(4-bromophenoxy)-3-(ethylsulfonyl)benzene as starting materials was prepared the title compound as a yellow solid. MS (ESI) m/z 446.2; HRMS: calcd for C₂₅H₂₃N₃O₃S+H+, 446.15329. found (ESI, [M+H]+ Obs'd), 446.1534.

### Example 125

### 3-{4-[3-(methylsulfonyl)phenoxy]phenyl}-2,8-bis(trifluoromethyl)imidazo[1,2-a]pyridine

In essentially the same manner as Example 75, step 2 but using 2,8-bis(trifluoromethyl)imidazo[1,2-a]pyridine and 1-(4-bromophenoxy)-3-(methylsulfonyl)benzene as starting materials was prepared the title compound as a yellow solid. MS (ESI) m/z 501.1; HRMS: calcd for C₂₂H₁₄F₆N₂O₃S+H+, 501.07021. found (ESI, [M+H]+ Obs'd), 501.0707.

### Example 126

### 2-(2-methoxyphenyl)-3-{4-[3-(methylsulfonyl)phenoxy]phenyl}-8-(trifluoromethyl)imidazo[1,2-a]pyridine

In essentially the same manner as Example 75, step 2 but using 2-(2-methoxyphenyl)-8-(trifluoromethyl)imidazo[1,2-a]pyridine and 1-(4-bromophenoxy)-3-(methylsulfonyl)benzene as starting materials was prepared the title compound as a yellow solid. MS (ESI) m/z 539.2; HRMS: calcd for C₂₈H₂₁F₃N₂O₄S+H+, 539.12469. found (ESI, [M+H]+ Obs'd), 539.1258.

### Example 127

### 3-{4-[3-(methylsulfonyl)phenoxy]phenyl}-2-thiophen-3-yl-8-(trifluoromethyl)imidazo[1,2-a]pyridine

In essentially the same manner as Example 75, step 2 but using 2-(thiophen-3-yl)-8-(trifluoromethyl)imidazo[1,2-a]pyridine and 1-(4-bromophenoxy)-3-(methylsulfonyl)benzene as starting materials was prepared the title compound as a yellow solid. MS (ESI) m/z 515.1; HRMS: calcd for C₂₅H₁₇F₃N₂O₃S₂+H+, 515.07054. found (ESI, [M+H]+ Obs'd), 515.0706.

### Example 128

### 2-(butoxymethyl)-3-{4-[3-(methylsulfonyl)phenoxy]phenyl}-8-(trifluoromethyl)imidazo[1,2-a]pyridine

In essentially the same manner as Example 75, step 2 but using 2-(butoxymethyl)-8-(trifluoromethyl)imidazo[1,2-a]pyridine and 1-(4-bromophenoxy)-3-(methylsulfonyl)benzene as starting materials was prepared the title compound as a yellow glassy solid. MS (ESI) m/z 519.2; HRMS: calcd for C₂₆H₂₅F₃N₂O₄S+H+, 519.15599. found (ESI, [M+H]+ Obs'd), 519.1575.

### Example 129

### 2-(1H-imidazol-1-ylmethyl)-3-{3-[3-(methylsulfonyl)phenoxy]phenyl}-8-(trifluoromethyl)imidazo[1,2-a]pyridine

### Step 1: 2-(bromomethyl)-3-{3-[3-(methylsulfonyl)phenoxy]phenyl}-8-(trifluoromethyl)imidazo[1,2-a]pyridine

A mixture of 2-methyl-3-(3-(3-(methylsulfonyl)phenoxy)phenyl)-8-(trifluoromethyl)imidazo[1,2-a]pyridine (1.65 g, 3.70 mmol), N-bromosuccinimide (0.724 g, 4.07 mmol) and α,α-azobisisobutyronitrile (0.061 g, 0.370 mmol) in acetonitrile (20 mL) was refluxed for 3 h. The solvent was removed and the resulting material was partitioned between dichloromethane and water. The layers were separated and the aqueous layer was further extracted with dichloromethane. The combined dichloromethane layers were concentrated. Purification via column chromatography using a 0:100 to 55:45 E:H gradient afforded the title compound as a yellow foam solid (1.54 g, 79%).

### Step 2: 2-(1H-imidazol-1-ylmethyl)-3-{3-[3-(methylsulfonyl)phenoxy]phenyl}-8-(trifluoromethyl)imidazo[1,2-a]pyridine

A mixture of 2-(bromomethyl)-3-{3-[3-(methylsulfonyl)phenoxy]phenyl}-8-(trifluoromethyl)imidazo[1,2-a]pyridine (0.100 g, 0.190 mmol), 1H-imidazole (0.039 g, 0.571 mmol) and potassium carbonate (0.053 g, 0.381 mmol) in DMSO (3 mL) was stirred for 1 h. Water was added, then the mixture was extracted with dichloromethane. The combined organics were washed with water then concentrated. Purification via column chromatography using a 0:100 to 5:95 MeOH:CH₂Cl₂ gradient yielded the title compounds as a white foam solid (0.058 g, 60%). MS (ESI) m/z 513.1; HRMS: calcd for C₂₅H₁₉F₃N₄O₃S+H+, 513.12027. found (ESI, [M+H]+ Obs'd), 513.1205.

### Example 130

### 2-[(2-methyl-1H-imidazol-1-yl)methyl]-3-{3-[3-(methylsulfonyl)phenoxy]phenyl}-8-(trifluoromethyl)imidazo[1,2-a]pyridine

In essentially the same manner as Example 129 but using 2-methyl-1H-imidazole in place of 1H-imidazole was prepared the title compound as a yellow foam solid. MS (ESI) m/z 527.2; HRMS: calcd for C₂₆H₂₁F₃N₄O₃S+H+, 527.13592. found (ESI, [M+H]+ Obs'd), 527.1363.

### Example 131

### 3-{3-[3-(methylsulfonyl)phenoxy]phenyl}-2-(piperidin-1-ylmethyl)-8-(trifluoromethyl)imidazo[1,2-a]pyridine

A mixture of 2-(bromomethyl)-3-{3-[3-(methylsulfonyl)phenoxy]phenyl}-8-(trifluoromethyl)imidazo[1,2-a]pyridine (0.100 g, 0.190 mmol) and piperidine (0.081 g, 0.952 mmol) in ethanol (3 mL) was stirred overnight. The mixture was concentrated. To the resulting material was added dichloromethane and water. The solution was made basic by addition of solid potassium carbonate. The layers were separated. The aqueous layer was extracted once more with dichloromethane and the combined organic layers were concentrated. Purification via column chromatography using an ISCO amine column with a 0:100 to 60:40 E:H gradient afforded the title compound as an off-white solid (0.069 g, 68%). MS (ESI) m/z 530.2; HRMS: calcd for C₂₇H₂₆F₃N₃O₃S+H+, 530.17197. found (ESI, [M+H]+ Obs'd), 530.1721.

### Example 132

### 2-[(4-methylpiperazin-1-yl)methyl]-3-{3-[3-(methylsulfonyl)phenoxy]phenyl}-8-(trifluoromethyl)imidazo[1,2-a]pyridine

In essentially the same manner as Example 131 but using 1-methylpiperazine in place of piperidine afforded the title compound as an off-white solid. MS (ESI) m/z 545.2; HRMS: calcd for C₂₇H₂₇F₃N₄O₃S+H+, 545.18287. found (ESI, [M+H]+ Obs'd), 545.1830.

### Example 133

### 3-{3-[3-(methylsulfonyl)phenoxy]phenyl}-2-(1,3-thiazolidin-3-ylmethyl)-8-(trifluoromethyl)imidazo[1,2-a]pyridine

In essentially the same manner as Example 131 but using thiazolidine in place of piperidine afforded the title compound as an off-white solid. MS (ESI) m/z 534.1; HRMS: calcd for C₂₅H₂₂F₃N₃O₃S₂+H+, 534.11274. found (ESI, [M+H]+ Obs'd), 534.1132.

### Example 134

### N-methyl-1-[3-{3-[3-(methylsulfonyl)phenoxy]phenyl}-8-(trifluoromethyl)imidazo[1,2-a]pyridin-2-yl]methanamine

In essentially the same manner as Example 131 but using methylamine in place of piperidine afforded the title compound as an off-white solid. MS (ESI) m/z 476.2.

### Example 135

### N-{[3-{3-[3-(methylsulfonyl)phenoxy]phenyl}-8-(trifluoromethyl)imidazo[1,2-a]pyridin-2-yl]methyl}ethanamine

In essentially the same manner as Example 131 but using ethylamine in place of piperidine afforded the title compound as a white solid. MS (ESI) m/z 490.2; HRMS: calcd for C₂₄H₂₂F₃N₃O₃S+H+, 490.14067. found (ESI, [M+H]+ Obs'd), 490.1410.

### Example 136

N-{[3-{3-[3-(methylsulfonyl)phenoxy]phenyl}-8-(trifluoromethyl)imidazo[1,2-a]pyridin-2-yl]methyl}propan-2-amine

In essentially the same manner as Example 131 but using isopropylamine in place of piperidine afforded the title compound as an off-white solid. MS (ESI) m/z 504.2; HRMS: calcd for C₂₅H₂₄F₃N₃O₃S+H+, 504.15632. found (ESI, [M+H]+ Obs'd), 504.1565.

### Example 137

### N,N-dimethyl-1-[3-{3-[3-(methylsulfonyl)phenoxy]phenyl}-8-(trifluoromethyl)imidazo[1,2-a]pyridin-2-yl]methanamine

In essentially the same manner as Example 131 but using dimethylamine in place of piperidine afforded the title compound as a yellow solid. MS (ESI) m/z 490.2; HRMS: calcd for C₂₄H₂₂F₃N₃O₃S+H+, 490.14067. found (ESI, [M+H]+ Obs'd), 490.1407.

### Example 138

### N-ethyl-N-{[3-{3-[3-(methylsulfonyl)phenoxy]phenyl}-8-(trifluoromethyl)imidazo[1,2-a]pyridin-2-yl]methyl}ethanamine

In essentially the same manner as Example 131 but using diethylamine in place of piperidine afforded the title compound as a yellow glassy solid. MS (ESI) m/z 518.2; HRMS: calcd for C₂₆H₂₆F₃N₃O₃S+H+, 518.17197. found (ESI, [M+H]+ Obs'd), 518.1722.

### Example 139

### 3-{3-[3-(methylsulfonyl)phenoxy]phenyl}-2-(pyrolidin-1-ylmethyl)-8-(trifluoromethyl)imidazo[1,2-a]pyridine

In essentially the same manner as Example 131 but using pyrrolidine in place of piperidine afforded the title compound as a tan solid. MS (ESI) m/z 516.2; HRMS: calcd for C₂₆H₂₄F₃N₃O₃S+H+, 516.15632. found (ESI, [M+H]+ Obs'd), 516.1571.

### Example 140

### 4-[(3-{3-[2-ethyl-8-(trifluoromethyl)imidazo[1,2-a]pyridin-3-yl]phenoxy}phenyl)sulfonyl]butanenitrile

The title compound was prepared in a manner similar to that described in Example 25 except using 3-(2-ethyl-8-(trifluoromethyl)imidazo[1,2-a]pyridin-3-yl)phenol in place of 3-(2-isopropyl-8-(trifluoromethyl)imidazo[1,2-a]pyridin-3-yl)phenol and 4-[(3-bromophenyl)sulfonyl]butanenitrile in place of 1-bromo-3-(methylsulfonyl)benzene. It was isolated as a white powder from a foam. MS (ESI) m/z 514.1; HRMS: calcd for C₂₆H₂₂F₃N₃O₃S+H⁺, 514.14067. found (ESI, [M+H]⁺ Obs'd), 514.1407.

### Example 141

### 4-{3-[2-ethyl-8-(trifluoromethyl)imidazo[1,2-a]pyridin-3-yl]phenoxy}-2-(methylsulfonyl)benzonitrile

The title compound was prepared in a manner similar to that described in Example 9 except using 3-{2-ethyl-8-(trifluoromethyl)imidazo[1,2-a]pyridin-3-yl}phenol in place of 3-{(2-methyl-8-(trifluoromethyl)imidazo[1,2-a]pyridin-3-yl}phenol and 4-fluoro-2-(methylsulfonyl)benzonitrile in place of 1-fluoro-3-(methylsulfonyl)benzene. The reaction temperature was 50° C. The compound was isolated as a white solid. MS (ESI) m/z 486.1; HRMS: calcd for C₂₄H₁₈F₃N₃O₃S+H⁺, 486.10937. found (ESI, [M+H]⁺ Obs'd), 486.1100.

### Example 142

### 2-(methylsulfonyl)-4-{3-[2-methyl-8-(trifluoromethyl)imidazo[1,2-a]pyridin-3-yl]phenoxy}benzonitrile

The title compound was prepared in a manner similar to that described in Example 9 except using 4-fluoro-2-(methylsulfonyl)benzonitrile in place of 1-fluoro-3-(methylsulfonyl)benzene. The reaction temperature was 50° C. The compound was isolated as a white solid. MS (ESI) m/z 472.1; HRMS: calcd for C₂₃H₁₆F₃N₃O₃S+H⁺, 472.09372. found (ESI, [M+H]⁺ Obs'd), 472.0942.

The structures of the title compounds of Examples 1-142 are set forth below.

| Example | Chemical Structure |
|---|---|
| 1 | 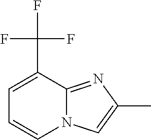 |
| 2 | 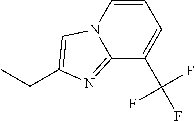 |
| 3 | 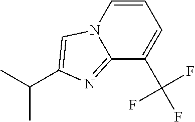 |
| 4 | 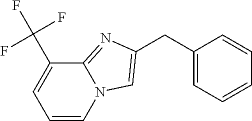 |
| 5 | 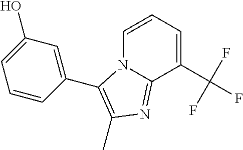 |
| 6 | 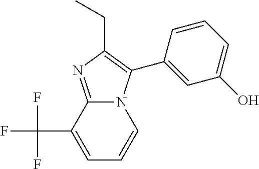 |
| 7 | 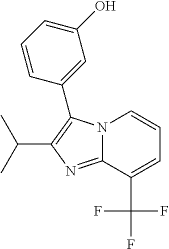 |
| 8 | 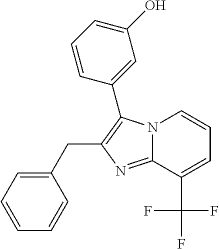 |
| 9 | 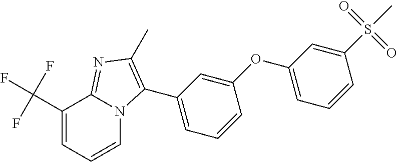 |
| 10 | 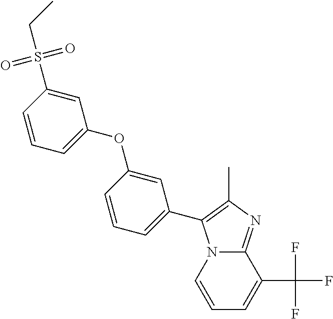 |
| 11 | 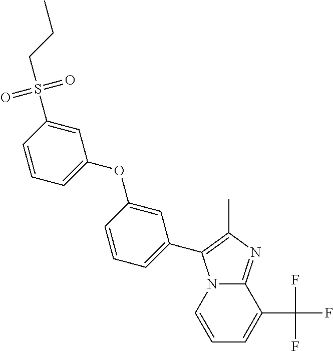 |
| 12 | 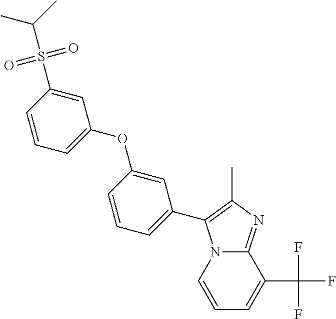 |
| 13 | 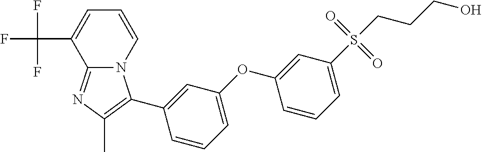 |
| 14 | 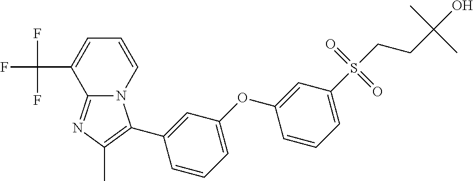 |
| 15 | 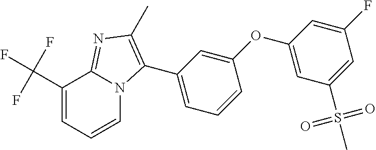 |
| 16 | 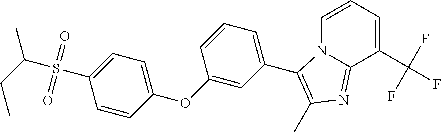 |
| 17 | 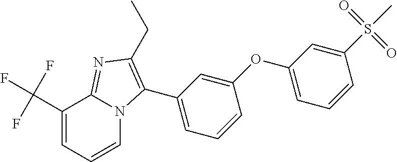 |
| 18 | 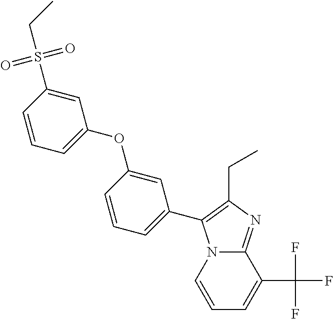 |
| 19 | 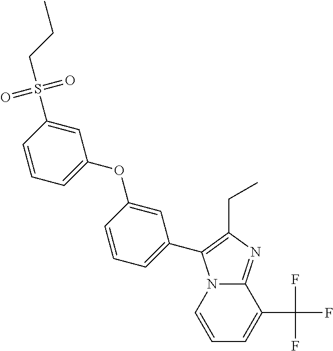 |
| 20 | 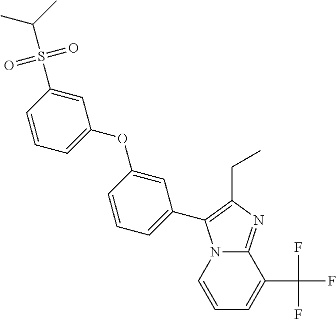 |
| 21 | 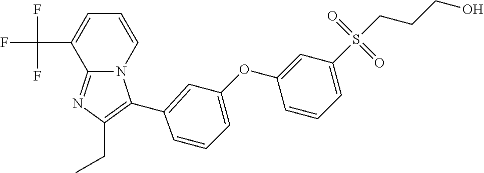 |
| 22 | 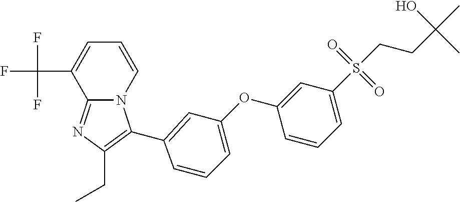 |
| 23 | 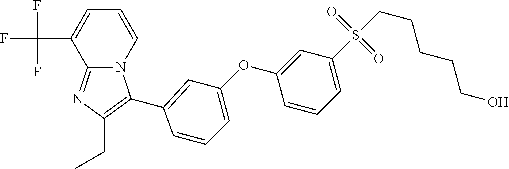 |
| 24 | 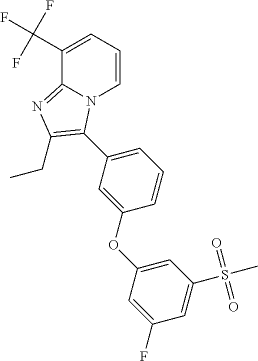 |
| 25 | 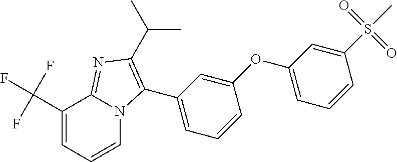 |
| 26 | 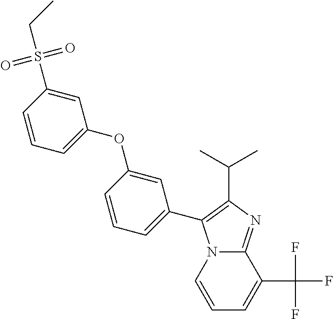 |
| 27 | 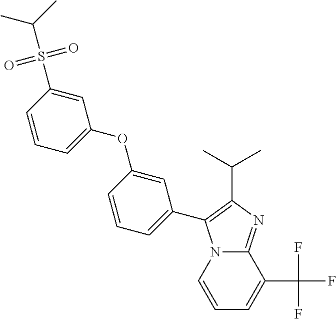 |
| 28 | 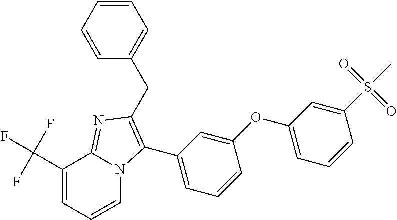 |
| 29 | 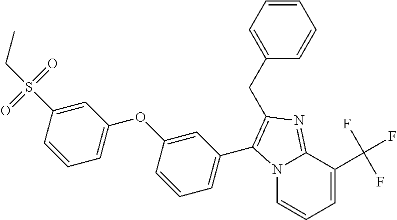 |
| 30 | 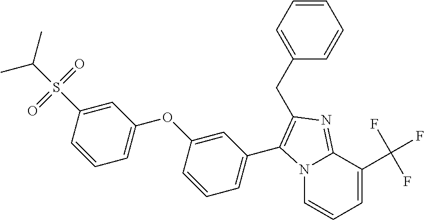 |
| 31 | 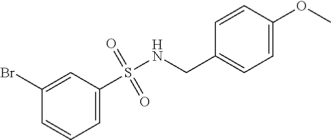 |
| 32 | 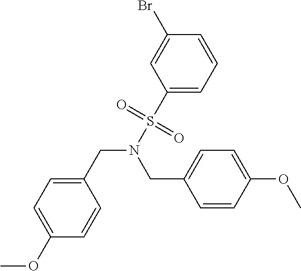 |
| 33 | 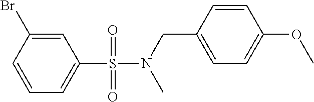 |
| 34 | 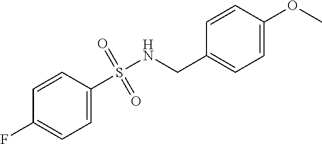 |
| 35 | 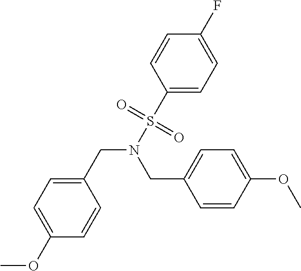 |
| 36 | 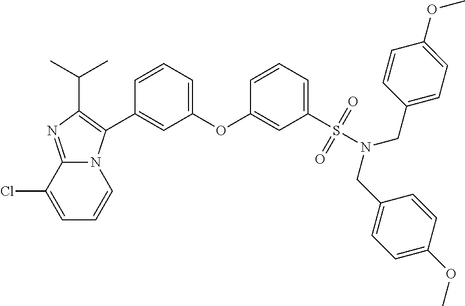 |
| 37 | 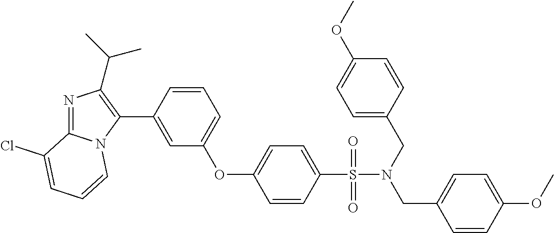 |
| 38 | 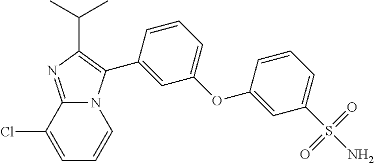 |
| 39 | 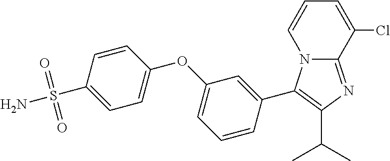 |
| 40 | 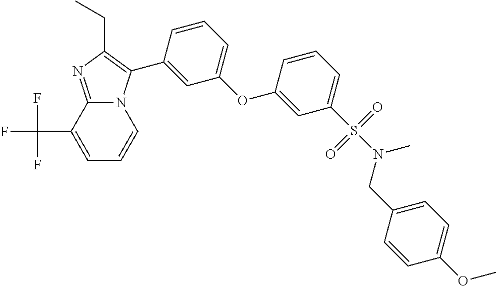 |
| 41 | 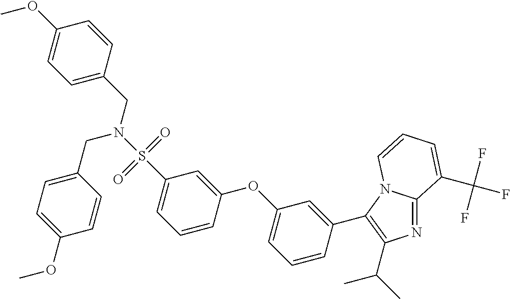 |
| 42 | 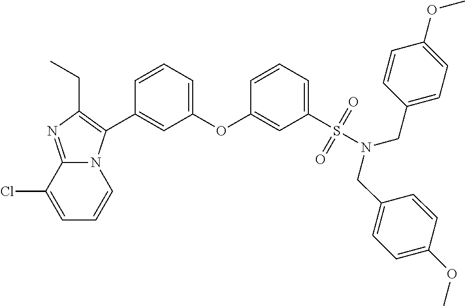 |
| 43 | 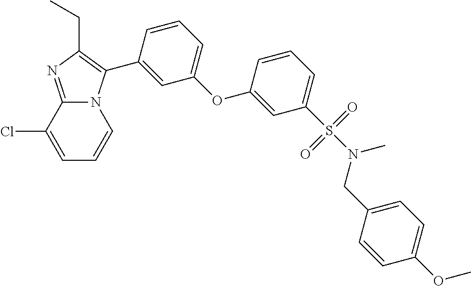 |
| 44 | 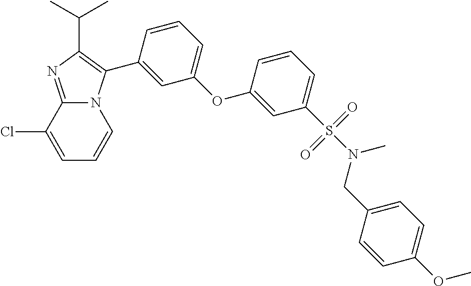 |
| 45 | 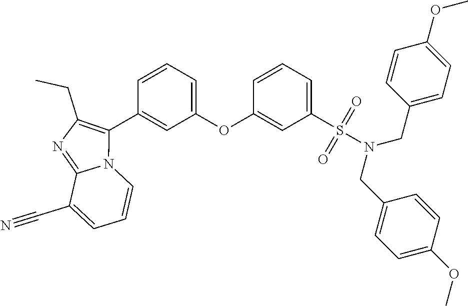 |
| 46 | 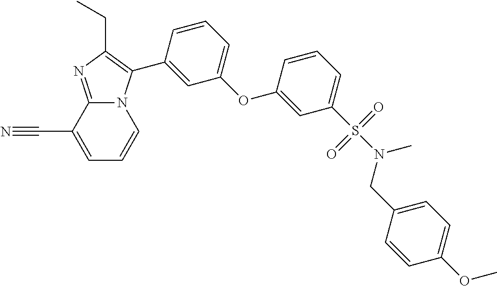 |
| 47 | 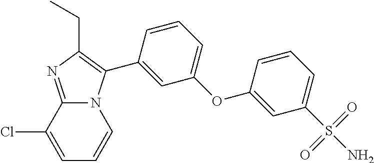 |
| 48 | 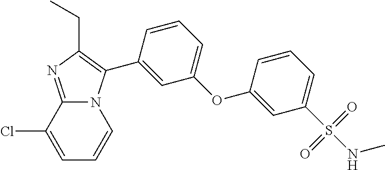 |
| 49 | 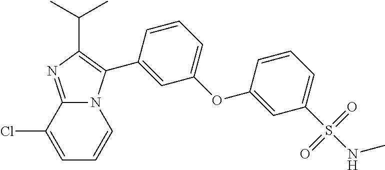 |
| 50 | 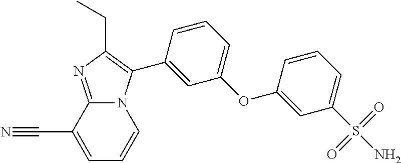 |
| 51 | 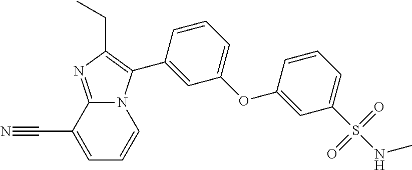 |
| 52 | 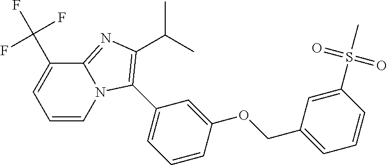 |
| 53 | 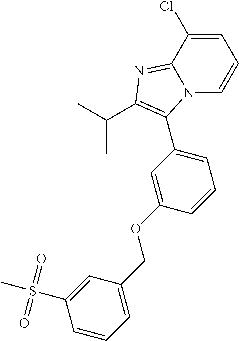 |
| 54 | 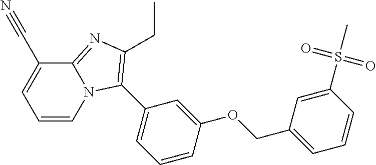 |
| 55 | 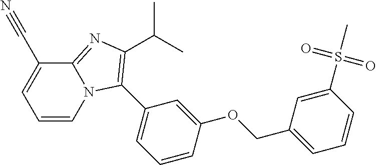 |
| 56 | 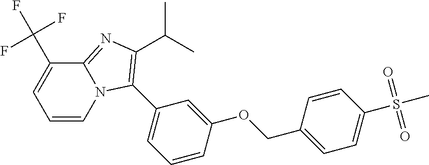 |
| 57 | 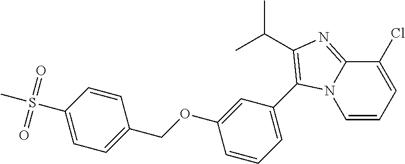 |
| 58 | 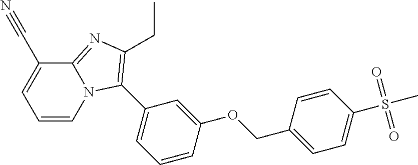 |
| 59 | 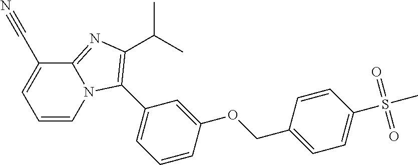 |
| 60 | 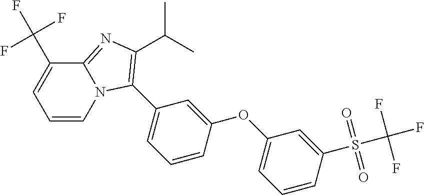 |
| 61 | 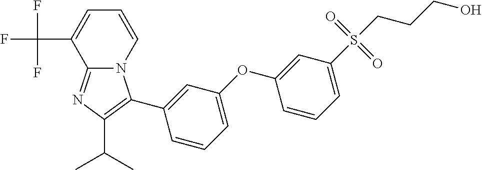 |
| 62 | 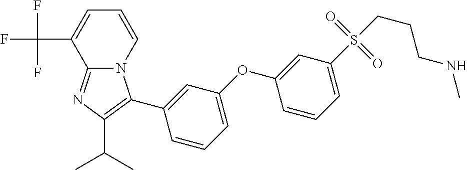 |
| 63 | 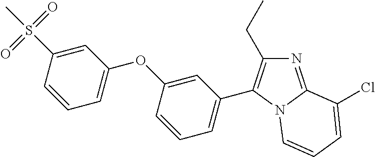 |
| 64 | 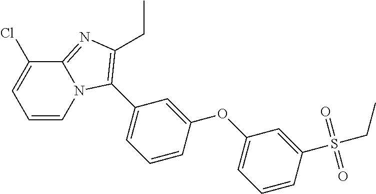 |
| 65 | 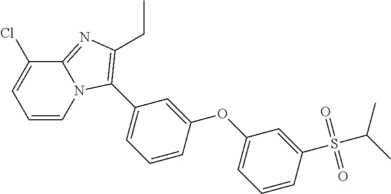 |
| 66 | 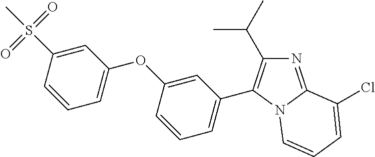 |
| 67 | 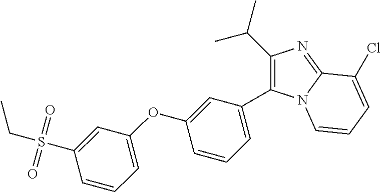 |
| 68 | 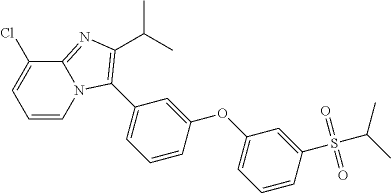 |
| 69 | 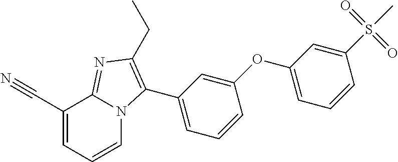 |
| 70 | 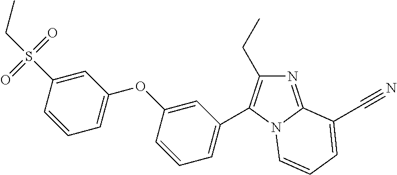 |
| 71 | 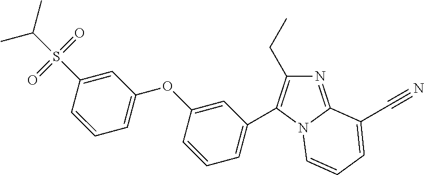 |
| 72 | 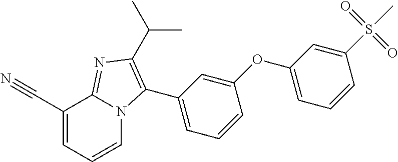 |
| 73 | 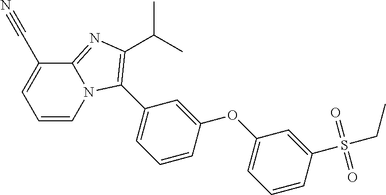 |
| 74 | 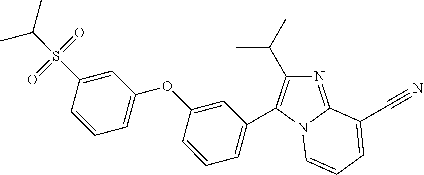 |
| 75 | 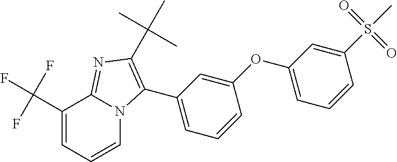 |
| 76 | 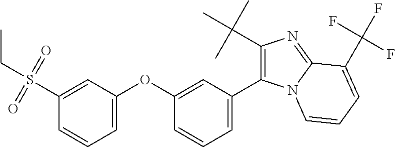 |
| 77 | 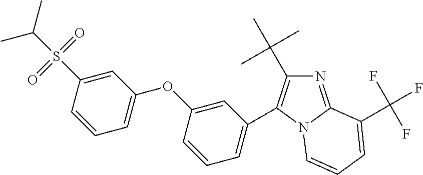 |
| 78 | 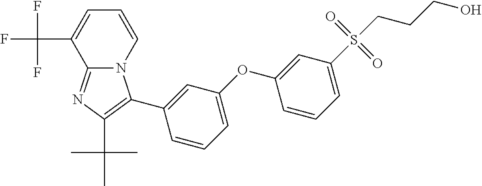 |
| 79 | 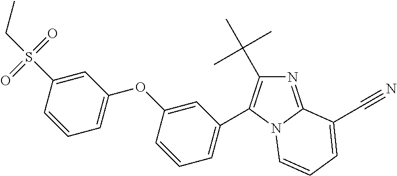 |
| 80 | 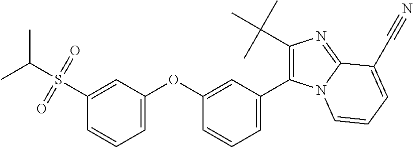 |
| 81 | 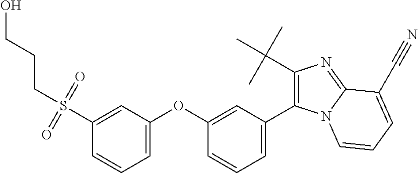 |
| 82 | 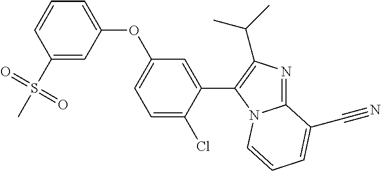 |
| 83 | 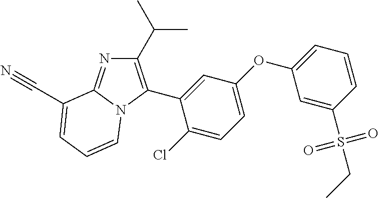 |
| 84 | 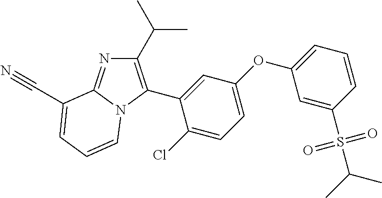 |
| 85 | 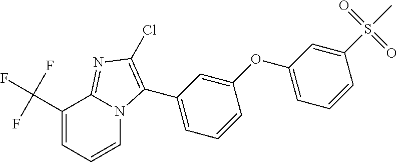 |
| 86 | 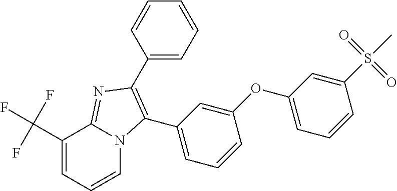 |
| 87 | 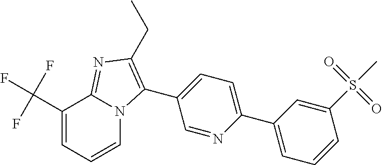 |
| 88 | 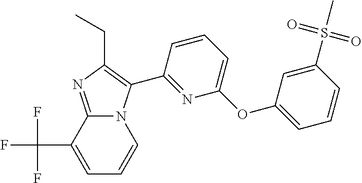 |
| 89 | 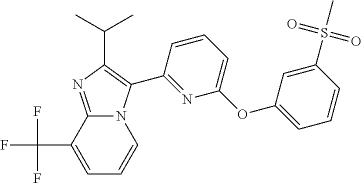 |
| 90 | 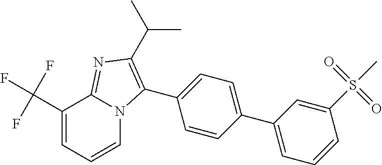 |
| 91 | 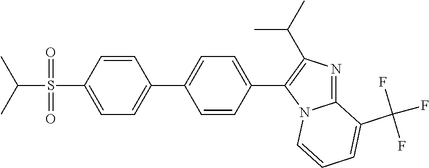 |
| 92 | 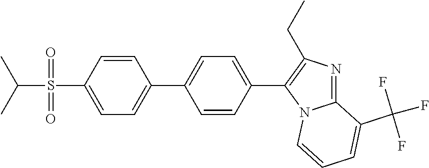 |
| 93 | 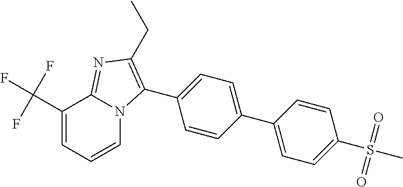 |
| 94 | 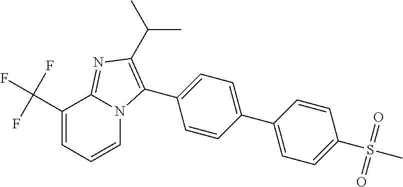 |
| 95 | 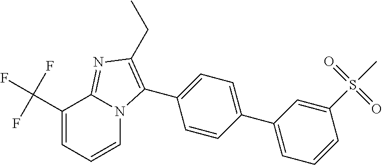 |
| 96 | 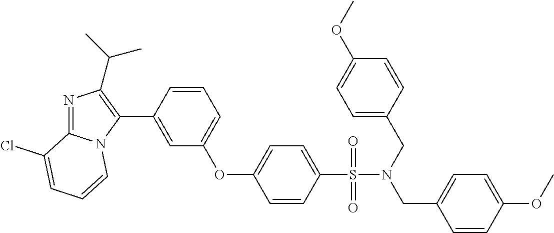 |
| 97 | 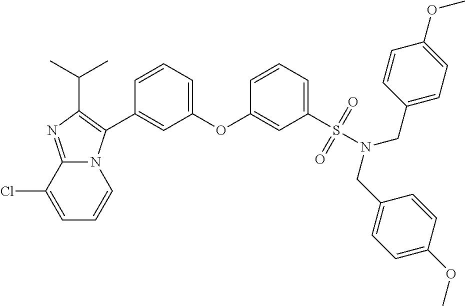 |
| 98 | 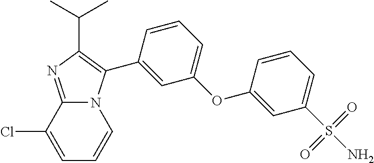 |
| 99 | 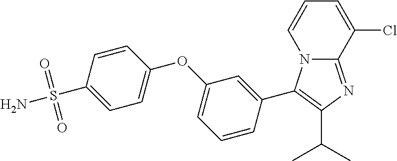 |
| 100 | 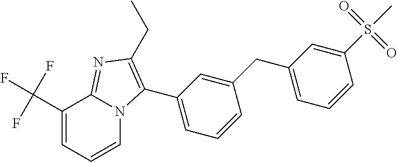 |
| 101 | 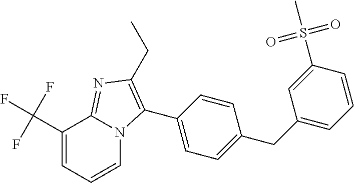 |
| 102 | 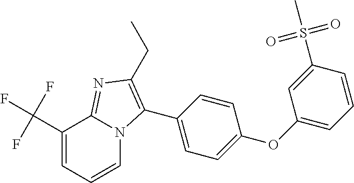 |
| 103 | 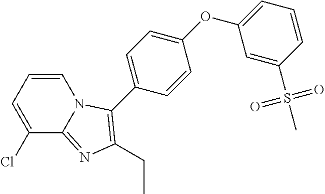 |
| 104 | 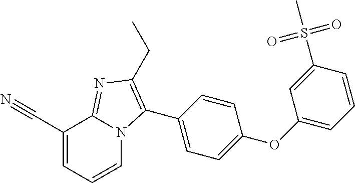 |
| 105 | 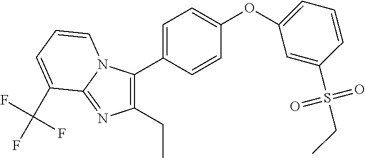 |
| 106 | 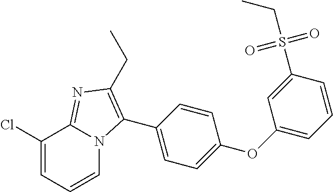 |
| 107 | 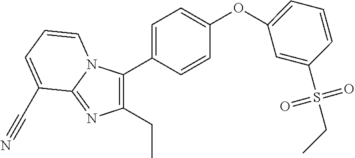 |
| 108 | 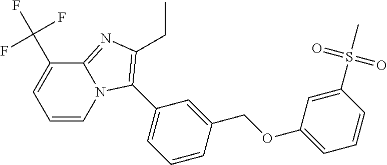 |
| 109 | 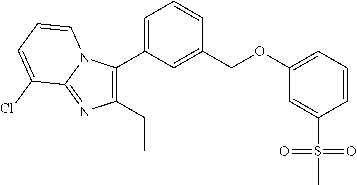 |
| 110 | 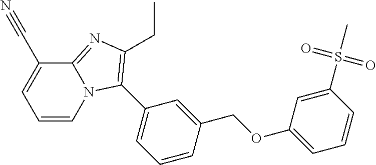 |
| 111 | 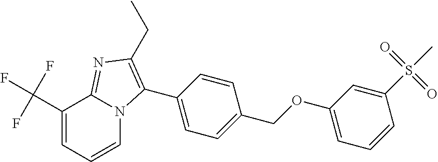 |
| 112 | 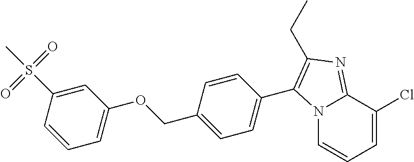 |
| 113 | 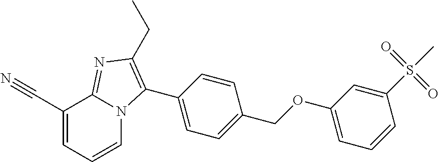 |
| 114 | 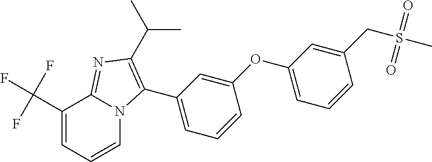 |
| 115 | 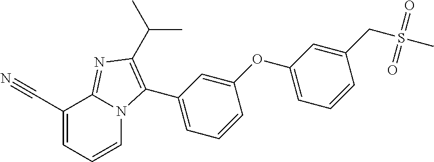 |
| 116 | 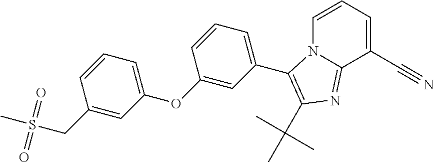 |
| 117 | 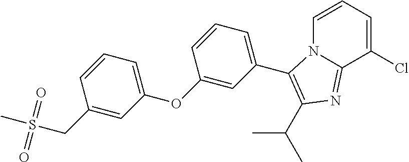 |
| 118 | 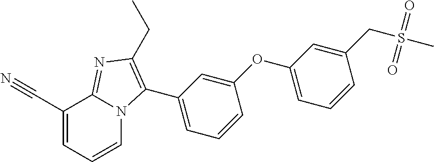 |
| 119 | 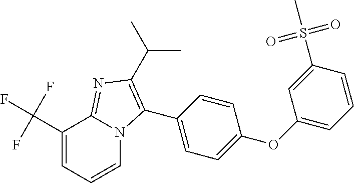 |
| 120 | 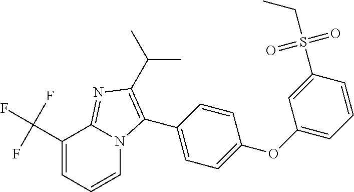 |
| 121 | 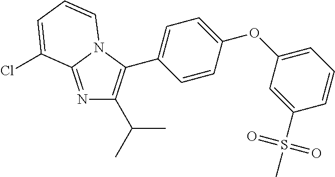 |
| 122 | 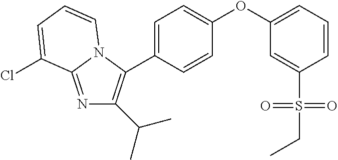 |
| 123 | 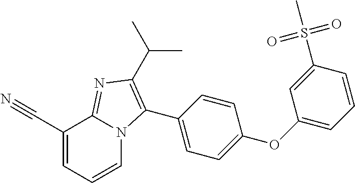 |
| 124 | 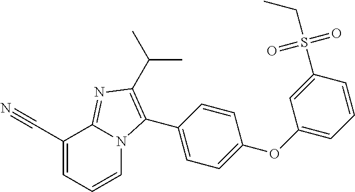 |
| 125 | 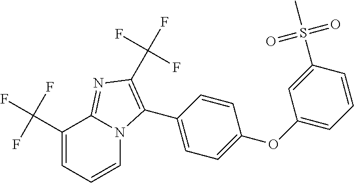 |
| 126 | 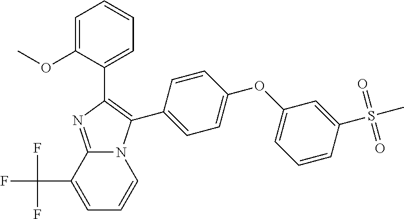 |
| 127 | 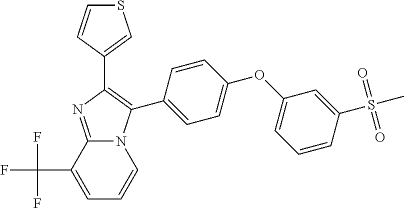 |
| 128 | 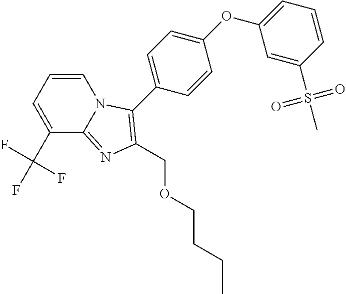 |
| 129 | 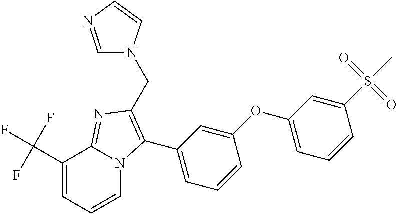 |
| 130 | 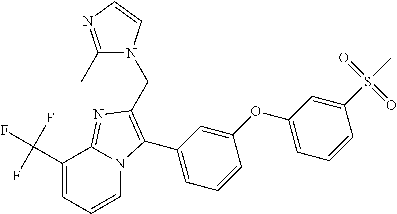 |
| 131 | 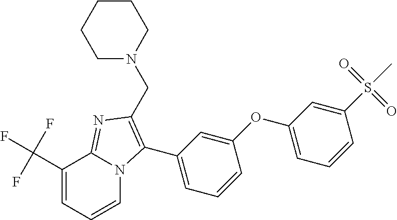 |
| 132 | 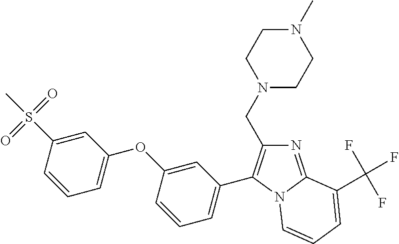 |
| 133 | 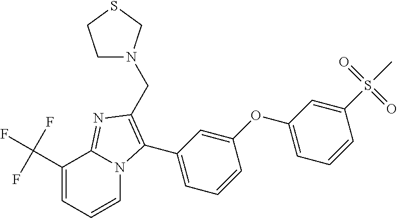 |
| 134 | 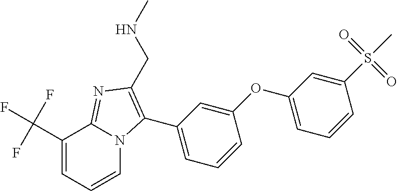 |
| 135 | 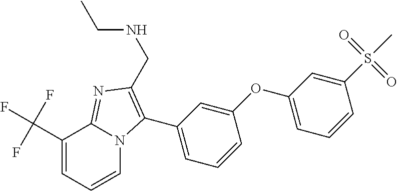 |
| 136 | 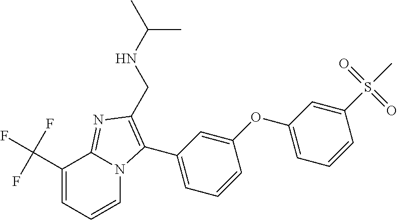 |
| 137 | 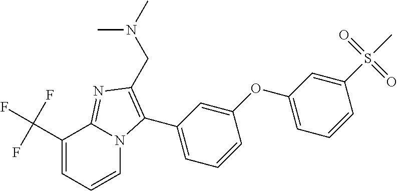 |
| 138 | 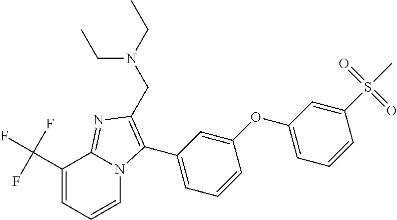 |
| 139 | 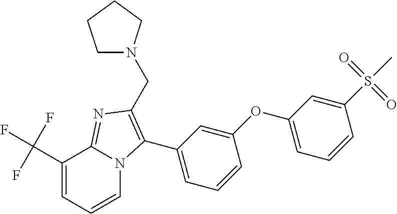 |
| 140 | 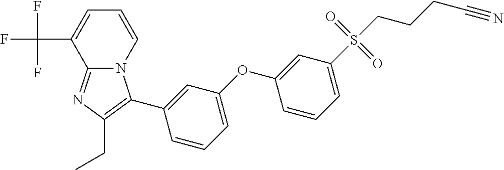 |
| 141 | 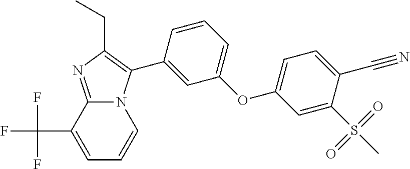 |
| 142 | 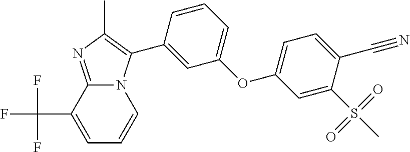 |

### Example 143

### Biological Testing

Representative compounds of this invention were evaluated in conventional pharmacological test procedures which measured their affinity to bind to LXR and to upregulate the gene ABCA1, which causes cholesterol efflux from atherogenic cells, such as macrophages.

LXR activation can be critical for maintaining cholesterol homeostasis, but its coincident regulation of fatty acid metabolism may lead to increased serum and hepatic triglyceride levels. Selective LXR modulators that activate cholesterol efflux with minimal impact on SREBP-1c expression and triglyceride synthesis in liver would be expected to reduce atherosclerotic risk with an improved therapeutic index and minimize the potential for deleterious effects on metabolic balance.

The test procedures performed, and results obtained, are briefly described in the following sections:

I. Ligand-Binding Test Procedure for Human LXRβ

II. Ligand-Binding Test Procedure for Human LXRα

III. Quantitative Analysis of ABCA1 Gene Regulation in THP-1 Cells

IV. Results

### I. Ligand-Binding Test Procedure for Human LXRβ.

Ligand-binding to the human LXRβ was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

### Tracer: ³H T0901317

Receptor source: E. coli extract from cells expressing biotinylated hLXRβ. Extract was made in a similar buffer as above, but with 50 mM TRIS.

### Day 1

Washed streptavidin and coated flash plates with wash buffer.
Diluted receptor extract to give Bmax ˜4000 cpm and add to the wells.
Wrapped the plates in aluminum foil and stored them at +4° C. over night.

### Day 2

Made a dilution series in DMSO of the test ligands.
Made a 5 nM solution of the radioactive tracer in buffer.
Mixed 250 μl diluted tracer with 5 μl of the test ligand from each concentration of the dilution series.
Washed the receptor-coated flash plates.
Added 200 μl per well of the ligand/radiolabel mixture to the receptor-coated flash plates.
Wrapped the plates in aluminum foil and incubate at +4° C. over night.

### Day 3

Aspirated wells, and wash the flashed plates. Sealed the plate.
Measured the remaining radioactivity in the plate.

### II. Ligand-Binding Test Procedure for Human LXRα.

Ligand-binding to the human LXRα was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

### Tracer: ³H T0901317

Receptor source: E. coli extract from cells expressing biotinylated hLXRα. Extract was made in a similar buffer as above, but with 50 mM TRIS.

### Day 1

Washed streptavidin and coated flash plates with wash buffer.
Diluted receptor extract to give Bmax ˜4000 cpm and add to the wells.
Wrapped the plates in aluminum foil and stored them at +4° C. over night.

### Day 2

Made a dilution series in DMSO of the test ligands.
Made a 5 nM solution of the radioactive tracer in buffer.
Mixed 250 μl diluted tracer with 5 μl of the test ligand from each concentration of the dilution series.
Washed the receptor-coated flash plates.
Added 200 μl per well of the ligand/radiolabel mixture to the receptor-coated flash plates.
Wrapped the plates in aluminum foil and incubate at +4° C. over night.

### Day 3

Aspirated wells, and wash the flashed plates. Sealed the plate.
Measured the remaining radioactivity in the plate.

### III. Quantitative Analysis of ABCA1 Gene Regulation in THP-1 Cells.

The compounds of formula (I) effect on the regulation of the ABCA1 gene was evaluated using the following procedure.

### Materials and Methods

Cell culture: The THP-1 monocytic cell line (ATCC #TIB-202) was obtained from American Type Culture Collection (Manassas, Va.) and cultured in RPMI 1640 medium (Gibco, Carlsbad, Calif.) containing 10% FBS, 2 mM L-glutamine, and 55 uM beta-Mercaptoethanol (BME). Cells were plated in 96-well format at a density of 7.5×10⁴ in complete medium containing 50-100 ng/ml phorbal 12,13-dibutyrate (Sigma, St. Louis, Mo.) for three days to induce differentiation into adherent macrophages. Differentiated THP-1 cells were treated with test compounds or ligands dissolved in DMSO (Sigma, D-8779) in culture medium lacking phorbal ester. Final concentrations of DMSO did not exceed 0.3% of the media volume. Dose response effects were measured in duplicate, in the range of 0.001 to 30 micromolar concentrations and treated cells were incubated for an additional 18 hrs prior to RNA isolation. Unstimulated cells treated with vehicle were included as negative controls on each plate. An LXR agonist reference, N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838), was dosed at 1.0 uM and served as a positive control. In antagonist mode, the compound under study is analyzed in the presence of 150 nM GW3965, trifluoromethyl-benzyl)-(2,2-diphenyl-ethyl)-amino]-propoxy]-phenyl)-acetic acid (Collins, J. L., J. Med. Chem. (2000), 45:1963-1966.). Results of antagonist analysis are expressed as % antagonism and IC50 (in μM).
RNA isolation and quantitation: Total cellular RNA was isolated from treated cells cultured in 96-well plates using PrepStation 6100 (Applied Biosystems, Foster City, Calif.), according to the manufacturer's recommendations. RNA was resuspended in ribonuclease-free water and stored at −70° C. prior to analysis. RNA concentrations were quantitated with RiboGreen test procedure, #R-11490 (Molecular Probes, Eugene, Oreg.).
Gene expression analysis: Gene-specific mRNA quantitation was performed by real-time PCR with the Perkin Elmer Corp. chemistry on an ABI Prism 7700 Sequence detection system (Applied Biosystems, Foster City, Calif.) according to the manufacturer's instructions. Samples (50-100 ng) of total RNA were assayed in duplicate or triplicate in 50 ul reactions using one-step RT-PCR and the standard curve method to estimate specific mRNA concentrations. Sequences of gene-specific primer and probe sets were designed with Primer Express Software (Applied Biosystems, Foster City, Calif.). The human ABCA1 primer and probe sequences are: forward, CAACATGAATGCCATTTTCCAA, reverse, ATAATCCCCTGAACCCAAGGA, and probe, 6FAM-TAAAGCCATGCCCTCTGCAGGAACA-TAMRA. RT and PCR reactions were performed according to PE Applied Biosystem's protocol for Taqman Gold RT-PCR or Qiagen's protocol for Quantitect probe RT-PCR. Relative levels of ABCA1 mRNA are normalized using GAPDH mRNA or 18S rRNA probe/primer sets purchased commercially (Applied Biosystems, Foster City, Calif.).

### Statistics:

Mean, standard deviation and statistical significance of duplicate evaluations of RNA samples were assessed using ANOVA, one-way analysis of variance using SAS analysis.

### Reagents:

### —GAPDH Probe and Primers—Taqman GAPDH Control Reagents 402869 or 4310884E

### 18S Ribosomal RNA—Taqman 18S Control Reagents 4308329

### 10 Pack Taqman PCR Core Reagent Kit 402930

Qiagen Quantitect probe RT-PCR 204443.

### IV. Results

TABLE I

| | hLXRβ | hLXRα |
|---|---|---|
| | binding | binding |
| Example | IC50 (uM) | IC50 (uM) |
| 9 | 0.052 | 0.777 |
| 10 | 0.031 | 0.471 |
| 11 | 0.143 | 1.48 |
| 12 | 0.038 | 0.925 |
| 13 | 0.188 | 2.41 |
| 14 | 0.525 | 6.25 |
| 15 | 0.119 | 1.88 |
| 16 | 0.508 | 4.77 |
| 17 | 0.014 | 0.217 |
| 18 | 0.014 | 0.321 |
| 19 | 0.094 | 0.975 |
| 20 | 0.014 | 0.281 |
| 21 | 0.044 | 0.520 |
| 22 | 0.740 | 7.13 |
| 24 | 0.051 | 0.636 |
| 25 | 0.0034 | 0.136 |
| 26 | 0.005 | 0.221 |
| 27 | 0.008 | 0.144 |
| 28 | 0.00081 | 0.0059 |
| 29 | 0.0013 | 0.0100 |
| 30 | 0.0017 | 0.0158 |
| 38 | 0.135 | 2.33 |
| 39 | 3.65 | 10.24 |
| 42 | 0.409 | 0.920 |
| 43 | 0.0147 | 0.0399 |
| 44 | 0.0243 | 0.0714 |
| 45 | >1 | >1 |
| 46 | 0.0154 | 0.0649 |
| 47 | 0.151 | 2.14 |
| 48 | 0.0230 | 0.411 |
| 49 | 0.0075 | 0.2393 |
| 50 | 0.94 | 14.7 |
| 51 | 0.0796 | 1.92 |
| 52 | 0.089 | 2.63 |
| 53 | 0.137 | 1.86 |
| 54 | 3.06 | 41.1 |
| 55 | 1.10 | 28.5 |
| 56 | 4.19 | 36.5 |
| 57 | 8.43 | >1 |
| 58 | >1 | >1 |
| 59 | >1 | >1 |
| 60 | 0.194 | >1 |
| 61 | 0.0198 | 0.456 |
| 62 | 0.0915 | 2.81 |
| 63 | 0.0173 | 0.642 |
| 64 | 0.0151 | 0.399 |
| 65 | 0.0169 | 0.315 |
| 66 | 0.0088 | 0.292 |
| 67 | 0.0095 | 0.405 |
| 68 | 0.0071 | 0.406 |
| 69 | 0.161 | 2.70 |
| 70 | 0.107 | 4.67 |
| 71 | 0.0372 | 2.43 |
| 72 | 0.0435 | 1.5901 |
| 73 | 0.0365 | 1.88 |
| 74 | 0.0240 | 1.26 |
| 75 | 0.0065 | 0.222 |
| 76 | 0.0056 | 0.135 |
| 77 | 0.0061 | 0.160 |
| 78 | 0.0090 | 0.167 |
| 79 | 0.0237 | 0.745 |
| 80 | 0.0163 | 0.526 |
| 81 | 0.1523 | 2.39 |
| 82 | 0.0115 | 0.309 |
| 83 | 0.0068 | 0.218 |
| 84 | 0.0085 | 0.276 |
| 85 | 0.083 | 2.75 |
| 86 | 0.0091 | 0.102 |
| 87 | 8.33 | |
| 88 | >1 | >1 |
| 89 | >1 | >1 |
| 90 | 0.166 | 2.09 |
| 91 | >1 | >1 |
| 92 | 1.59 | >1 |
| 93 | 4.08 | >1 |
| 94 | >1 | >1 |
| 95 | 0.135 | 2.64 |
| 96 | NT | NT |
| 97 | NT | NT |
| 98 | 0.136 | >1 |
| 99 | >1 | >1 |
| 100 | 0.079 | >1 |
| 101 | >1 | >1 |
| 102 | 0.336 | >1 |
| 103 | 0.417 | >1 |
| 104 | >1 | NT |
| 105 | 0.23 | >1 |
| 106 | 0.43 | >1 |
| 107 | >1 | NT |
| 108 | 0.293 | >1 |
| 109 | 0.268 | >1 |
| 110 | >1 | NT |
| 111 | >1 | >1 |
| 112 | >1 | NT |
| 113 | NT | NT |
| 114 | 0.187 | >1 |
| 115 | >1 | >1 |
| 116 | 0.97 | >1 |
| 117 | 0.46 | >1 |
| 118 | >1 | >1 |
| 119 | 0.179 | >1 |
| 120 | 0.117 | >1 |
| 121 | 0.46 | >1 |
| 122 | 0.345 | >1 |
| 123 | >1 | NT |
| 124 | >1 | NT |
| 125 | 0.60 | NT |
| 126 | 0.014 | 0.29 |
| 127 | 0.113 | NT |
| 128 | 0.028 | 0.43 |
| 129 | 0.030 | 0.37 |
| 130 | 0.23 | >1 |
| 131 | 0.069 | >1 |
| 132 | >1 | NT |
| 133 | 0.0020 | 0.0093 |
| 134 | >1 | NT |
| 135 | NT | NT |
| 136 | >1 | NT |
| 137 | >1 | >1 |
| 138 | NT | NT |
| 139 | 0.279 | >1 |
| 140 | 0.069 | 0.91 |
| 141 | 0.70 | >1 |
| 142 | >1 | >1 |

TABLE II

Gene regulation by LXR
(Human)

| | EC50 | Agonism ABCA1 |
|---|---|---|
| Example | ABCA1 (uM) | (%) |
| 10 | 0.552 | 91 |
| 17 | 0.099 | 91 |
| 18 | 0.110 | 118 |
| 19 | 0.408 | 72 |
| 20 | 0.070 | 101 |
| 21 | 0.771 | 111 |
| 24 | 0.147 | 76 |
| 46 | 0.359 | 92 |
| 48 | 0.925 | 94 |
| 49 | 0.794 | 101 |
| 51 | 1.470 | 93 |
| 52 | 2.385 | 86 |
| 61 | 1.644 | 60 |
| 63 | 0.304 | 96 |
| 64 | 0.640 | 79 |
| 65 | 1.292 | 113 |
| 66 | 0.625 | 74 |
| 67 | 1.615 | 81 |
| 68 | 1.255 | 93 |
| 70 | 1.265 | 95 |
| 71 | 1.940 | 96 |
| 72 | 0.510 | 79 |
| 73 | 2.035 | 82 |
| 74 | 0.560 | 97 |
| 75 | 0.395 | 91 |
| 78 | 1.465 | 92 |
| 79 | 0.285 | 60 |
| 82 | 0.290 | 70 |
| 83 | 0.260 | 76 |
| 84 | 0.375 | 70 |
| 91 | 2.775 | 33 |
| 98 | 1.37 | 66 |
| 100 | 0.46 | 82 |
| 119 | 0.62 | 59 |
| 120 | 0.59 | 53 |
| 126 | 0.025 | 30 |
| 127 | 0.48 | 25 |
| 128 | 0.040 | 76 |
| 129 | 0.135 | 103 |
| 131 | 0.043 | 100 |
| 133 | 0.0100 | 98 |

Based on the results obtained in the standard pharmacological test procedures, the compounds of this invention can be useful in treating or inhibiting LXR mediated diseases. In particular, the compounds of this invention can be useful in the treatment and inhibition of atherosclerosis and atherosclerotic lesions, lowering LDL cholesterol levels, increasing HDL cholesterol levels, increasing reverse cholesterol transport, inhibiting cholesterol absorption, treatment or inhibition of cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A number of embodiments of the invention have been described. Nevertheless, it will be understood that various modifications may be made without departing from the spirit and scope of the invention. Accordingly, other embodiments are in the claims.

What is claimed is:

1. A compound having formula (I): 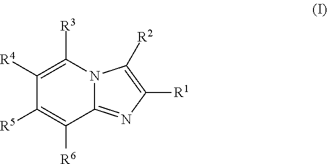
wherein:
R¹ is:
(i) hydrogen; or
(ii) C₁-C₆ alkyl or C₁-C₆ haloalkyl, each of which is optionally substituted with from 1-10 R^{a}; or
(iii) C₂-C₆ alkenyl or C₂-C₆ alkynyl, each of which is optionally substituted with from 1-10 R^{b}; or
(iv) C₃-C₁₀ cycloalkyl, C₃-C₁₀ cycloalkenyl, heterocyclyl including 3-10 atoms, heterocycloalkenyl including 3-10 atoms, C₇-C₁₁ aralkyl, or heteroaralkyl including 6-11 atoms, each of which is optionally substituted with from 1-10 R^{c}; or
(v) C₆-C₁₀ aryl or heteroaryl including 5-10 atoms, each of which is optionally substituted with from 1-10 R^{d}; or
(vi) halo;
R² is C₆-C₁₀ aryl or heteroaryl including 5-10 atoms, each of which is:
(i) substituted with 1 R⁷, and
(ii) optionally substituted with from 1-5 R^{e}; wherein:
R⁷ is WA, wherein:
W is a bond; —O—; —NR⁸—; C₁₋₆ alkylene, C₂₋₆ alkenylene, or C₂₋₆ alkynylene; —W¹(C₁₋₆ alkylene)-; or —(C₁₋₆ alkylene)W¹—;
W¹ is independently —O— or —NR⁸—;
R⁸ is hydrogen or C₁-C₆ alkyl;
A is C₆-C₁₀ aryl or heteroaryl including 5-10 atoms, each of which is:
(i) substituted with 1 R⁹, and
(ii) optionally further substituted with from 1-5 R^{g};
R⁹ is:
(i) —W²—S(O)ₙR¹⁰ or —W²—S(O)ₙNR¹¹R¹²; or
(ii) —W²—C(O)OR¹³; or
(iii) —W²—C(O)NR¹¹R¹²; or
(iv) C₁-C₁₂ alkyl or C₁-C₁₂ haloalkyl, each of which is:
(a) substituted with 1 R^{h}, and
(b) optionally further substituted with from 1-5 R^{a};
or
(v) —NR¹⁴R¹⁵;
wherein:
W² is a bond; C₁₋₆ alkylene; C₂₋₆ alkenylene; C₂₋₆ alkynylene; C₃₋₆ cycloalkylene; —O(C₁₋₆ alkylene)-; or —NR⁸(C₁₋₆ alkylene)-;
n is 1 or 2;
R¹⁰ is:
(i) C₁-C₆ alkyl or C₁-C₆ haloalkyl, each of which is optionally substituted with from 1-5 R^{a}; or
(ii) C₂-C₆ alkenyl or C₂-C₆ alkynyl, each of which is optionally substituted with from 1-5 R^{b}; or
(iii) C₃-C₁₀ cycloalkyl, C₃-C₁₀ cycloalkenyl, C₇-C₁₁ aralkyl, or heteroaralkyl including 6-11 atoms, each of which is optionally substituted with from 1-5 R^{c}; or
(iv) C₆-C₁₀ aryl or heteroaryl including 5-10 atoms, each of which is optionally substituted with from 1-5 R^{d};
R¹¹ and R¹² are each, independently:
(i) hydrogen;
(ii) C₁-C₆ alkyl or C₁-C₆ haloalkyl, each of which is optionally substituted with from 1-5 R^{a}; or
(iii) C₂-C₆ alkenyl or C₂-C₆ alkynyl, each of which is optionally substituted with from 1-5 R^{b}; or
(iv) C₃-C₁₀ cycloalkyl, C₃-C₁₀ cycloalkenyl, C₇-C₁₁ aralkyl, or heteroaralkyl including 6-11 atoms, each of which is optionally substituted with from 1-5 R^{c}; or
(v) C₆-C₁₀ aryl or heteroaryl including 5-10 atoms, each of which is optionally substituted with from 1-5 R^{d}; or
(vi) heterocyclyl including 3-10 atoms or a heterocycloalkenyl including 3-10 atoms, each of which is optionally substituted with from 1-5 R^{c};
or
R¹¹ and R¹² together with the nitrogen atom to which they are attached form a heterocyclyl including 3-10 atoms or a heterocycloalkenyl including 3-10 atoms, each of which is optionally substituted with from 1-5 R^{c};
R¹³ is:
(i) hydrogen;
(ii) C₁-C₆ alkyl or C₁-C₆ haloalkyl, each of which is optionally substituted with from 1-5 R^{a}; or
(iii) C₂-C₆ alkenyl or C₂-C₆ alkynyl, each of which is optionally substituted with from 1-5 R^{b}; or
(iv) C₃-C₁₀ cycloalkyl, C₃-C₁₀ cycloalkenyl, C₇-C₁₁ aralkyl, or heteroaralkyl including 6-11 atoms, each of which is optionally substituted with from 1-5 R^{c}; or
(v) C₆-C₁₀ aryl or heteroaryl including 5-10 atoms, each of which is optionally substituted with from 1-5 R^{d};
one of R¹⁴ and R¹⁵ is hydrogen or C₁-C₃ alkyl; and the other of R¹⁴ and R¹⁵ is:
(i) —S(O)ₙR¹⁰; or
(ii) —C(O)OR¹³; or
(iii) —C(O)NR¹¹R¹²; or
(iv) C₁-C₁₂ alkyl or C₁-C₁₂ haloalkyl, each of which is:
(a) substituted with 1 R^{h}, and
(b) optionally further substituted with from 1-5 R^{a};
each of R³, R⁴, and R⁵ is, independently:
(i) hydrogen; or
(ii) halo; or
(iii) C₁-C₆ alkyl or C₁-C₆ haloalkyl, each of which is optionally substituted with from 1-3 R^{a};
R⁶ is:
(i) hydrogen; or
(ii) halo; or
(iii) C₁-C₆ alkyl or C₁-C₆ haloalkyl, each of which is optionally substituted with from 1-3 R^{a}; or
(iv) nitro; C₁-C₆ alkoxy; C₁-C₆ haloalkoxy; C₁-C₆ thioalkoxy; C₁-C₆ thiohaloalkoxy; or cyano;
R^{a} at each occurrence is, independently:
(i) NR^{m}Rⁿ; hydroxy; C₁-C₆ alkoxy or C₁-C₆ haloalkoxy; C₆-C₁₀ aryloxy or heteroaryloxy including 5-10 atoms, each of which is optionally substituted with from 1-5 R^{d}; C₇-C₁₁ aralkoxy, heteroaralkoxy including 6-11 atoms, C₃-C₁₁ cycloalkoxy, C₃-C₁₁ cycloalkenyloxy, heterocyclyloxy including 3-10 atoms, or heterocycloalkenyloxy including 3-10 atoms, each of which is optionally substituted with from 1-5 R^{c}; or cyano; or
(ii) C₃-C₁₀ cycloalkyl, C₃-C₁₀ cycloalkenyl, heterocyclyl including 3-10 atoms, or heterocycloalkenyl including 3-10 atoms, each of which is optionally substituted with from 1-5 R^{c};
R^{b} at each occurrence is, independently:
(i) halo NR^{m}Rⁿ; hydroxy; C₁-C₆ alkoxy or C₁-C₆ haloalkoxy; C₆-C₁₀ aryloxy or heteroaryloxy including 5-10 atoms, each of which is optionally substituted with from 1-5 R^{d}; C₇-C₁₁ aralkoxy, heteroaralkoxy including 6-11 atoms, C₃-C₁₀ cycloalkoxy, C₃-C₁₀ cycloalkenyloxy, heterocyclyloxy including 3-10 atoms, or heterocycloalkenyloxy including 3-10 atoms, each of which is optionally substituted with from 1-5 R^{c};
(ii) C₃-C₁₀ cycloalkyl, C₃-C₁₀ cycloalkenyl, heterocyclyl including 3-10 atoms, or heterocycloalkenyl including 3-10 atoms, each of which is optionally substituted with from 1-5 R^{c}; or
(iii) C₆-C₁₀ aryl or heteroaryl including 5-10 atoms, each of which is optionally substituted with from 1-5 R^{d};
R^{c} at each occurrence is, independently:
(i) halo; NR^{m}Rⁿ; hydroxy; C₁-C₆ alkoxy or C₁-C₆ haloalkoxy; or
(ii) C₁-C₆ alkyl or C₁-C₆ haloalkyl, each of which is optionally substituted with from 1-5 R^{a}; or
(iii) C₂-C₆ alkenyl or C₂-C₆ alkynyl, each of which is optionally substituted with from 1-5 R^{b};
R^{d} at each occurrence is, independently:
(i) halo; NR^{m}Rⁿ; hydroxy; C₁-C₆ alkoxy or C₁-C₆ haloalkoxy; cyano; or
(ii) C₁-C₆ alkyl or C₁-C₆ haloalkyl, each of which is optionally substituted with from 1-5 R^{a}; or
(iii) C₂-C₆ alkenyl or C₂-C₆ alkynyl, each of which is optionally substituted with from 1-5 R^{b};
R^{e} at each occurrence is, independently, C₁-C₆ alkyl; C₁-C₆ haloalkyl; halo; hydroxyl; NR^{m}Rⁿ; C₁-C₆ haloalkoxy; or cyano;
R^{g} at each occurrence is, independently:
(i) halo; NR^{m}Rⁿ; hydroxy; C₁-C₆ alkoxy or C₁-C₆ haloalkoxy; cyano; or
(ii) C₁-C₆ alkyl or C₁-C₆ haloalkyl;
R^{h} at each occurrence is, independently, hydroxyl, C₁-C₆ alkoxy, or C₁-C₆ haloalkoxy; C₃-C₁₀ cycloalkoxy or C₃-C₁₀ cycloalkenyloxy, each of which is optionally substituted with from 1-5 R^{c}; or C₆-C₁₀ aryloxy or heteroaryloxy including 5-10 atoms, each of which is optionally substituted with from 1-5 R^{d};
each of R^{m} and Rⁿ at each occurrence is, independently, hydrogen, C₁-C₆ alkyl, or C₁-C₆ haloalkyl;
or an N-oxide and/or a pharmaceutically acceptable salt thereof.

2. The compound of claim 1, wherein R² is C₆-C₁₀ aryl, which is (a) substituted with 1 R⁷; and (b) optionally substituted with from 1-4 R^{e}.

3. The compound of claim 1, wherein R² is phenyl, which is (a) substituted with 1 R⁷; and (b) optionally substituted with 1 R^{e}.

4. The compound of claim 3, wherein R² has formula (A-2): 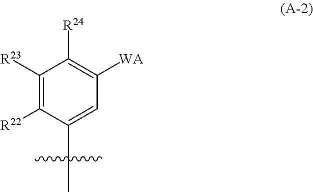
wherein:
(i) each of R²², R²³, and R²⁴ is hydrogen; or
(ii) one of R²², R²³, and R²⁴ is R^{e}, and the other two are hydrogen.

5. The compound of claim 4, wherein each of R²², R²³, and R²⁴ is hydrogen.

6. The compound of claim 1, wherein W is —O—.

7. The compound of claim 1, wherein A is C₆-C₁₀ aryl, which is (a) substituted with 1 R⁹; and (b) optionally substituted with from 1-4 R⁹.

8. The compound of claim 1, wherein A is phenyl, which is (a) substituted with 1 R⁹; and (b) optionally substituted with from 1-4 R^{g}.

9. The compound of claim 1, wherein A has formula (B-1): 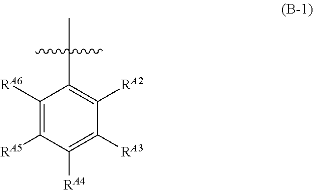
wherein:
one of R^{A3} and R^{A4} is R⁹, the other of R^{A3} and R^{A4} is hydrogen; and
each of R^{A2}, R^{A5}, and R^{A6} is, independently, hydrogen or R^{g}.

10. The compound of claim 1, wherein R⁹ is —W²—S(O)ₙR¹⁰.

11. The compound of claim 10, wherein W² is a bond, and n is 2.

12. The compound of claim 1, wherein R¹⁰ is C₁-C₆ alkyl, optionally substituted with from 1-2 R^{a}.

13. The compound of claim 1, wherein R⁹ is —W²—S(O)ₙNR¹¹R¹².

14. The compound of claim 13, wherein W² is a bond, and n is 2.

15. The compound of claim 1, wherein each of R¹¹ and R¹² is, independently:
(i) hydrogen; or
(ii) C₁-C₆ alkyl; or
(iii) C₇-C₁₁ aralkyl, optionally substituted with from 1-5 R^{c}.

16. The compound of claim 1, wherein R² has formula (C-1): 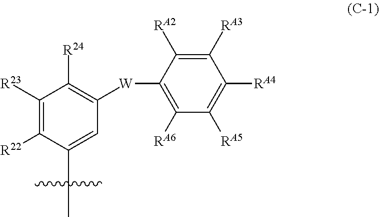
wherein:
(i) each of R²², R²³, and R²⁴ is hydrogen; or
(ii) one of R²², R²³, and R²⁴ is R^{e}, and the other two are hydrogen;
and
one of R^{A2}, R^{A3}, R^{A4}, R^{A5}, and R^{A6} is R⁹, and the others are each, independently, hydrogen or R^{g}.

17. The compound of claim 16, wherein each of R²², R²³, and R²⁴ is hydrogen.

18. The compound of claim 16, wherein one of R²², R²³, and R²⁴ is R^{e}, and the other two are hydrogen.

19. The compound of claim 18, wherein R²² is R^{e}, and each of R²³ and R²⁴ is hydrogen.

20. The compound of claim 19, wherein R²² is halo.

21. The compound of claim 1, wherein W is —O—.

22. The compound of claim 1, wherein W is a bond, C₁₋₆ alkylene, or —W¹(C₁₋₆ alkylene)-.

23. The compound of claim 1, wherein R⁹ is —W²—S(O)ₙR¹⁰ or —W²—S(O)ₙNR¹¹R¹².

24. The compound of claim 1, wherein one of R^{A3} and R^{A4} is R⁹, and the other of R^{A3} and R^{A4} is hydrogen; and each of R^{A2}, R^{A5}, and R^{A6} is, independently, hydrogen or R^{g}.

25. The compound of claim 24, wherein R^{A3} is R⁹, and R^{A4} is hydrogen.

26. The compound of claim 25, wherein R⁹ is —W²—S(O)ₙR¹⁰.

27. The compound of claim 26, wherein W² is a bond; n is 2; and R¹⁰ is C₁-C₆ alkyl, optionally substituted with from 1-2 R^{a}.

28. The compound of claim 27, wherein R¹⁰ is C₁-C₅ alkyl.

29. The compound of claim 28, wherein R¹⁰ is CH₃.

30. The compound of claim 28, wherein R¹ is CH₃CH₂.

31. The compound of claim 28, wherein R¹ is CH(CH₃)₂.

32. The compound of claim 27, wherein R¹⁰ is C₂-C₆ alkyl substituted with 1 R^{a}.

33. The compound of claim 32, wherein R^{a} is hydroxyl, C₁-C₃ alkoxy, or NR^{m}Rⁿ.

34. The compound of claim 25, wherein R⁹ is —W²—S(O)ₙNR¹¹R¹².

35. The compound of claim 34, wherein W² is a bond; n is 2, and each of R¹¹ and R¹² is, independently:
(i) hydrogen; or
(ii) C₁-C₆ alkyl; or
(iii) C₇-C₁₁ aralkyl, optionally substituted with from 1-5 R^{c}.

36. The compound of claim 1, wherein each of R^{A2}, R^{A5}, and R^{A6} is hydrogen.

37. The compound of claim 1, wherein R^{A5} is R^{g}, and each of R^{A2} and R^{A6} is hydrogen.

38. The compound of claim 1, wherein R¹ is C₁-C₆ alkyl or C₁-C₆ haloalkyl, each of which is optionally substituted with from 1-10 R^{a}.

39. The compound of claim 38, wherein R¹ is C₁-C₆ alkyl.

40. The compound of claim 39, wherein R¹ is CH₃CH₂ or CH(CH₃)₂.

41. The compound of claim 38, wherein R¹ is C₁-C₆ alkyl substituted with 1 R^{a}.

42. The compound of claim 38, wherein R^{a} is heterocyclyl including 5 or 6 ring atoms, which is which is optionally substituted with from 1-5 R^{c}.

43. The compound of claim 1, wherein R¹ is C₆-C₁₀ aryl or heteroaryl including 5-10 atoms, each of which is optionally substituted with from 1-5 R^{d}.

44. The compound of claim 43, wherein R¹ is phenyl, which is optionally substituted with from 1-5 R^{d}.

45. The compound of claim 1, wherein R¹ is C₇-C₁₁ aralkyl or heteroaralkyl including 6-11 atoms, each of which is optionally substituted with from 1-5 R^{c}.

46. The compound of claim 45, wherein R¹ is benzyl, which is optionally substituted with from 1-5 R^{c}.

47. The compound of claim 1, wherein each of R³, R⁴, and R⁵ is hydrogen.

48. The compound of claim 1, wherein R⁶ is C₁-C₃ perfluoroalkyl.

49. The compound of claim 48, wherein R⁶ is CF₃.

50. The compound of claim 1, wherein R⁶ is cyano.

51. The compound of claim 1, wherein R⁶ is halo.

52. The compound of claim 1, wherein the compound has formula (VI): 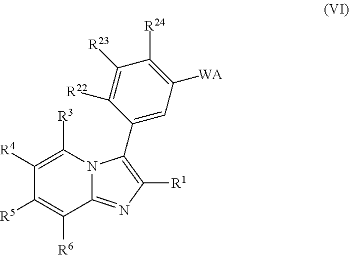
wherein:
R¹ is:
(i) C₁-C₆ alkyl or C₁-C₆ haloalkyl, each of which is optionally substituted with from 1-10 R^{a}; or
(ii) phenyl or heteroaryl including 5-6 atoms, each of which is optionally substituted with from 1-5 R^{d}; or
(iii) C₇-C₁₁ aralkyl or heteroaralkyl including 6-11 atoms, each of which is optionally substituted with from 1-3 R^{c};
each of R³, R⁴, and R⁵ is hydrogen;
R⁶ is:
(ii) halo; or
(iii) C₁-C₃ alkyl or C₁-C₃ haloalkyl, each of which is optionally substituted with from 1-3 R^{a}; or
(iv) cyano;
each of R²², R²³, and R²⁴ is hydrogen; or
one of R²², R²³, and R²⁴ is R^{e}, and the other two are hydrogen;
W is a bond, —O—, —OCH₂—, or —CH₂—;
A has formula (B-1), wherein one of R^{A3} and R^{A4} is R⁹, and the other of R^{A3} and R^{A4} is hydrogen; and each of R^{A2}, R^{A5}, and R^{A6} is, independently, hydrogen or R^{g} 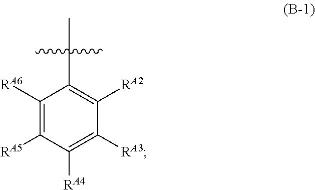
and
R⁹ is —W²—S(O)ₙR¹² or —W²—S(O)ₙNR¹¹R¹².

53. The compound of claim 1, wherein the compound is selected from:
2-methyl-3-(3-(3-(methylsulfonyl)phenoxy)phenyl)-8-(trifluoromethyl)imidazo[1,2-a]pyridine;
2-methyl-3-(3-(3-(ethylsulfonyl)phenoxy)phenyl)-8-(trifluoromethyl)imidazo[1,2-a]pyridine;
2-methyl-3-(3-(3-(propylsulfonyl)phenoxy)phenyl)-8-(trifluoromethyl)imidazo[1,2-a]pyridine;
2-methyl-3-(3-(3-(isopropylsulfonyl)phenoxy)phenyl)-8-(trifluoromethyl)imidazo[1,2-a]pyridine;
3-[(3-{3-[2-methyl-8-(trifluoromethyl)imidazo[1,2-a]pyridin-3-yl]phenoxy}phenyl)sulfonyl]propan-1-ol;
2-methyl-4-[(3-{3-[2-methyl-8-(trifluoromethyl)imidazo[1,2-a]pyridin-3-yl]phenoxy}phenyl)sulfonyl]butan-2-ol;
3-{3-[3-fluoro-5-(methylsulfonyl)phenoxy]phenyl}-2-methyl-8-(trifluoromethyl)imidazo[1,2-a]pyridine;
3-{3-[4-(sec-butylsulfonyl)phenoxy]phenyl}-2-methyl-8-(trifluoromethyl)imidazo[1,2-a]pyridine;
2-ethyl-3-(3-(3-(methylsulfonyl)phenoxy)phenyl)-8-(trifluoromethyl)imidazo[1,2-a]pyridine;
2-ethyl-3-(3-(3-(ethylsulfonyl)phenoxy)phenyl)-8-(trifluoromethyl)imidazo[1,2-a]pyridine;
2-ethyl-3-(3-(3-(propylsulfonyl)phenoxy)phenyl)-8-(trifluoromethyl)imidazo[1,2-a]pyridine;
2-ethyl-3-(3-(3-(isopropylsulfonyl)phenoxy)phenyl)-8-(trifluoromethyl)imidazo[1,2-a]pyridine;
3-(3-(3-(2-ethyl-8-(trifluoromethyl)imidazo[1,2-a]pyridin-3-yl)phenoxy)phenylsulfonyl)propan-1-ol;
4-(3-(3-(2-ethyl-8-(trifluoromethyl)imidazo[1,2-a]pyridin-3-yl)phenoxy)phenylsulfonyl)-2-methylbutan-2-ol;
5-(3-(3-(2-ethyl-8-(trifluoromethyl)imidazo[1,2-a]pyridin-3-yl)phenoxy)phenylsulfonyl)pentan-1-ol;
2-ethyl-3-(3-(3-fluoro-5-(methylsulfonyl)phenoxy)phenyl)-8-(trifluoromethyl)imidazo[1,2-a]pyridine;
2-isopropyl-3-(3-(3-(methylsulfonyl)phenoxy)phenyl)-8-(trifluoromethyl)imidazo[1,2-a]pyridine;
2-isopropyl-3-(3-(3-(ethylsulfonyl)phenoxy)phenyl)-8-(trifluoromethyl)imidazo[1,2-a]pyridine;
2-isopropyl-3-(3-(3-(isopropylsulfonyl)phenoxy)phenyl)-8-(trifluoromethyl)imidazo[1,2-a]pyridine;
2-benzyl-3-(3-(3-(methylsulfonyl)phenoxy)phenyl)-8-(trifluoromethyl)imidazo[1,2-a]pyridine;
2-benzyl-3-(3-(3-(ethyl sulfonyl)phenoxy)phenyl)-8-(trifluoromethyl)imidazo[1,2-a]pyridine;
2-benzyl-3-(3-(3-(isopropylsulfonyl)phenoxy)phenyl)-8-(trifluoromethyl)imidazo[1,2-a]pyridine;
3-(3-(8-chloro-2-isopropylimidazo[1,2-a]pyridin-3-yl)phenoxy)-N,N-bis(4-methoxybenzyl)benzenesulfonamide;
4-(3-(8-chloro-2-isopropylimidazo[1,2-a]pyridin-3-yl)phenoxy)-N,N-bis(4-methoxybenzyl)benzenesulfonamide;
3-(3-(8-chloro-2-isopropylimidazo[1,2-a]pyridin-3-yl)phenoxy)benzenesulfonamide;
4-(3-(8-chloro-2-isopropylimidazo[1,2-a]pyridin-3-yl)phenoxy)benzenesulfonamide;
3-{3-[2-ethyl-8-(trifluoromethyl)imidazo[1,2-a]pyridin-3-yl]phenoxy}-N-(4-methoxybenzyl)-N-methylbenzenesulfonamide;
3-{3-[2-isopropyl-8-(trifluoromethyl)imidazo[1,2-a]pyridin-3-yl]phenoxy}-N,N-bis(4-methoxybenzyl)benzenesulfonamide;
3-[3-(8-chloro-2-ethylimidazo[1,2-a]pyridin-3-yl)phenoxy]-N,N-bis(4-methoxybenzyl)benzenesulfonamide;
3-[3-(8-chloro-2-ethylimidazo[1,2-a]pyridin-3-yl)phenoxy]-N-(4-methoxybenzyl)-N-methylbenzenesulfonamide;
3-[3-(8-chloro-2-isopropylimidazo[1,2-a]pyridin-3-yl)phenoxy]-N-(4-methoxybenzyl)-N-methylbenzenesulfonamide;
3-[3-(8-cyano-2-ethylimidazo[1,2-a]pyridin-3-yl)phenoxy]-N,N-bis(4-methoxybenzyl)benzenesulfonamide;
3-[3-(8-cyano-2-ethylimidazo[1,2-a]pyridin-3-yl)phenoxy]-N-(4-methoxybenzyl)-N-methylbenzenesulfonamide;
3-[3-(8-chloro-2-ethylimidazo[1,2-a]pyridin-3-yl)phenoxy]benzenesulfonamide;
3-[3-(8-chloro-2-ethylimidazo[1,2-a]pyridin-3-yl)phenoxy]-N-methylbenzenesulfonamide;
3-[3-(8-chloro-2-isopropylimidazo[1,2-a]pyridin-3-yl)phenoxy]-N-methylbenzenesulfonamide;
3-[3-(8-cyano-2-ethylimidazo[1,2-a]pyridin-3-yl)phenoxy]benzenesulfonamide;
3-[3-(8-cyano-2-ethylimidazo[1,2-a]pyridin-3-yl)phenoxy]-N-methylbenzenesulfonamide;
2-isopropyl-3-(3-{[3-(methylsulfonyl)benzyl]oxy}phenyl)-8-(trifluoromethyl)imidazo[1,2-a]pyridine;
8-chloro-2-isopropyl-3-(3-{[3-(methylsulfonyl)benzyl]oxy}phenyl)imidazo[1,2-a]pyridine;
2-ethyl-3-(3-{[3-(methylsulfonyl)benzyl]oxy}phenyl)imidazo[1,2-a]pyridine-8-carbonitrile;
2-isopropyl-3-(3-{[3-(methylsulfonyl)benzyl]oxy}phenyl)imidazo[1,2-a]pyridine-8-carbonitrile;
2-isopropyl-3-(3-{[4-(methylsulfonyl)benzyl]oxy}phenyl)-8-(trifluoromethyl)imidazo[1,2-a]pyridine;
8-chloro-2-isopropyl-3-(3-{[4-(methylsulfonyl)benzyl]oxy}phenyl)imidazo[1,2-a]pyridine;
2-ethyl-3-(3-{[4-(methylsulfonyl)benzyl]oxy}phenyl)imidazo[1,2-a]pyridine-8-carbonitrile;
2-isopropyl-3-(3-{[4-(methylsulfonyl)benzyl]oxy}phenyl)imidazo[1,2-a]pyridine-8-carbonitrile;
2-isopropyl-8-(trifluoromethyl)-3-(3-{3-[(trifluoromethyl)sulfonyl]phenoxy}phenyl)imidazo[1,2-a]pyridine;
3-[(3-{3-[2-isopropyl-8-(trifluoromethyl)imidazo[1,2-a]pyridin-3-yl]phenoxy}phenyl)sulfonyl]propan-1-ol;
3-[(3-{3-[2-isopropyl-8-(trifluoromethyl)imidazo[1,2-a]pyridin-3-yl]phenoxy}phenyl)sulfonyl]-N-methylpropan-1-amine;
8-chloro-2-ethyl-3-{3-[3-(methylsulfonyl)phenoxy]phenyl}imidazo[1,2-a]pyridine;
8-chloro-2-ethyl-3-{3-[3-(ethylsulfonyl)phenoxy]phenyl}imidazo[1,2-a]pyridine;
8-chloro-2-ethyl-3-{3-[3-(isopropylsulfonyl)phenoxy]phenyl}imidazo[1,2-a]pyridine;
8-chloro-2-isopropyl-3-{3-[3-(methylsulfonyl)phenoxy]phenyl}imidazo[1,2-a]pyridine;
8-chloro-3-{3-[3-(ethylsulfonyl)phenoxy]phenyl}-2-isopropylimidazo[1,2-a]pyridine;
8-chloro-2-isopropyl-3-{3-[3-(isopropylsulfonyl)phenoxy]phenyl}imidazo[1,2-a]pyridine;
2-ethyl-3-{3-[3-(methylsulfonyl)phenoxy]phenyl}imidazo[1,2-a]pyridine-8-carbonitrile;
2-ethyl-3-{3-[3-(ethylsulfonyl)phenoxy]phenyl}imidazo[1,2-a]pyridine-8-carbonitrile;
2-ethyl-3-{3-[3-(isopropylsulfonyl)phenoxy]phenyl}imidazo[1,2-a]pyridine-8-carbonitrile;
2-isopropyl-3-{3-[3-(methylsulfonyl)phenoxy]phenyl}imidazo[1,2-a]pyridine-8-carbonitrile;
3-{3-[3-(ethylsulfonyl)phenoxy]phenyl}-2-isopropylimidazo[1,2-a]pyridine-8-carbonitrile;
2-isopropyl-3-{3-[3-(isopropylsulfonyl)phenoxy]phenyl}imidazo[1,2-a]pyridine-8-carbonitrile;
2-tert-butyl-3-{3-[3-(methylsulfonyl)phenoxy]phenyl}-8-(trifluoromethyl)imidazo[1,2-a]pyridine;
2-tert-butyl-3-{3-[3-(ethylsulfonyl)phenoxy]phenyl}-8-(trifluoromethyl)imidazo[1,2-a]pyridine;
2-tert-butyl-3-{3-[3-(isopropylsulfonyl)phenoxy]phenyl}-8-(trifluoromethyl)imidazo[1,2-a]pyridine;
3-[(3-{3-[2-tert-butyl-8-(trifluoromethyl)imidazo[1,2-a]pyridin-3-yl]phenoxy}phenyl)sulfonyl]propan-1-ol;
2-tert-butyl-3-{3-[3-(ethylsulfonyl)phenoxy]phenyl}imidazo[1,2-a]pyridine-8-carbonitrile;
2-tert-butyl-3-{3-[3-(isopropylsulfonyl)phenoxy]phenyl}imidazo[1,2-a]pyridine-8-carbonitrile;
2-tert-butyl-3-(3-{3-[(3-hydroxypropyl)sulfonyl]phenoxy}phenyl)imidazo[1,2-a]pyridine-8-carbonitrile;
3-{2-chloro-5-[3-(methylsulfonyl)phenoxy]phenyl}-2-isopropylimidazo[1,2-a]pyridine-8-carbonitrile;
3-{2-chloro-5-[3-(ethylsulfonyl)phenoxy]phenyl}-2-isopropylimidazo[1,2-a]pyridine-8-carbonitrile;
3-{2-chloro-5-[3-(isopropylsulfonyl)phenoxy]phenyl}-2-isopropylimidazo[1,2-a]pyridine-8-carbonitrile;
2-chloro-3-{3-[3-(methylsulfonyl)phenoxy]phenyl}-8-(trifluoromethyl)imidazo[1,2-a]pyridine;
3-{3-[3-(methylsulfonyl)phenoxy]phenyl}-2-phenyl-8-(trifluoromethyl)imidazo[1,2-a]pyridine;
2-ethyl-3-{6-[3-(methylsulfonyl)phenyl]pyridin-3-yl}-8-(trifluoromethyl)imidazo[1,2-a]pyridine;
2-ethyl-3-{6-[3-(methylsulfonyl)phenoxy]pyridin-2-yl}-8-(trifluoromethyl)imidazo[1,2-a]pyridine;
2-isopropyl-3-{6-[3-(methylsulfonyl)phenoxy]pyridin-2-yl}-8-(trifluoromethyl)imidazo[1,2-a]pyridine;
2-isopropyl-3-(3′-(methylsulfonyl)biphenyl-4-yl)-8-(trifluoromethyl)imidazo[1,2-a]pyridine;
2-isopropyl-3-(4′-(isopropylsulfonyl)biphenyl-4-yl)-8-(trifluoromethyl)imidazo[1,2-a]pyridine;
2-ethyl-3-(4′-(isopropylsulfonyl)biphenyl-4-yl)-8-(trifluoromethyl)imidazo[1,2-a]pyridine;
2-ethyl-3-(4′-(methylsulfonyl)biphenyl-4-yl)-8-(trifluoromethyl)imidazo[1,2-a]pyridine;
2-isopropyl-3-(4′-(methylsulfonyl)biphenyl-4-yl)-8-(trifluoromethyl)imidazo[1,2-a]pyridine;
2-ethyl-3-(3′-(methylsulfonyl)biphenyl-4-yl)-8-(trifluoromethyl)imidazo[1,2-a]pyridine;
4-[3-(8-chloro-2-isopropylimidazo[1,2-a]pyridin-3-yl)phenoxy]-N,N-bis(4-methoxybenzyl)benzenesulfonamide;
3-[3-(8-chloro-2-isopropylimidazo[1,2-a]pyridin-3-yl)phenoxy]-N,N-bis(4-methoxybenzyl)benzenesulfonamide;
3-[3-(8-chloro-2-isopropylimidazo[1,2-a]pyridin-3-yl)phenoxy]benzenesulfonamide;
4-[3-(8-chloro-2-isopropylimidazo[1,2-a]pyridin-3-yl)phenoxy]benzenesulfonamide;
2-ethyl-3-{3-[3-(methylsulfonyl)benzyl]phenyl}-8-(trifluoromethyl)imidazo[1,2-a]pyridine;
2-ethyl-3-{4-[3-(methylsulfonyl)benzyl]phenyl}-8-(trifluoromethyl)imidazo[1,2-a]pyridine;
2-ethyl-3-{4-[3-(methylsulfonyl)phenoxy]phenyl}-8-(trifluoromethyl)imidazo[1,2-a]pyridine;
8-chloro-2-ethyl-3-{4-[3-(methylsulfonyl)phenoxy]phenyl}imidazo[1,2-a]pyridine;
2-ethyl-3-{4-[3-(methylsulfonyl)phenoxy]phenyl}imidazo[1,2-a]pyridine-8-carbonitrile;
2-ethyl-3-{4-[3-(ethylsulfonyl)phenoxy]phenyl}-8-(trifluoromethyl)imidazo[1,2-a]pyridine;
8-chloro-2-ethyl-3-{4-[3-(ethylsulfonyl)phenoxy]phenyl}imidazo[1,2-a]pyridine;
2-ethyl-3-{4-[3-(ethylsulfonyl)phenoxy]phenyl}imidazo[1,2-a]pyridine-8-carbonitrile;
2-ethyl-3-(3-{[3-(methylsulfonyl)phenoxy]methyl}phenyl)-8-(trifluoromethyl)imidazo[1,2-a]pyridine;
8-chloro-2-ethyl-3-(3-{[3-(methylsulfonyl)phenoxy]methyl}phenyl)imidazo[1,2-a]pyridine;
2-ethyl-3-(3-{[3-(methylsulfonyl)phenoxy]methyl}phenyl)imidazo[1,2-a]pyridine-8-carbonitrile;
2-ethyl-3-(4-{[3-(methylsulfonyl)phenoxy]methyl}phenyl)-8-(trifluoromethyl)imidazo[1,2-a]pyridine;
8-chloro-2-ethyl-3-(4-{[3-(methylsulfonyl)phenoxy]methyl}phenyl)imidazo[1,2-a]pyridine;
2-ethyl-3-(4-{[3-(methylsulfonyl)phenoxy]methyl}phenyl)imidazo[1,2-a]pyridine-8-carbonitrile;
2-isopropyl-3-(3-{3-[methylsulfonyl)methyl]phenoxy}phenyl)-8-(trifluoromethyl)imidazo[1,2-a]pyridine;
2-isopropyl-3-(3-{3-[methylsulfonyl)methyl]phenoxy}phenyl)imidazo[1,2-a]pyridine-8-carbonitrile;
2-tert-butyl-3-(3-{3-[methylsulfonyl)methyl]phenoxy}phenyl)imidazo[1,2-a]pyridine-8-carbonitrile;
8-chloro-2-isopropyl-3-(3-{3-[methylsulfonyl)methyl]phenoxy}phenyl)imidazo[1,2-a]pyridine;
2-ethyl-3-(3-{3-[methylsulfonyl)methyl]phenoxy}phenyl)imidazo[1,2-a]pyridine-8-carbonitrile;
2-(1-methylethyl)-3-{4-[3-(methylsulfonyl)phenoxy]phenyl}-8-(trifluoromethyl)imidazo[1,2-a]pyridine;
3-{4-[3-(ethylsulfonyl)phenoxy]phenyl}-2-(1-methylethyl)-8-(trifluoromethyl)imidazo[1,2-a]pyridine;
8-chloro-2-(1-methylethyl)-3-{4-[3-(methylsulfonyl)phenoxy]phenyl}imidazo[1,2-a]pyridine;
8-chloro-3-{4-[3-(ethylsulfonyl)phenoxy]phenyl}-2-(1-methylethyl)imidazo[1,2-a]pyridine;
2-(1-methylethyl)-3-{4-[3-(methylsulfonyl)phenoxy]phenyl}imidazo[1,2-a]pyridine-8-carbonitrile;
3-{4-[3-(ethylsulfonyl)phenoxy]phenyl}-2-(1-methylethyl)imidazo[1,2-a]pyridine-8-carbonitrile;
3-{4-[3-(methylsulfonyl)phenoxy]phenyl}-2,8-bis(trifluoromethyl)imidazo[1,2-a]pyridine;
2-(2-methoxyphenyl)-3-{4-[3-(methylsulfonyl)phenoxy]phenyl}-8-(trifluoromethyl)imidazo[1,2-a]pyridine;
3-{4-[3-(methylsulfonyl)phenoxy]phenyl}-2-thiophen-3-yl-8-(trifluoromethyl)imidazo[1,2-a]pyridine;
2-(butoxymethyl)-3-{4-[3-(methylsulfonyl)phenoxy]phenyl}-8-(trifluoromethyl)imidazo[1,2-a]pyridine;
2-(1H-imidazol-1-ylmethyl)-3-{3-[3-(methylsulfonyl)phenoxy]phenyl}-8-(trifluoromethyl)imidazo[1,2-a]pyridine;
2-[(2-methyl-1H-imidazol-1-yl)methyl]-3-{3-[3-(methylsulfonyl)phenoxy]phenyl}-8-(trifluoromethyl)imidazo[1,2-a]pyridine;
3-{3-[3-(methylsulfonyl)phenoxy]phenyl}-2-(piperidin-1-ylmethyl)-8-(trifluoromethyl)imidazo[1,2-a]pyridine;
2-[(4-methylpiperazin-1-yl)methyl]-3-{3-[3-(methylsulfonyl)phenoxy]phenyl}-8-(trifluoromethyl)imidazo[1,2-a]pyridine;
3-{3-[3-(methylsulfonyl)phenoxy]phenyl}-2-(1,3-thiazolidin-3-ylmethyl)-8-(trifluoromethyl)imidazo[1,2-a]pyridine;
N-methyl-1-[3-{3-[3-(methylsulfonyl)phenoxy]phenyl}-8-(trifluoromethyl)imidazo[1,2-a]pyridin-2-yl]methanamine;
N-{[3-{3-[3-(methylsulfonyl)phenoxy]phenyl}-8-(trifluoromethyl)imidazo[1,2-a]pyridin-2-yl]methyl}ethanamine;
N-{[3-{3-[3-(methylsulfonyl)phenoxy]phenyl}-8-(trifluoromethyl)imidazo[1,2-a]pyridin-2-yl]methyl}propan-2-amine;
N,N-dimethyl-1-[3-{3-[3-(methylsulfonyl)phenoxy]phenyl}-8-(trifluoromethyl)imidazo[1,2-a]pyridin-2-yl]methanamine;
N-ethyl-N-{[3-{3-[3-(methylsulfonyl)phenoxy]phenyl}-8-(trifluoromethyl)imidazo[1,2-a]pyridin-2-yl]methyl}ethanamine;
3-{3-[3-(methylsulfonyl)phenoxy]phenyl}-2-(pyrrolidin-1-ylmethyl)-8-(trifluoromethyl)imidazo[1,2-a]pyridine;
4-[(3-{3-[2-ethyl-8-(trifluoromethyl)imidazo[1,2-a]pyridin-3-yl]phenoxy}phenyl)sulfonyl]butanenitrile;
4-{3-[2-ethyl-8-(trifluoromethyl)imidazo[1,2-a]pyridin-3-yl]phenoxy}-2-(methylsulfonyl)benzonitrile; and
2-(methylsulfonyl)-4-{3-[2-methyl-8-(trifluoromethyl)imidazo[1,2-a]pyridin-3-yl]phenoxy}benzonitrile;
or an N-oxide and/or a pharmaceutically acceptable salt thereof.

54. A composition comprising a compound of claim 1 or a pharmaceutically acceptable salt thereof and a pharmaceutically acceptable carrier.

55. A method of preventing or treating a Liver X receptor-mediated disease or disorder, the method comprising administering to a subject in need of such treatment an effective amount of a compound of claim 1 or a pharmaceutically acceptable salt thereof.

56. A method of preventing or treating atherosclerosis, the method comprising administering to a subject in need of such treatment an effective amount of a compound of claim 1 or a pharmaceutically acceptable salt thereof.

57. A method of preventing or treating a cardiovascular disease, the method comprising administering to a subject in need of such treatment an effective amount of a compound of claim 1 or a pharmaceutically acceptable salt thereof

58. The method of claim 57, wherein the cardiovascular disease is acute coronary syndrome or restenosis.

59. The method of claim 57, wherein the cardiovascular disease is coronary artery disease.

60. A method of preventing or treating Syndrome X, the method comprising administering to a subject in need of such treatment an effective amount of a compound of claim 1 or a pharmaceutically acceptable salt thereof.

61. A method of preventing or treating obesity, the method comprising administering to a subject in need of such treatment an effective amount of a compound of claim 1 or a pharmaceutically acceptable salt thereof.

62. A method of preventing or treating one or more lipid disorders selected from dyslipidemia, hyperlipidemia, hypertriglyceridemia, hypercholesterolemia, low HDL and/or high LDL, the method comprising administering to a subject in need of such treatment an effective amount of a compound of claim 1 or a pharmaceutically acceptable salt thereof.

63. A method of preventing or treating Alzheimer's disease, the method comprising administering to a subject in need of such treatment an effective amount of a compound of claim 1 or a pharmaceutically acceptable salt thereof.

64. A method of preventing or treating type I or type II diabetes, the method comprising administering to a subject in need of such treatment an effective amount of a compound of claim 1 or a pharmaceutically acceptable salt thereof.

65. A method of preventing or treating an inflammatory disease, the method comprising administering to a subject in need of such treatment an effective amount of a compound of claim 1 or a pharmaceutically acceptable salt thereof.

66. The method of claim 65, wherein the inflammatory disease is rheumatoid arthritis.

67. A method of treating a connective tissue disease, the method comprising administering to a mammal in need thereof an effective amount of a compound of claim 1 or a pharmaceutically acceptable salt thereof.

68. The method of claim 67, wherein the compound of formula (I) inhibits cartilage degradation and induces cartilage regeneration.

69. The method of claim 68, wherein the compound of formula (I) inhibits aggrecanase activity.

70. The method of claim 68, wherein the compound of formula (I) inhibits elaboration of pro-inflammatory cytokines in osteoarthritic lesions.

71. The method of claim 67, wherein the connective tissue disease is osteoarthritis or tendonitis.

72. The method of claim 67, wherein the mammal is a human.

73. A method of treating skin aging, the method comprising administering to a mammal in need thereof an effective amount of a compound of claim 1 or a pharmaceutically acceptable salt thereof.

74. The method of claim 73, wherein the mammal is a human.

75. The method of claim 73, wherein the compound of formula (I) is topically administered.

76. The method of claim 73, wherein the skin aging is derived from chronological aging, photoaging, steroid-induced skin thinning, or a combination thereof.
